(19) United States
(12) Patent Application Publication
(10) Pub. No.: US 2025/0320298 A1
(43) Pub. Date: Oct. 16, 2025

(54) DOSING FOR TREATMENT WITH ANTI-FCRH5/ANTI-CD3 BISPECIFIC ANTIBODIES

(71) Applicants: Genentech, Inc., South San Francisco, CA (US); Hoffmann-La Roche Inc., Little Falls, NJ (US)

(72) Inventors: Teemu Tapani JUNTTILA, San Mateo, CA (US); Divya Anthony SAMINENI, San Francisco, CA (US); Monica Etelina SUSILO, El Granada, CA (US); Elisabeth WASSNER, Mulhouse (FR); James Niall COOPER, Missoula, MT (US)

(21) Appl. No.: 19/016,060

(22) Filed: Jan. 10, 2025

Related U.S. Application Data
(63) Continuation of application No. PCT/US2023/070113, filed on Jul. 13, 2023.
(60) Provisional application No. 63/368,352, filed on Jul. 13, 2022.

(51) Int. Cl. C07K 16/28

(52) U.S. Cl. CPC C07K 16/283; C07K 16/2809; C07K 2317/31

(57) ABSTRACT

The invention provides methods of dosing for the treatment of cancers, such as multiple myelomas, with anti-fragment crystallizable receptor-like 5 (FcRH5)/anti-cluster of differentiation 3 (CD3) bispecific antibodies.

### SEQUENCE LISTING

The instant application contains a Sequence Listing which has been submitted electronically in XML format and is hereby incorporated by reference in its entirety. Said XML copy, created on Jan. 8, 2025, is named 50474-297003_Sequence_Listing_1_8_25.xml and is 41,594 bytes in size.

### FIELD OF THE INVENTION

The present invention relates to the treatment of cancers, such as B cell proliferative disorders. More specifically, the invention concerns the treatment of human patients having multiple myeloma (MM) using anti-fragment crystallizable receptor-like 5 (FcRH5)/anti-cluster of differentiation 3 (CD3) bispecific antibodies.

### BACKGROUND

Cancer remains one of the most deadly threats to human health. In the U.S., cancer affects more than 1.7 million new patients each year and is the second leading cause of death after heart disease, accounting for approximately one in four deaths.

Hematologic cancers, in particular, are the second leading cause of cancer-related deaths. Hematologic cancers include multiple myeloma (MM), a neoplasm characterized by the proliferation and accumulation of malignant plasma cells. Worldwide, approximately 160,000 people are diagnosed with MM annually. MM remains incurable despite advances in treatment, with an estimated median survival of 8-10 years for standard-risk myeloma and 2-3 years for high-risk disease, despite receipt of an autologous stem cell transplant. Despite the significant improvement in patient survival over the past 20 years, only 10-15% of patients achieve or exceed expected survival compared with the matched general population. Increased survival has been achieved with the introduction of proteasome inhibitors (Pis), immunomodulatory drugs (IMiDs), and monoclonal antibodies (mAbs). Nevertheless, most patients (if not all) eventually relapse, and the outcome of patients with MM after they become refractory, or ineligible to receive a proteasome inhibitor or an IMiD, is quite poor, with survival less than 1 year. Most late-line patients will become refractory to Pis, IMiDs, and anti-CD38 mAbs (triple-class refractory) with an estimated median overall survival (OS) of approximately 8-13 months.

Therefore, relapsed or refractory (R/R) MM, in particular, continues to constitute a significant unmet medical need, and novel therapeutic agents and treatments are needed.

### SUMMARY OF THE INVENTION

Provided herein are, inter alia, methods of treating a cancer (e.g., a B cell proliferative disorder, such as MM), and related compositions for use, uses, and articles of manufacture.

In one aspect, the invention features a method of treating a subject having a relapsed or refractory (R/R) multiple myeloma (MM), wherein the subject has previously received a B cell maturation factor (BCMA)-targeting therapeutic agent, the method including administering to the subject a bispecific antibody that binds to Fc receptor-homolog 5 (FcRH5) and cluster of differentiation 3 (CD3) in a dosing regimen including: (i) a first phase including administering the bispecific antibody to the subject in at least a first 21-day dosing cycle (C1), wherein the first phase includes administration of the bispecific antibody to the subject on (a) Day 1 of the C1; and (b) Day 2, Day 3, or Day 4 of the C1; and (ii) a second phase including one or more 21-day dosing cycles, wherein the second phase includes administering the bispecific antibody to the subject every three weeks (Q3W).

In another aspect, the invention features a method of treating a subject having an R/R MM including administering to the subject a bispecific antibody that binds to FcRH5 and CD3 in a dosing regimen including at least a first 21-day dosing cycle, wherein the first 21-day dosing cycle includes a first dose (C1D1), a second dose (C1D2), and a third dose (C1D3) of the bispecific antibody, wherein the C1D1 is between about 0.2 mg to about 0.4 mg and is administered to the subject on Day 1 of the first dosing cycle, the C1D2 is about 3.1 mg to about 3.4 mg and is administered to the subject on Day 2, Day 3, or Day 4 of the first dosing cycle, and the C1D3 is greater than the C1D2.

In another aspect, the invention features a method of treating a subject having an R/R MM, wherein the subject has a triple-class refractory MM and has previously received a BCMA-targeting TDB antibody, the method including administering to the subject a cevostamab monotherapy in a dosing regimen including: (i) a first phase including administering the cevostamab to the subject in a first dosing cycle (C1); and (ii) a second phase including administering the cevostamab to the subject every three weeks (Q3W), wherein each dosing cycle of the first and second phase is a 21-day dosing cycle, and cevostamab is administered to the subject: (i) at a first step-up dose of 0.3 mg during the first phase on Day 1 of the C1 and as a second step-up dose of 3.3 mg during the first phase on Day 2, Day 3, or Day 4 of the C1; (ii) at a target dose of 160 mg during the first phase on Day 8 of the C1; and (iii) at the target dose of 160 mg during the second phase on Day 1 of each dosing cycle. In some aspects, administration of the target dose of 160 mg may be delayed to on or after Day 9 of the C1 of the first phase instead of Day 8.

In another aspect, the invention features a method of treating a subject having an R/R MM, wherein the subject has a triple-class refractory MM and has previously received a BCMA-targeting TDB antibody, the method including administering to the subject a cevostamab monotherapy in a dosing regimen including: (i) a first phase including administering the cevostamab to the subject in a first dosing cycle (C1); and (ii) a second phase including administering the cevostamab to the subject Q3W, wherein each dosing cycle of the first and second phase is a 21-day dosing cycle, and cevostamab is administered to the subject: (i) at a first step-up dose of 0.3 mg during the first phase on Day 1 of the C1 and as a second step-up dose of 3.3 mg during the first phase on Day 2, Day 3, or Day 4 of the C1; (ii) at a target dose of 160 mg during the first phase on Day 8 of the C1; and (iii) at the target dose of 160 mg during the second phase on Day 1 of each dosing cycle. In some aspects, administration of the target dose of 160 mg may be delayed to on or after Day 9 of the C1 of the first phase instead of Day 8.

In another aspect, the invention features a method of treating a subject having an R/R MM, wherein the subject has a triple-class refractory MM and has previously received a BCMA-targeting CAR-T, the method including administering to the subject a cevostamab monotherapy in a dosing regimen including: (i) a first phase including administering the cevostamab to the subject in a first dosing cycle (C1); and (ii) a second phase including administering the cevostamab to the subject Q3W, wherein each dosing cycle of the first and second phase is a 21-day dosing cycle, and cevostamab is administered to the subject: (i) at a first step-up dose of 0.3 mg during the first phase on Day 1 of the C1 and as a second step-up dose of 3.3 mg during the first phase on Day 2, Day 3, or Day 4 of the C1; (ii) at a target dose of 160 mg during the first phase on Day 8 of the C1; and (iii) at the target dose of 160 mg during the second phase on Day 1 of each dosing cycle. In some aspects, administration of the target dose of 160 mg may be delayed to on or after Day 9 of the C1 of the first phase instead of Day 8.

In another aspect, the invention features a method of treating a subject having an R/R MM, wherein the subject has a triple-class refractory MM and has previously received a BCMA-targeting ADC, the method including administering to the subject a cevostamab monotherapy in a dosing regimen including: (i) a first phase including administering the cevostamab to the subject in a first dosing cycle (C1); and (ii) a second phase including administering the cevostamab to the subject Q3W, wherein each dosing cycle of the first and second phase is a 21-day dosing cycle, and cevostamab is administered to the subject: (i) at a first step-up dose of 0.3 mg during the first phase on Day 1 of the C1 and as a second step-up dose of 3.3 mg during the first phase on Day 2, Day 3, or Day 4 of the C1; (ii) at a target dose of 160 mg during the first phase on Day 8 of the C1; and (iii) at the target dose of 160 mg during the second phase on Day 1 of each dosing cycle. In some aspects, administration of the target dose of 160 mg may be delayed to on or after Day 9 of the C1 of the first phase instead of Day 8.

In another aspect, the invention features a bispecific antibody that binds to FcRH5 and CD3 for use in treatment of a subject having a R/R MM, wherein the subject has a triple class refractory MM and has previously received a BCMA-targeting therapeutic agent, the treatment including administration of the bispecific antibody to the subject in a dosing regimen including: (i) a first phase including a first 21-day dosing cycle (C1), wherein the first phase includes administering the bispecific antibody to the subject on (a) Day 1 of the C1; (b) Day 2, Day 3, or Day 4 of the C1; and (c) Day 8 (or on or after Day 9) of the C1; and (ii) a second phase including one or more 21-day dosing cycles, wherein the second phase includes administering the bispecific antibody to the subject Q3W.

In another aspect, the invention features a bispecific antibody that binds to FcRH5 and CD3 for use in treatment of a subject having an R/R MM including administering to the subject a bispecific antibody that binds to FcRH5 and CD3 in a dosing regimen including at least a first 21-day dosing cycle, wherein the first dosing cycle includes a first dose (C1D1), a second dose (C1D2), and a third dose (C1D3) of the bispecific antibody, wherein the C1D1 is between about 0.2 mg to about 0.4 mg and is administered to the subject on Day 1 of the first dosing cycle, the C1D2 is about 3.1 mg to about 3.4 mg and is administered to the subject on Day 2, Day 3, or Day 4 of the first dosing cycle, and the C1D3 is greater than the C1D2.

In some aspects, the subject has a triple-class refractory MM.

In some aspects, the BCMA-targeting therapeutic agent is selected from a BCMA-targeting T-cell-dependent bispecific (TDB) antibody, a BCMA-targeting antibody-drug conjugate (ADC), or a chimeric antigen receptor T (CAR-T).

In some aspects, the BCMA-targeting therapeutic agent is a BCMA-targeting TDB antibody.

In some aspects, the method further includes administering the bispecific antibody that binds to FcRH5 and CD3 to the subject during the first phase on Day 8 of the C1.

In some aspects, the method further includes administering the bispecific antibody that binds to FcRH5 and CD3 to the subject during the first phase on or after Day 9 of the C1.

In some aspects, the first phase includes administration of a first step-up dose and a second step-up dose of the bispecific antibody that binds to FcRH5 and CD3 to the subject.

In some aspects, the first step-up dose is administered to the subject on Day 1 of the C1 and the second step-up dose is administered to the subject on Day 2 of the C1.

In some aspects, (i) the first step-up dose is administered to the subject on Day 1 of the C1; (ii) the subject has a cytokine release syndrome (CRS) event following the first step-up dose; and (iii) the second step-up dose is administered to the subject on Day 3 of the C1 following a resolution of the CRS event.

In some aspects, (i) the first step-up dose is administered to the subject on Day 1 of the C1; (ii) the subject has a CRS event following the first step-up dose; and (iii) the second step-up dose is administered to the subject on Day 4 of the C1 following a resolution of the CRS event.

In some aspects, the first step-up dose is about 0.2% of a target dose and the second step-up dose is about 2% of the target dose.

In some aspects, the first step-up dose is about 0.3 mg and the second step-up dose is about 3.3 mg.

In some aspects, the target dose is administered to the subject on Day 8 of the C1.

In some aspects, the target dose is administered to the subject on or after Day 9 of the C1.

In some aspects, the first phase includes administration of a first step-up dose of the bispecific antibody that binds to FcRH5 and CD3 to the subject.

In some aspects, the first step-up dose is administered to the subject on Day 1 of the C1.

In some aspects, the first step-up dose is about 0.2% to about 2.3% of a target dose.

In some aspects, the first step-up dose is about 0.2% of a target dose.

In some aspects, the first step-up dose is about 2% of a target dose.

In some aspects, the first step-up dose is about 2.3% of a target dose.

In some aspects, the first step-up dose is about 0.3 mg to about 3.6 mg.

In some aspects, the first step-up dose is 0.3 mg.

In some aspects, the first step-up dose is 3.3 mg.

In some aspects, the first step-up dose is 3.6 mg.

In some aspects, the target dose is administered to the subject on Day 2 and Day 8 of the C1.

In some aspects, the target dose is administered to the subject on Day 2 and on or after Day 9 of the C1.

In some aspects, (i) the subject has a CRS event following the first step-up dose; (ii) the target dose is administered to the subject on Day 3 the C1 following a resolution of the CRS event; and (iii) the target dose is administered to the subject on Day 8 of the C1.

In some aspects, (i) the subject has a CRS event following the first step-up dose; (ii) the target dose is administered to the subject on Day 4 the C1 following a resolution of the CRS event; and (iii) the target dose is administered to the subject on Day 8 of the C1.

In some aspects, (i) the subject has a CRS event following the first step-up dose; (ii) the target dose is administered to the subject on Day 3 the C1 following a resolution of the CRS event; and (iii) the target dose is administered to the subject on or after Day 9 of the C1.

In some aspects, (i) the subject has a CRS event following the first step-up dose; (ii) the target dose is administered to the subject on Day 4 the C1 following a resolution of the CRS event; and (iii) the target dose is administered to the subject on or after Day 9 of the C1.

In some aspects, the second phase includes at least two dosing cycles, at least three dosing cycles, at least four dosing cycles, at least five dosing cycles, at least six dosing cycles, at least seven dosing cycles, at least eight dosing cycles, at least nine dosing cycles, at least ten dosing cycles, at least eleven dosing cycles, at least twelve dosing cycles, or at least thirteen dosing cycles.

In some aspects, the second phase includes a first dosing cycle (C1), a second dosing cycle (C2), a third dosing cycle (C3), and a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), and/or a thirteenth dosing cycle (C13).

In some aspects, the second phase includes administration of the bispecific antibody that binds to FcRH5 and CD3 to the subject on Day 1 of each dosing cycle.

In some aspects, the second phase includes a C1, and Day 1 of the C1 of the second phase is at least 7 days after administration of a target dose of the bispecific antibody in the first phase.

In some aspects, a target dose of the bispecific antibody that binds to FcRH5 and CD3 is administered to the subject for each administration during the second phase.

In some aspects, the second phase includes administering the bispecific antibody that binds to FcRH5 and CD3 to the subject Q3W until the subject experiences disease progression, unacceptable toxicity, or death.

In some aspects, the target dose is 160 mg.

In some aspects, the bispecific antibody that binds to FcRH5 and CD3 is administered to the subject as a monotherapy.

In some aspects, the bispecific antibody that binds to FcRH5 and CD3 is administered to the subject intravenously.

In some aspects, the bispecific antibody that binds to FcRH5 and CD3 includes an anti-FcRH5 arm including a first binding domain including the following six hypervariable regions (HVRs): (i) an HVR-H1 including the amino acid sequence of RFGVH (SEQ ID NO: 1); (ii) an HVR-H2 including the amino acid sequence of VIWRGGSTDYNAAFVS (SEQ ID NO: 2); (iii) an HVR-H3 including the amino acid sequence of HYYGSSDYALDN (SEQ ID NO:3); (iv) an HVR-L1 including the amino acid sequence of KASQDVRNLVV (SEQ ID NO: 4); (v) an HVR-L2 including the amino acid sequence of SGSYRYS (SEQ ID NO: 5); and (vi) an HVR-L3 including the amino acid sequence of QQHYSPPYT (SEQ ID NO: 6).

In some aspects, the bispecific antibody that binds to FcRH5 and CD3 includes an anti-FcRH5 arm including a first binding domain including (i) a heavy chain variable (VH) domain including an amino acid sequence having at least 95% sequence identity to the amino acid sequence of SEQ ID NO: 7; (ii) a light chain variable (VL) domain including an amino acid sequence having at least 95% sequence identity to the amino acid sequence of SEQ ID NO: 8; or (iii) a VH domain as in (i) and a VL domain as in (ii).

In some aspects, the first binding domain includes a VH domain including an amino acid sequence of SEQ ID NO: 7 and a VL domain including an amino acid sequence of SEQ ID NO: 8.

In some aspects, the bispecific antibody that binds to FcRH5 and CD3 includes an anti-CD3 arm including a second binding domain including the following six HVRs: (i) an HVR-H1 including the amino acid sequence of SYYIH (SEQ ID NO: 9); (ii) an HVR-H2 including the amino acid sequence of WIYPENDNTKYNEKFKD (SEQ ID NO: 10); (iii) an HVR-H3 including the amino acid sequence of DGYSRYYFDY (SEQ ID NO: 11); (iv) an HVR-L1 including the amino acid sequence of KSSQSLLNSRTRKNYLA (SEQ ID NO: 12); (v) an HVR-L2 including the amino acid sequence of WTSTRKS (SEQ ID NO: 13); and (vi) an HVR-L3 including the amino acid sequence of KQSFILRT (SEQ ID NO: 14).

In some aspects, the bispecific antibody that binds to FcRH5 and CD3 includes an anti-CD3 arm including a second binding domain including (i) a VH domain including an amino acid sequence having at least 95% sequence identity to the amino acid sequence of SEQ ID NO: 15; (ii) a VL domain including an amino acid sequence having at least 95% sequence identity to the amino acid sequence of SEQ ID NO: 16; or (iii) a VH domain as in (i) and a VL domain as in (ii).

In some aspects, the second binding domain includes a VH domain including an amino acid sequence of SEQ ID NO: 15 and a VL domain including an amino acid sequence of SEQ ID NO: 16.

In some aspects, the bispecific antibody that binds to FcRH5 and CD3 includes an anti-FcRH5 arm including a heavy chain polypeptide (H1) and a light chain polypeptide (L1) and an anti-CD3 arm including a heavy chain polypeptide (H2) and a light chain polypeptide (L2), and wherein: (i) H1 includes the amino acid sequence of SEQ ID NO: 35; (ii) L1 includes the amino acid sequence of SEQ ID NO: 36; (iii) H2 includes the amino acid sequence of SEQ ID NO: 37; and (iv) L2 includes the amino acid sequence of SEQ ID NO: 38.

In some aspects, the bispecific antibody that binds to FcRH5 and CD3 includes an aglycosylation site mutation.

In some aspects, the aglycosylation site mutation reduces effector function of the bispecific antibody.

In some aspects, the aglycosylation site mutation is a substitution mutation.

In some aspects, the bispecific antibody that binds to FcRH5 and CD3 includes a substitution mutation in the Fc region that reduces effector function.

In some aspects, the bispecific antibody that binds to FcRH5 and CD3 is a monoclonal antibody.

In some aspects, the bispecific antibody that binds to FcRH5 and CD3 is a humanized antibody.

In some aspects, the bispecific antibody that binds to FcRH5 and CD3 is a chimeric antibody.

In some aspects, the bispecific antibody that binds to FcRH5 and CD3 is an antibody fragment that binds FcRH5 and CD3.

In some aspects, the antibody fragment is selected from the group consisting of Fab, Fab′-SH, Fv, scFv, and (Fab′)₂ fragments.

In some aspects, the bispecific antibody that binds to FcRH5 and CD3 is a full-length antibody.

In some aspects, the bispecific antibody that binds to FcRH5 and CD3 is an IgG antibody.

In some aspects, the IgG antibody is an IgG1 antibody.

In some aspects, the bispecific antibody that binds to FcRH5 and CD3 includes one or more heavy chain constant domains, wherein the one or more heavy chain constant domains are selected from a first CH1 (CH1₁) domain, a first CH2 (CH2₁) domain, a first CH3 (CH3₁) domain, a second CH1 (CH1₂) domain, second CH2 (CH2₂) domain, and a second CH3 (CH32) domain.

In some aspects, at least one of the one or more heavy chain constant domains is paired with another heavy chain constant domain.

In some aspects, the CH3₁ and CH3₂ domains each include a protuberance or cavity, and wherein the protuberance or cavity in the CH3₁ domain is positionable in the cavity or protuberance, respectively, in the CH3₂ domain.

In some aspects, the CH3₁ and CH3₂ domains meet at an interface between the protuberance and cavity.

In some aspects, the CH2₁ and CH2₂ domains each include a protuberance or cavity, and wherein the protuberance or cavity in the CH2₁ domain is positionable in the cavity or protuberance, respectively, in the CH2₂ domain.

In some aspects, the CH2₁ and CH2₂ domains meet at an interface between said protuberance and cavity.

In some aspects, the anti-FcRH5 arm includes the protuberance and the anti-CD3 arm includes the cavity.

In some aspects, a CH3 domain of the anti-FcRH5 arm includes a protuberance including a T366W amino acid substitution mutation (EU numbering) and a CH3 domain of the anti-CD3 arm includes a cavity including T366S, L368A, and Y407V amino acid substitution mutations (EU numbering).

In some aspects, the bispecific antibody that binds to FcRH5 and CD3 is cevostamab.

In some aspects, the cevostamab is administered as a monotherapy.

In some aspects, the bispecific antibody that binds to FcRH5 and CD3 is administered to the subject concurrently with one or more additional therapeutic agents.

In some aspects, the bispecific antibody that binds to FcRH5 and CD3 is administered to the subject prior to the administration of one or more additional therapeutic agents.

In some aspects, the bispecific antibody that binds to FcRH5 and CD3 is administered to the subject subsequent to the administration of one or more additional therapeutic agents.

In some aspects, the one or more additional therapeutic agents include an effective amount of tocilizumab.

In some aspects, tocilizumab is administered to the subject by intravenous infusion.

In some aspects, (i) the subject weighs ≥30 kg, and tocilizumab is administered to the subject at a dose of 8 mg/kg; or (ii) the subject weighs <30 kg, and tocilizumab is administered to the subject at a dose of 12 mg/kg, wherein the tocilizumab is administered to the subject at a dose that does not exceed 800 mg.

In some aspects, tocilizumab is administered to the subject 2 hours before administration of the bispecific antibody.

In some aspects, the one or more additional therapeutic agents include an effective amount of a BCMA-directed therapeutic agent.

In some aspects, the subject has a CRS event, and the method further includes treating the symptoms of the CRS event while suspending treatment with the bispecific antibody that binds to FcRH5 and CD3.

In some aspects, the method further includes treating the symptoms of the CRS event.

In some aspects, treating the symptoms of the CRS event includes administering to the subject an effective amount of tocilizumab.

In some aspects, tocilizumab is administered intravenously to the subject as a single dose of about 8 mg/kg.

In some aspects, the CRS event does not resolve or worsens within 24 hours of treating the symptoms of the CRS event, and the method further includes administering to the subject one or more additional doses of tocilizumab to manage the CRS event.

In some aspects, the one or more additional doses of tocilizumab are administered intravenously to the subject at a dose of about 8 mg/kg.

In some aspects, the one or more additional therapeutic agents include an effective amount of acetaminophen or paracetamol.

In some aspects, acetaminophen or paracetamol is administered to the subject at a dose of between about 500 mg to about 1000 mg.

In some aspects, acetaminophen or paracetamol is administered to the subject orally.

In some aspects, the one or more additional therapeutic agents include an effective amount of diphenhydramine.

In some aspects, diphenhydramine is administered to the subject at a dose of between about 25 mg to about 50 mg.

In some aspects, diphenhydramine is administered orally to the subject.

In some aspects, the method includes premedication with the following agents prior to administration of the bispecific antibody to the subject: (i) a corticosteroid; (ii) acetaminophen or paracetamol; and/or (iii) diphenhydramine.

In some aspects, the corticosteroid is administered to the subject 1 hour (±15 minutes) prior to any administration of the bispecific antibody during the first phase.

In some aspects, the subject has experienced CRS with a prior administration of the bispecific antibody and the corticosteroid is administered to the subject 1 hour (±15 minutes) prior to any administration of the bispecific antibody during the second phase.

In some aspects, the corticosteroid is administered to the subject 24 hours prior to any administration of the bispecific antibody during the first phase.

In some aspects, the corticosteroid is dexamethasone or methylprednisolone.

In some aspects, the corticosteroid is dexamethasone.

In some aspects, the dexamethasone is administered to the subject at a dose of about 20 mg.

In some aspects, the methylprednisolone is administered to the subject at a dose of about 80 mg.

In some aspects, the corticosteroid is administered to the subject intravenously.

In some aspects, acetaminophen or paracetamol is administered to the subject at a dose of between 500 mg to 1000 mg.

In some aspects, acetaminophen or paracetamol is administered to the subject orally.

In some aspects, diphenhydramine is administered to the subject at a dose of between 25 mg to 50 mg.

In some aspects, diphenhydramine is administered to the subject orally.

In some aspects, the subject has received at least four prior lines of treatment for the MM.

In some aspects, the subject has been exposed to a prior treatment including a proteasome inhibitor (PI), an IMiD, an anti-CD38 therapeutic agent, and/or an autologous stem cell transplant (ASCT).

In some aspects, the PI is bortezomib, carfilzomib, or ixazomib.

In some aspects, the IMiD is thalidomide, lenalidomide, or pomalidomide.

In some aspects, the anti-CD38 therapeutic agent is an anti-CD38 antibody.

In some aspects, the anti-CD38 antibody is daratumumab, MOR202, or isatuximab.

In some aspects, the anti-CD38 antibody is daratumumab.

In some aspects, the BCMA-targeting TDB antibody is teclistimab (JNJ-64007957) AM701, AMG 420, CC-93269, elranatamab, TNB-383B, linvoseltamab (REGN5458), alnuctamab (CC-93269), AFM26, or HPN217.

In some aspects, the BCMA-targeting antibody-drug conjugate (ADC) is BLENREP® (belantamab mafodotin).

In some aspects, the chimeric antigen receptor T (CAR-T) is selected from ABECMA® (idecabtagene-vicleucel) and CARVYKTI® (ciltacabtagene autoleucel).

### BRIEF DESCRIPTION OF THE DRAWING

FIG. 1 shows a study schema for the CAMMA 2 (CO43476) study described in Example 1. In brief, the study involves relapsed or refractory (R/R) multiple myeloma (MM) subjects that have previously received a B cell maturation factor (BCMA)-targeting therapy (e.g., a BCMA-targeting T-cell-dependent bispecific (TDB) antibody, a BCMA-targeting antibody-drug conjugate (ADC), or a chimeric antigen receptor T (CAR-T)-cell) and are triple-class refractory to, for example, a proteosome inhibitor (PI), an immunomodulatory drug (IMiD), and an anti-cluster of differentiation 38 (CD38) antibody. In an initial exploratory cohort (Cohort A), the R/R MM subjects are intravenously (IV) administered cevostamab at a first step-up dose of 0.3 mg, followed by a second step-up dose of 3.3 mg, followed by a target dose of 160 mg. The target dose of 160 mg may then be administered every three weeks (Q3W). In a larger expansion cohort (Cohort B), the R/R MM subjects are administered a cevostamab monotherapy at a recommended Phase 2 dose (RP2D). For cohorts A and B, treatment will continue until disease progression, unacceptable toxicity, or death, whichever occurs first.

### DETAILED DESCRIPTION OF THE INVENTION

### I. Definitions

The term “about” as used herein refers to the usual error range for the respective value readily known to the skilled person in this technical field. Reference to “about” a value or parameter herein includes (and describes) aspects that are directed to that value or parameter per se.

It is understood that aspects of the invention described herein include “comprising,” “consisting,” and “consisting essentially of” aspects.

The term “FcRH5” or “fragment crystallizable receptor-like 5,” as used herein, refers to any native FcRH5 from any vertebrate source, including mammals such as primates (e.g., humans) and rodents (e.g., mice and rats), unless otherwise indicated, and encompasses “full-length,” unprocessed FcRH5, as well as any form of FcRH5 that results from processing in the cell. The term also encompasses naturally occurring variants of FcRH5, including, for example, splice variants or allelic variants. FcRH5 includes, for example, human FcRH5 protein (UniProtKB/Swiss-Prot ID: Q96RD9.3), which is 977 amino acids in length.

The terms “anti-FcRH5 antibody” and “an antibody that binds to FcRH5” refer to an antibody that is capable of binding FcRH5 with sufficient affinity such that the antibody is useful as a diagnostic and/or therapeutic agent in targeting FcRH5. In one embodiment, the extent of binding of an anti-FcRH5 antibody to an unrelated, non-FcRH5 protein is less than about 10% of the binding of the antibody to FcRH5 as measured, e.g., by a radioimmunoassay (RIA). In certain embodiments, an antibody that binds to FcRH5 has a dissociation constant (K_{D}) of: ≤1 μM, ≤250 nM, ≤100 nM, ≤15 nM, ≤10 nM, ≤6 nM, ≤4 nM, ≤2 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain embodiments, an anti-FcRH5 antibody binds to an epitope of FcRH5 that is conserved among FcRH5 from different species.

The term “cluster of differentiation 3” or “CD3,” as used herein, refers to any native CD3 from any vertebrate source, including mammals such as primates (e.g., humans) and rodents (e.g., mice and rats), unless otherwise indicated, including, for example, CD3ε, CD3γ, CD3α, and CD3β chains. The term encompasses “full-length,” unprocessed CD3 (e.g., unprocessed or unmodified CD3ε or CD3γ), as well as any form of CD3 that results from processing in the cell. The term also encompasses naturally occurring variants of CD3, including, for example, splice variants or allelic variants. CD3 includes, for example, human CD3ε protein (NCBI RefSeq No. NP_000724), which is 207 amino acids in length, and human CD3γ protein (NCBI RefSeq No. NP_000064), which is 182 amino acids in length.

The terms “anti-CD3 antibody” and “an antibody that binds to CD3” refer to an antibody that is capable of binding CD3 with sufficient affinity such that the antibody is useful as a diagnostic and/or therapeutic agent in targeting CD3. In one embodiment, the extent of binding of an anti-CD3 antibody to an unrelated, non-CD3 protein is less than about 10% of the binding of the antibody to CD3 as measured, e.g., by a radioimmunoassay (RIA). In certain embodiments, an antibody that binds to CD3 has a dissociation constant (K_{D}) of ≤1 μM, ≤250 nM, ≤100 nM, ≤15 nM, ≤10 nM, ≤5 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain embodiments, an anti-CD3 antibody binds to an epitope of CD3 that is conserved among CD3 from different species.

For the purposes herein, “cevostamab,” also referred to as BFCR4350A or RO7187797, is an Fc-engineered, humanized, full-length non-glycosylated IgG1 kappa T-cell-dependent bispecific antibody (TDB) that binds FcRH5 and CD3 and comprises an anti-FcRH5 arm comprising the heavy chain polypeptide sequence of SEQ ID NO: 35 and the light chain polypeptide sequence of SEQ ID NO: 36 and an anti-CD3 arm comprising the heavy chain polypeptide sequence of SEQ ID NO: 37 and the light chain polypeptide sequence of SEQ ID NO: 38. Cevostamab comprises a threonine to tryptophan amino acid substitution at position 366 on the heavy chain of the anti-FcRH5 arm (T366W) using EU numbering of Fc region amino acid residues and three amino acid substitutions (tyrosine to valine at position 407, threonine to serine at position 366, and leucine to alanine at position 368) on the heavy chain of the anti-CD3 arm (Y407V, T366S, and L368A) using EU numbering of Fc region amino acid residues to drive heterodimerization of the two arms (half-antibodies). Cevostamab also comprises an amino acid substitution (asparagine to glycine) at position 297 on each heavy chain (N297G) using EU numbering of Fc region amino acid residues, which results in a non-glycosylated antibody that has minimal binding to Fc (Fcγ) receptors and, consequently, prevents Fc-effector function. Cevostamab is also described in WHO Drug Information (International Nonproprietary Names for Pharmaceutical Substances), Recommended INN: List 84, Vol. 34, No. 3, published 2020 (see page 701).

The term “antibody” herein is used in the broadest sense and encompasses various antibody structures, including but not limited to monoclonal antibodies, polyclonal antibodies, multispecific antibodies (e.g., bispecific antibodies), and antibody fragments (e.g., bis-Fabs) so long as they exhibit the desired antigen-binding activity.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary aspects for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

With regard to the binding of an antibody to a target molecule, the term “binds” or “binding” or “specifically binds” or is “specific for” a particular polypeptide or an epitope on a particular polypeptide target means binding that is measurably different from a non-specific interaction. Specific binding can be measured, for example, by determining binding of a molecule compared to binding of a control molecule. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “binds” or “binding” or “specifically binds to” or is “specific for” a particular polypeptide or an epitope on a particular polypeptide target as used herein can be exhibited, for example, by a molecule having a K_{D} for the target of 10⁻⁴ M or lower, alternatively 10⁻⁵ M or lower, alternatively 10⁻⁶ M or lower, alternatively 10⁻⁷ M or lower, alternatively 10⁻⁸ M or lower, alternatively 10⁻⁹ M or lower, alternatively 10⁻¹⁰ M or lower, alternatively 10⁻¹¹ M or lower, alternatively 10⁻¹² M or lower or a K_{D} in the range of 10⁻⁴ M to 10⁻⁶ M or 10⁻⁶ M to 10⁻¹⁰ M or 10⁻⁷ M to 10⁻⁹ M. As will be appreciated by the skilled artisan, affinity and K_{D} values are inversely related. A high affinity for an antigen is measured by a low K_{D} value. In one embodiment, the term “binding” refers to binding where a molecule binds to a particular polypeptide or epitope on a particular polypeptide without substantially binding to any other polypeptide or polypeptide epitope.

The terms “full-length antibody,” “intact antibody,” and “whole antibody” are used herein interchangeably to refer to an antibody having a structure substantially similar to a native antibody structure or having heavy chains that contain an Fc region as defined herein.

An “antibody fragment” refers to a molecule other than an intact antibody that comprises a portion of an intact antibody that binds the antigen to which the intact antibody binds. Examples of antibody fragments include but are not limited to bis-Fabs; Fv; Fab; Fab′-SH; F(ab′)₂; diabodies; linear antibodies; single-chain antibody molecules (e.g., scFv, ScFab); and multispecific antibodies formed from antibody fragments.

A “single-domain antibody” refers to an antibody fragment comprising all or a portion of the heavy chain variable domain or all or a portion of the light chain variable domain of an antibody. In certain aspects, a single-domain antibody is a human single-domain antibody (see, e.g., U.S. Pat. No. 6,248,516 B1). Examples of single-domain antibodies include but are not limited to a VHH.

A “Fab” fragment is an antigen-binding fragment generated by papain digestion of antibodies and consists of an entire L chain along with the variable region domain of the H chain (VH), and the first constant domain of one heavy chain (CH1). Papain digestion of antibodies produces two identical Fab fragments. Pepsin treatment of an antibody yields a single large F(ab′)₂ fragment which roughly corresponds to two disulfide linked Fab fragments having divalent antigen-binding activity and is still capable of cross-linking antigen. Fab′ fragments differ from Fab fragments by having an additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

“Fv” consists of a dimer of one heavy- and one light-chain variable region domain in tight, noncovalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although often at a lower affinity than the entire binding site.

The term “Fc region” herein is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all Lys447 residues removed, antibody populations with no Lys447 residues removed, and antibody populations having a mixture of antibodies with and without the Lys447 residue.

A “functional Fc region” possesses an “effector function” of a native sequence Fc region. Exemplary “effector functions” include C1q binding; complement-dependent cytotoxicity (CDC); Fc receptor binding; antibody-dependent cell-mediated cytotoxicity (ADCC); phagocytosis; down regulation of cell surface receptors (e.g., B cell receptor; BCR); B cell activation, etc. Such effector functions generally require the Fc region to be combined with a binding domain (e.g., an antibody variable domain) and can be assessed using various assays as disclosed, for example, in definitions herein.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region, as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g., from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, preferably at least about 90% homology therewith, or preferably at least about 95% homology therewith.

“Fc complex” as used herein refers to CH3 domains of two Fc regions interacting together to form a dimer or, as in certain aspects, two Fc regions interact to form a dimer, wherein the cysteine residues in the hinge regions and/or the CH3 domains interact through bonds and/or forces (e.g., Van der Waals, hydrophobic forces, hydrogen bonds, electrostatic forces, or disulfide bonds).

“Hinge region” is generally defined as stretching from about residue 216 to 230 of an IgG (EU numbering), from about residue 226 to 243 of an IgG (Kabat numbering), or from about residue 1 to 15 of an IgG (IMGT unique numbering).

The “lower hinge region” of an Fc region is normally defined as the stretch of residues immediately C-terminal to the hinge region, i.e., residues 233 to 239 of the Fc region (EU numbering).

“Fc receptor” or “FcR” describes a receptor that binds to the Fc region of an antibody. A preferred FcR is a native sequence human FcR. Moreover, a preferred FcR is one that binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see review M. in Daëron, Annu. Rev. Immunol. 15:203-234 (1997)). FcRs are reviewed in Ravetch and Kinet, Annu. Rev. Immunol. 9:457-492 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

The term “knob-into-hole” or “KnH” technology as mentioned herein refers to the technology directing the pairing of two polypeptides together in vitro or in vivo by introducing a protuberance (knob) into one polypeptide and a cavity (hole) into the other polypeptide at an interface in which they interact. For example, KnHs have been introduced in the Fc:Fc interaction interfaces, CL:CH1 interfaces or VH/VL interfaces of antibodies (e.g., US2007/0178552, WO 96/027011, WO 98/050431 and Zhu et al., (1997) Protein Science 6:781-788). This is especially useful in driving the pairing of two different heavy chains together during the manufacture of multispecific antibodies. For example, multispecific antibodies having KnH in their Fc regions can further comprise single variable domains linked to each Fc region, or further comprise different heavy chain variable domains that pair with identical, similar, or different light chain variable domains. KnH technology can also be used to pair two different receptor extracellular domains together or any other polypeptide sequences that comprise different target recognition sequences.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The “CH1 region” or “CH1 domain” comprises the stretch of residues from about residue 118 to residue 215 of an IgG (EU numbering), from about residue 114 to 223 of an IgG (Kabat numbering), or from about residue 1.4 to residue 121 of an IgG (IMGT unique numbering) (Lefranc et al., IMGT®, the international ImMunoGeneTics information system® 25 years on. Nucleic Acids Res. 2015 January; 43(Database issue):D413-22).

The “CH2 domain” of a human IgG Fc region usually extends from about residues 244 to about 360 of an IgG (Kabat numbering), from about residues 231 to about 340 of an IgG (EU numbering), or from about residues 1.6 to about 125 of an IgG (IGMT unique numbering). The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Molec. Immunol. 22:161-206 (1985).

The “CH3 domain” comprises the stretch of residues C-terminal to a CH2 domain in an Fc region (i.e., from about amino acid residue 361 to about amino acid residue 478 of an IgG (Kabat numbering), from about amino acid residue 341 to about amino acid residue 447 of an IgG (EU numbering), or from about amino acid residue 1.4 to about amino acid residue 130 of an IgG (IGMT unique numbering)).

The “CL domain” or “constant light domain” comprises the stretch of residues C-terminal to a light-chain variable domain (VL). The light chain (LC) of an antibody may be a kappa (κ) (“Cκ”) or lambda (λ) (“Cλ”) light chain region. The Cκ region generally extends from about residue 108 to residue 214 of an IgG (Kabat or EU numbering) or from about residue 1.4 to residue 126 of an IgG (IMGT unique numbering). The Cλ residue generally extends from about residue 107a to residue 215 (Kabat numbering) or from about residue 1.5 to residue 127 (IMGT unique numbering) (Lefranc et al., supra).

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. Human antibodies can be produced using various techniques known in the art, including phage-display libraries. Hoogenboom and Winter, J. Mol. Biol. 227:381,1991; Marks et al., J. Mol. Biol. 222:581, 1991. Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95, 1991. See also van Dijk and van de Winkel, Curr. Opin. Pharmacol. 5:368-74, 2001. Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA. 103:3557-3562, 2006, regarding human antibodies generated via a human B-cell hybridoma technology.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al. Sequences of Proteins of Immunological Interest, Fifth Edition, NIH Publication 91-3242, Bethesda MD (1991), vols. 1-3. In one aspect, for the VL, the subgroup is subgroup kappa I as in Kabat et al. supra. In one aspect, for the VH, the subgroup is subgroup III as in Kabat et al. supra.

A “humanized” antibody refers to a chimeric antibody comprising amino acid residues from non-human HVRs and amino acid residues from human FRs. In certain aspects, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. In certain aspects in which all or substantially all of the FRs of a humanized antibody correspond to those of a human antibody, any of the FRs of the humanized antibody may contain one or more amino acid residues (e.g., one or more Vernier position residues of FRs) from non-human FR(s). A humanized antibody optionally may comprise at least a portion of an antibody constant region derived from a human antibody. A “humanized form” of an antibody, e.g., a non-human antibody, refers to an antibody that has undergone humanization.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al., Kuby Immunology, 6^{th} ed. W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887, 1993; Clarkson et al. Nature 352:624-628, 1991.

The term “hypervariable region” or “HVR” as used herein refers to each of the regions of an antibody variable domain which are hypervariable in sequence (“complementarity determining regions” or “CDRs”). Generally, antibodies comprise six CDRs: three in the VH (CDR-H1, CDR-H2, CDR-H3), and three in the VL (CDR-L1, CDR-L2, CDR-L3). Exemplary CDRs herein include:
(a) CDRs occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917, 1987);
(b) CDRs occurring at amino acid residues 24-34 (L1), 50-56 (L2), 89-97 (L3), 31-35b (H1), 50-65 (H2), and 95-102 (H3) (Kabat et al. Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745, 1996).

Unless otherwise indicated, HVR residues and other residues in the variable domain (e.g., FR residues) are numbered herein according to Kabat et al. supra.

“Single-chain Fv” also abbreviated as “sFv” or “scFv” are antibody fragments that comprise the VH and VL antibody domains connected into a single polypeptide chain. Preferably, the scFv polypeptide further comprises a polypeptide linker between the VH and VL domains, which enables the scFv to form the desired structure for antigen binding. For a review of scFv, see Pluckthün, The Pharmacology of Monoclonal Antibodies, vol. 113, Rosenburg and Moore eds., Springer-Verlag, New York, pp. 269-315 (1994); Malmborg et al., J. Immunol. Methods 183:7-13, 1995.

By “targeting domain” is meant a part of a compound or a molecule that specifically binds to a target epitope, antigen, ligand, or receptor. Targeting domains include but are not limited to antibodies (e.g., monoclonal, polyclonal, recombinant, humanized, and chimeric antibodies), antibody fragments or portions thereof (e.g., bis-Fab fragments, Fab fragments, F(ab′)₂, scFab, scFv antibodies, SMIP, single-domain antibodies, diabodies, minibodies, scFv-Fc, affibodies, nanobodies, and VH and/or VL domains of antibodies), receptors, ligands, aptamers, peptide targeting domains (e.g., cysteine knot proteins (CKP)), and other molecules having an identified binding partner. A targeting domain may target, block, agonize, or antagonize the antigen to which it binds.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “multispecific antibody” is used in the broadest sense and specifically covers an antibody that has polyepitopic specificity. In one aspect, the multispecific antibody binds to two different targets (e.g., bispecific antibody). Such multispecific antibodies include, but are not limited to, an antibody comprising a heavy chain variable domain (VH) and a light chain variable domain (VL), where the VH/VL unit has polyepitopic specificity, antibodies having two or more VL and VH domains with each VH/VL unit binding to a different epitope, antibodies having two or more single variable domains with each single variable domain binding to a different epitope, full-length antibodies, antibody fragments such as Fab, Fv, dsFv, scFv, diabodies, bispecific diabodies and triabodies, antibody fragments that have been linked covalently or non-covalently. “Polyepitopic specificity” refers to the ability to specifically bind to two or more different epitopes on the same or different target(s). “Monospecific” refers to the ability to bind only one antigen. In one aspect, the monospecific biepitopic antibody binds two different epitopes on the same target/antigen. In one aspect, the monospecific polyepitopic antibody binds to multiple different epitopes of the same target/antigen. According to one aspect, the multispecific antibody is an IgG antibody that binds to each epitope with an affinity of 5 μM to 0.001 pM, 3 μM to 0.001 pM, 1 μM to 0.001 pM, 0.5 μM to 0.001 pM, or 0.1 μM to 0.001 pM.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 Daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

As used herein, the term “immunoadhesin” designates molecules which combine the binding specificity of a heterologous protein (an “adhesin”) with the effector functions of immunoglobulin constant domains. Structurally, the immunoadhesins comprise a fusion of an amino acid sequence with a desired binding specificity, which amino acid sequence is other than the antigen recognition and binding site of an antibody (i.e., is “heterologous” compared to a constant region of an antibody), and an immunoglobulin constant domain sequence (e.g., CH2 and/or CH3 sequence of an IgG). The adhesin and immunoglobulin constant domains may optionally be separated by an amino acid spacer. Exemplary adhesin sequences include contiguous amino acid sequences that comprise a portion of a receptor or a ligand that binds to a protein of interest. Adhesin sequences can also be sequences that bind a protein of interest, but are not receptor or ligand sequences (e.g., adhesin sequences in peptibodies). Such polypeptide sequences can be selected or identified by various methods, include phage display techniques and high throughput sorting methods. The immunoglobulin constant domain sequence in the immunoadhesin can be obtained from any immunoglobulin, such as IgG1, IgG2, IgG3, or IgG4 subtypes, IgA (including IgA1 and IgA2), IgE, IgD, or IgM.

“Chemotherapeutic agent” includes chemical compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, rapamycin (Sirolimus, RAPAMUNE®, Wyeth), Lapatinib (TYKERB®, GSK572016, Glaxo Smith Kline), lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ω1I (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Chemotherapeutic agent also includes (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rHL-2; a topoisomerase 1 inhibitor such as LURTOTECAN®; ABARELIX® rmRH; and (ix) pharmaceutically acceptable salts, acids and derivatives of any of the above.

Chemotherapeutic agent also includes antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG1 A antibody genetically modified to recognize interleukin-12 p40 protein.

Chemotherapeutic agent also includes “EGFR inhibitors,” which refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see U.S. Pat. No. 4,943,533) and variants thereof, such as chimerized 225 (C225 or Cetuximab; ERBUTIX®) and reshaped human 225 (H225) (see WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al., Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6. 3 and E7.6. 3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine).

Chemotherapeutic agents also include “tyrosine kinase inhibitors” including the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFα) blockers such as etanercept (Enbrel), infliximab (Remicade), adalimumab (Humira), certolizumab pegol (Cimzia), golimumab (Simponi), interleukin 1 (IL-1) blockers such as anakinra (Kineret), T cell costimulation blockers such as abatacept (Orencia), interleukin 6 (IL-6) blockers such as tocilizumab (ACTEMRA®); interleukin 13 (IL-13) blockers such as lebrikizumab; interferon alpha (IFN) blockers such as Rontalizumab; beta 7 integrin blockers such as rhuMAb Beta7; IgE pathway blockers such as Anti-M1 prime; Secreted homotrimeric LTa3 and membrane bound heterotrimer LTa1/β2 blockers such as anti-lymphotoxin alpha (LTa); radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹², and radioactive isotopes of Lu); miscellaneous investigational agents such as thioplatin, PS-341, phenylbutyrate, ET-18-OCH3, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g. celecoxib or etoricoxib), proteosome inhibitor (e.g. PS341); CCI-779; tipifarnib (R11577); orafenib, ABT510; Bcl-2 inhibitor such as oblimersen sodium (GENASENSE®); pixantrone; farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents also include non-steroidal anti-inflammatory drugs with analgesic, antipyretic and anti-inflammatory effects. NSAIDs include non-selective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lornoxicam and isoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, and valdecoxib. NSAIDs can be indicated for the symptomatic relief of conditions such as rheumatoid arthritis, osteoarthritis, inflammatory arthropathies, ankylosing spondylitis, psoriatic arthritis, Reiter's syndrome, acute gout, dysmenorrhea, metastatic bone pain, headache and migraine, postoperative pain, mild-to-moderate pain due to inflammation and tissue injury, pyrexia, ileus, and renal colic.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹², and radioactive isotopes of Lu); chemotherapeutic agents or drugs (e.g., methotrexate, doxorubicin (ADRIAMYCIN®), vinca alkaloids (vincristine, vinblastine, etoposide), melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

A “disorder” is any condition that would benefit from treatment including, but not limited to, chronic and acute disorders or diseases including those pathological conditions which predispose a mammal to the disorder in question. In one aspect, the disorder is a cancer, e.g., a B cell proliferative disorder such as an MM, e.g., relapsed or refractory MM.

The terms “cell proliferative disorder” and “proliferative disorder” refer to disorders that are associated with some degree of abnormal cell proliferation. In one aspect, the cell proliferative disorder is cancer. In one aspect, the cell proliferative disorder is a tumor.

“Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

The terms “cancer” and “cancerous” refer to or describe the physiological condition in mammals that is typically characterized by unregulated cell growth/proliferation. Aspects of cancer include solid tumor cancers and non-solid tumor cancers. Examples of cancer include, but are not limited to, B cell proliferative disorders, such as MM, which may be relapsed or refractory MM. The MM may be, e.g., typical MM (e.g., immunoglobulin G (IgG) MM, IgA MM, IgD MM, IgE MM, or IgM MM), light chain MM (LCMM) (e.g., lambda light chain MM or kappa light chain MM), or non-secretory MM. The MM may have one or more cytogenetic features (e.g., high-risk cytogenic features), e.g., t(4;14), t(11;14), t(14;16), and/or del(17 p), as described in Table 1 and in the International Myeloma Working Group (IMWG) criteria provided in Sonneveld et al., Blood, 127(24): 2955-2962, 2016, and/or 1q21, as described in Chang et al., Bone Marrow Transplantation, 45: 117-121, 2010. Cytogenic features may be detected, e.g., using fluorescent in situ hybridization (FISH).

TABLE 1
Cytogenic features of MM

| Primary genetic events | | Secondary genetic events | |
|---|---|---|---|
| IgH translocation | Gene(s) | Deletion | Gene(s) |
| t(4; 14) | FGFR3/MMSET | 1p | CDKN2C, FAF1, FAM46C |
| t(6; 14) | CCND3 | 6q | |
| t(11; 14) | CCND1 | 8p | |
| t(14; 16) | MAF | 13 | RB1, DIS3 |
| t(14; 20) | MAFB | 11q | BIRC2/BIRC3 |
| | | 14q | TRAF3 |
| | | 16q | WWOX, CYLD |
| | | 17p | TP53 |
| Hyperdiploidy | | Gain | |
| Trisomies of chromosomes | | 1q | CKS1B, ANP32E |
| 3, 5, 7, 9, 11, 15, 19, 21 | | | |

The term “B cell proliferative disorder” or “B cell malignancy” refers to a disorder that is associated with some degree of abnormal B cell proliferation and includes, for example, a lymphoma, leukemia, myeloma, and myelodysplastic syndrome. In one embodiment, the B cell proliferative disorder is a lymphoma, such as non-Hodgkin's lymphoma (NHL), including, for example, diffuse large B cell lymphoma (DLBCL) (e.g., relapsed or refractory DLBCL). In another embodiment, the B cell proliferative disorder is a leukemia, such as chronic lymphocytic leukemia (CLL). Other specific examples of cancer also include germinal-center B cell-like (GCB) diffuse large B cell lymphoma (DLBCL), activated B cell-like (ABC) DLBCL, follicular lymphoma (FL), mantle cell lymphoma (MCL), acute myeloid leukemia (AML), chronic lymphoid leukemia (CLL), marginal zone lymphoma (MZL), small lymphocytic leukemia (SLL), lymphoplasmacytic lymphoma (LL), Waldenstrom macroglobulinemia (WM), central nervous system lymphoma (CNSL), Burkitt's lymphoma (BL), B cell prolymphocytic leukemia, splenic marginal zone lymphoma, hairy cell leukemia, splenic lymphoma/leukemia, unclassifiable, splenic diffuse red pulp small B cell lymphoma, hairy cell leukemia variant, heavy chain diseases, a heavy chain disease, γ heavy chain disease, p heavy chain disease, plasma cell myeloma, solitary plasmacytoma of bone, extraosseous plasmacytoma, extranodal marginal zone lymphoma of mucosa-associated lymphoid tissue (MALT lymphoma), nodal marginal zone lymphoma, pediatric nodal marginal zone lymphoma, pediatric follicular lymphoma, primary cutaneous follicle center lymphoma, T cell/histiocyte rich large B cell lymphoma, primary DLBCL of the CNS, primary cutaneous DLBCL, leg type, EBV-positive DLBCL of the elderly, DLBCL associated with chronic inflammation, lymphomatoid granulomatosis, primary mediastinal (thymic) large B cell lymphoma, intravascular large B cell lymphoma, ALK-positive large B cell lymphoma, plasmablastic lymphoma, large B cell lymphoma arising in HHV8-associated multicentric Castleman disease, primary effusion lymphoma: B cell lymphoma, unclassifiable, with features intermediate between DLBCL and Burkitt lymphoma, and B cell lymphoma, unclassifiable, with features intermediate between DLBCL and classical Hodgkin's lymphoma. Further examples of cancer include, but are not limited to, carcinoma, lymphoma, blastoma, sarcoma, and leukemia or lymphoid malignancies, including B cell lymphomas. More particular examples of such cancers include, but are not limited to, low grade/follicular NHL; small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; AIDS-related lymphoma; and acute lymphoblastic leukemia (ALL); chronic myeloblastic leukemia; and post-transplant lymphoproliferative disorder (PTLD). Examples of solid tumors include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer and gastrointestinal stromal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, superficial spreading melanoma, lentigo maligna melanoma, acral lentiginous melanomas, nodular melanomas, as well as abnormal vascular proliferation associated with phakomatoses, edema (such as that associated with brain tumors), Meigs' syndrome, brain, as well as head and neck cancer, and associated metastases. In certain embodiments, cancers that are amenable to treatment by the antibodies of the invention include breast cancer, colorectal cancer, rectal cancer, non-small cell lung cancer, glioblastoma, non-Hodgkins lymphoma (NHL), renal cell cancer, prostate cancer, liver cancer, pancreatic cancer, soft-tissue sarcoma, Kaposi's sarcoma, carcinoid carcinoma, head and neck cancer, ovarian cancer, and mesothelioma.

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) that are bound to their cognate antigen. To assess complement activation, a CDC assay, e.g., as described in Gazzano-Santoro et al., J. Immunol. Methods 202:163 (1996), can be performed.

“Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxic agents. The antibodies “arm” the cytotoxic cells and are absolutely required for such killing. The primary cells for mediating ADCC, NK cells, express FcγRIII only, whereas monocytes express FcγRI, FcγRII, and FcγRIII. FcR expression on hematopoietic cells is summarized in Table 3 on page 464 of Ravetch and Kinet. Annu. Rev. Immunol. 9:457-92, 1991. To assess ADCC activity of a molecule of interest, an in vitro ADCC assay, such as that described in U.S. Pat. No. 5,500,362 or 5,821,337 can be performed. Useful effector cells for such assays include peripheral blood mononuclear cells (PBMC) and Natural Killer (NK) cells. Alternatively, or additionally, ADCC activity of the molecule of interest can be assessed in vivo, e.g., in an animal model such as that disclosed in Clynes et al., Proc. Natl. Acad. Sci. USA. 95:652-656, 1998.

“Complex” or “complexed” as used herein refers to the association of two or more molecules that interact with each other through bonds and/or forces (e.g., Van der Waals, hydrophobic, hydrophilic forces) that are not peptide bonds. In one aspect, the complex is heteromultimeric. It should be understood that the term “protein complex” or “polypeptide complex” as used herein includes complexes that have a non-protein entity conjugated to a protein in the protein complex (e.g., including, but not limited to, chemical molecules such as a toxin or a detection agent).

As used herein, “delaying progression” of a disorder or disease means to defer, hinder, slow, retard, stabilize, and/or postpone development of the disease or disorder (e.g., a cell proliferative disorder, e.g., cancer (e.g., MM)). This delay can be of varying lengths of time, depending on the history of the disease and/or individual being treated. As is evident to one skilled in the art, a sufficient or significant delay can, in effect, encompass prevention, in that the individual does not develop the disease. For example, a late-stage cancer, such as development of metastasis, may be delayed.

An “effective amount” of a compound, for example, an anti-FcRH5/anti-CD3 T-cell-dependent bispecific antibody (TDB) of the invention or a composition (e.g., pharmaceutical composition) thereof, is at least the minimum amount required to achieve the desired therapeutic or prophylactic result, such as a measurable improvement or prevention of a particular disorder (e.g., a cell proliferative disorder, e.g., cancer). An effective amount herein may vary according to factors such as the disease state, age, sex, and weight of the patient, and the ability of the antibody to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications, and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, “overall survival” or “OS” refers to the percentage of individuals in a group who are likely to be alive after a particular duration of time.

As used herein, “objective response rate” (ORR) refers to the sum of stringent complete response (sCR), complete response (CR), very good partial response (VGPR), and partial response (PR) rates as determined using the International Myeloma Working Group response criteria (e.g., see Table 6A and 6B in Example 1).

The term “epitope” refers to the particular site on an antigen molecule to which an antibody binds. In some aspects, the particular site on an antigen molecule to which an antibody binds is determined by hydroxyl radical footprinting. In some aspects, the particular site on an antigen molecule to which an antibody binds is determined by crystallography.

A “growth inhibitory agent” when used herein refers to a compound or composition which inhibits growth of a cell either in vitro or in vivo. In one aspect, growth inhibitory agent is growth inhibitory antibody that prevents or reduces proliferation of a cell expressing an antigen to which the antibody binds. In another aspect, the growth inhibitory agent may be one which significantly reduces the percentage of cells in S phase. Aspects of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in Mendelsohn and Israel, eds., The Molecular Basis of Cancer, Chapter 1, entitled “Cell cycle regulation, oncogenes, and antineoplastic drugs” by Murakami et al. (W. B. Saunders, Philadelphia, 1995), e.g., p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

An “immunoconjugate” is an antibody conjugated to one or more heterologous molecule(s), including but not limited to a cytotoxic agent.

The term “immunomodulatory agent” or “IMiD” refers to a class of molecules that modifies the immune system response or the functioning of the immune system. Immunomodulatory agents include, but are not limited to, POMALYST® (pomalidomide), thalidomide (α-N-phthalimido-glutarimide) and its analogues, OTEZLA® (apremilast), REVLIMID® (lenalidomide) and PD-1 axis binding antagonists and pharmaceutically acceptable salts or acids thereof.

A “subject” or an “individual” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain aspects, the subject or individual is a human. The subject may be a patient. In some instances, the subject is an adult.

An “isolated” protein or peptide is one which has been separated from a component of its natural environment. In some aspects, a protein or peptide is purified to greater than 95% or 99% purity as determined by, for example, electrophoresis (e.g., sodium dodecyl sulfate-polyacrylamide gel electrophoresis (SDS-PAGE), isoelectric focusing (IEF), capillary electrophoresis) or chromatography (e.g., ion exchange or reverse phase HPLC).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “PD-1 axis binding antagonist” refers to a molecule that inhibits the interaction of a PD-1 axis binding partner with either one or more of its binding partners, so as to remove T-cell dysfunction resulting from signaling on the PD-1 signaling axis, with a result being to restore or enhance T-cell function (e.g., proliferation, cytokine production, and/or target cell killing). As used herein, a PD-1 axis binding antagonist includes a PD-L1 binding antagonist, a PD-1 binding antagonist, and a PD-L2 binding antagonist. In some instances, the PD-1 axis binding antagonist includes a PD-L1 binding antagonist or a PD-1 binding antagonist. In a preferred aspect, the PD-1 axis binding antagonist is a PD-L1 binding antagonist.

The term “PD-L1 binding antagonist” refers to a molecule that decreases, blocks, inhibits, abrogates, or interferes with signal transduction resulting from the interaction of PD-L1 with either one or more of its binding partners, such as PD-1 and/or B7-1. In some instances, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some instances, the PD-L1 binding antagonists include anti-PD-L1 antibodies, antigen-binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1 and/or B7-1. In one instance, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some instances, the PD-L1 binding antagonist binds to PD-L1. In some instances, a PD-L1 binding antagonist is an anti-PD-L1 antibody (e.g., an anti-PD-L1 antagonist antibody). Exemplary anti-PD-L1 antagonist antibodies include atezolizumab, MDX-1105, MEDI4736 (durvalumab), MSB0010718C (avelumab), SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, KD036, KY1003, YBL-007, and HS-636. In some aspects, the anti-PD-L1 antibody is atezolizumab, MDX-1105, MEDI4736 (durvalumab), or MSB0010718C (avelumab). In one specific aspect, the PD-L1 binding antagonist is MDX-1105. In another specific aspect, the PD-L1 binding antagonist is MEDI4736 (durvalumab). In another specific aspect, the PD-L1 binding antagonist is MSB0010718C (avelumab). In other aspects, the PD-L1 binding antagonist may be a small molecule, e.g., GS-4224, INCB086550, MAX-10181, INCB090244, CA-170, or ABSK041, which in some instances may be administered orally. Other exemplary PD-L1 binding antagonists include AVA-004, MT-6035, VXM10, LYN192, GB7003, and JS-003. In a preferred aspect, the PD-L1 binding antagonist is atezolizumab. Atezolizumab is also described in WHO Drug Information (International Nonproprietary Names for Pharmaceutical Substances), Proposed INN: List 112, Vol. 28, No. 4, published Jan. 16, 2015 (see page 485).

The term “PD-1 binding antagonist” refers to a molecule that decreases, blocks, inhibits, abrogates or interferes with signal transduction resulting from the interaction of PD-1 with one or more of its binding partners, such as PD-L1 and/or PD-L2. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. In some instances, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen-binding fragments thereof, immunoadhesins, fusion proteins, oligopeptides, and other molecules that decrease, block, inhibit, abrogate, or interfere with signal transduction resulting from the interaction of PD-1 with PD-L1 and/or PD-L2. In one instance, a PD-1 binding antagonist reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some instances, the PD-1 binding antagonist binds to PD-1. In some instances, the PD-1 binding antagonist is an anti-PD-1 antibody (e.g., an anti-PD-1 antagonist antibody). Exemplary anti-PD-1 antagonist antibodies include nivolumab, pembrolizumab, MEDI-0680, PDR001 (spartalizumab), REGN2810 (cemiplimab), BGB-108, prolgolimab, camrelizumab, sintilimab, tislelizumab, toripalimab, dostarlimab, retifanlimab, sasanlimab, penpulimab, CS1003, HLX10, SCT-I10A, zimberelimab, balstilimab, genolimzumab, BI 754091, cetrelimab, YBL-006, BAT1306, HX008, budigalimab, AMG 404, CX-188, JTX-4014, 609A, Sym021, LZM009, F520, SG001, AM0001, ENUM 244C8, ENUM 388D4, STI-1110, AK-103, and hAb21. In a specific aspect, a PD-1 binding antagonist is MDX-1106 (nivolumab). In another specific aspect, a PD-1 binding antagonist is MK-3475 (pembrolizumab). In another specific aspect, a PD-1 binding antagonist is a PD-L2 Fc fusion protein, e.g., AMP-224. In another specific aspect, a PD-1 binding antagonist is MED1-0680. In another specific aspect, a PD-1 binding antagonist is PDR001 (spartalizumab). In another specific aspect, a PD-1 binding antagonist is REGN2810 (cemiplimab). In another specific aspect, a PD-1 binding antagonist is BGB-108. In another specific aspect, a PD-1 binding antagonist is prolgolimab. In another specific aspect, a PD-1 binding antagonist is camrelizumab. In another specific aspect, a PD-1 binding antagonist is sintilimab. In another specific aspect, a PD-1 binding antagonist is tislelizumab. In another specific aspect, a PD-1 binding antagonist is toripalimab. Other additional exemplary PD-1 binding antagonists include BION-004, CB201, AUNP-012, ADG104, and LBL-006.

The term “PD-L2 binding antagonist” refers to a molecule that decreases, blocks, inhibits, abrogates or interferes with signal transduction resulting from the interaction of PD-L2 with either one or more of its binding partners, such as PD-1. PD-L2 (programmed death ligand 2) is also referred to in the art as “programmed cell death 1 ligand 2,” “PDCD1LG2,” “CD273,” “B7-DC,” “Btdc,” and “PDL2.” An exemplary human PD-L2 is shown in UniProtKB/Swiss-Prot Accession No. Q9BQ51. In some instances, a PD-L2 binding antagonist is a molecule that inhibits the binding of PD-L2 to one or more of its binding partners. In a specific aspect, the PD-L2 binding antagonist inhibits binding of PD-L2 to PD-1. Exemplary PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one aspect, a PD-L2 binding antagonist reduces the negative co-stimulatory signal mediated by or through cell surface proteins expressed on T lymphocytes mediated signaling through PD-L2 so as render a dysfunctional T-cell less dysfunctional (e.g., enhancing effector responses to antigen recognition). In some aspects, the PD-L2 binding antagonist binds to PD-L2. In some aspects, a PD-L2 binding antagonist is an immunoadhesin. In other aspects, a PD-L2 binding antagonist is an anti-PD-L2 antagonist antibody.

The term “protein,” as used herein, refers to any native protein from any vertebrate source, including mammals such as primates (e.g., humans) and rodents (e.g., mice and rats), unless otherwise indicated. The term encompasses “full-length,” unprocessed protein as well as any form of the protein that results from processing in the cell. The term also encompasses naturally occurring variants of the protein, e.g., splice variants or allelic variants.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity for the purposes of the alignment. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. Alternatively, the percent identity values can be generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087 and is described in WO 2001/007611.

Unless otherwise indicated, for purposes herein, percent amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448; W. R. Pearson (1996) “Effective protein sequence comparison” Meth. Enzymol. 266:227-258; and Pearson et. al. (1997) Genomics 46:24-36 and is publicly available from www.fasta.bioch.virginia.edu/fasta_www2/fasta_down.shtml or www.ebi.ac.uk/Tools/sss/fasta. Alternatively, a public server accessible at fasta.bioch.virginia.edu/fasta_www2/index.cgi can be used to compare the sequences, using the ggsearch (global protein:protein) program and default options (BLOSUM50; open: −10; ext: −2; Ktup=2) to ensure a global, rather than local, alignment is performed. Percent amino acid identity is given in the output alignment header.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one-time administration and typical dosages range from 10 to 200 units (Grays) per day.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some aspects, antibodies of the present disclosure (e.g., anti-FcRH5/anti-CD3 TDBs of the present disclosure) are used to delay development of a disease or to slow the progression of a disease.

By “reduce” or “inhibit” is meant the ability to cause an overall decrease, for example, of 20% or greater, of 50% or greater, or of 75%, 85%, 90%, 95%, or greater. In certain aspects, reduce or inhibit can refer to the effector function of an antibody that is mediated by the antibody Fc region, such effector functions specifically including CDC, ADCC, and ADCP.

According to the invention, the term “vaccine” relates to a pharmaceutical preparation (pharmaceutical composition) or product that upon administration induces an immune response, in particular a cellular immune response, which recognizes and attacks a pathogen or a diseased cell such as a cancer cell. A vaccine may be used for the prevention or treatment of a disease. A vaccine may be a cancer vaccine. A “cancer vaccine” as used herein is a composition that stimulates an immune response in a subject against a cancer. Cancer vaccines typically consist of a source of cancer-associated material or cells (antigen) that may be autologous (from self) or allogenic (from others) to the subject, along with other components (e.g., adjuvants) to further stimulate and boost the immune response against the antigen. Cancer vaccines can result in stimulating the immune system of the subject to produce antibodies to one or several specific antigens, and/or to produce killer T cells to attack cancer cells that have those antigens.

As used herein, “administering” is meant a method of giving a dosage of a compound (e.g., an anti-FcRH5/anti-CD3 TDB such as cevostamab, an IMiD (e.g., pomalidomide), an anti-CD38 antibody (e.g., daratumumab), or a corticosteroid (e.g., dexamethasone)) to a subject. In some aspects, the compositions utilized in the methods herein are administered intravenously. The compositions utilized in the methods described herein can be administered, for example, intramuscularly, intravenously, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. The method of administration can vary depending on various factors (e.g., the compound or composition being administered and the severity of the condition, disease, or disorder being treated).

“CD38” as used herein refers to a glycoprotein found on the surface of many immune cells, including CD4+, CD8+, B lymphocytes, and natural killer (NK) cells, and includes any native CD38 from any vertebrate source, including mammals such as primates (e.g., humans) and rodents (e.g., mice and rats), unless otherwise indicated. CD38 is typically expressed at a higher level and more uniformly on myeloma cells as compared to normal lymphoid and myeloid cells. The term encompasses “full-length,” unprocessed CD38, as well as any form of CD38 that results from processing in the cell. The term also encompasses naturally occurring variants of CD38, e.g., splice variants or allelic variants. CD38 is also referred to in the art as cluster of differentiation 38, ADP-ribosyl cyclase 1, cADPr hydrolase 1, and cyclic ADP-ribose hydrolase 1. CD38 is encoded by the CD38 gene. The nucleic acid sequence of an exemplary human CD38 is shown under NCBI Reference Sequence: NM_001775.4 or in SEQ ID NO: 33. The amino acid sequence of an exemplary human CD38 protein encoded by CD38 is shown under UniProt Accession No. P28907 or in SEQ ID NO: 34.

The term “anti-CD38 antibody” encompasses all antibodies that bind CD38 with sufficient affinity such that the antibody is useful as a therapeutic agent in targeting a cell expressing the antigen, and does not significantly cross-react with other proteins such as a negative control protein in the assays described below. For example, an anti-CD38 antibody may bind to CD38 on the surface of a MM cell and mediate cell lysis through the activation of complement-dependent cytotoxicity, ADCC, antibody-dependent cellular phagocytosis (ADCP), and apoptosis mediated by Fc cross-linking, leading to the depletion of malignant cells and reduction of the overall cancer burden. An anti-CD38 antibody may also modulate CD38 enzyme activity through inhibition of ribosyl cyclase enzyme activity and stimulation of the cyclic adenosine diphosphate ribose (cADPR) hydrolase activity of CD38. In certain aspects, an anti-CD38 antibody that binds to CD38 has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). In certain aspects, the anti-CD38 antibody may bind to both human CD38 and chimpanzee CD38. Anti-CD38 antibodies also include anti-CD38 antagonist antibodies. Bispecific antibodies wherein one arm of the antibody binds CD38 are also contemplated. Also encompassed by this definition of anti-CD38 antibody are functional fragments of the preceding antibodies. Examples of antibodies which bind CD38 include: daratumumab (DARZALEX®) (U.S. Pat. No. 7,829,673 and U.S. Pub. No: 20160067205 A1); “MOR202” (U.S. Pat. No. 8,263,746); and isatuximab (SAR-650984).

As used herein, “triple-class refractory” refers to a patient (e.g., a MM patient) that has been previously exposed to and is refractory to at least one proteosome inhibitor (PI; e.g., bortezomib, carfilzomib, or ixazomib), at least one immunomodulatory drug (IMiD; e.g. thalidomide, lenalidomide, or pomalidomide), and at least one anti-CD38 antibody (e.g., daratumumab, MOR202, or isatuximab).

### II. Therapeutic Methods

The invention is based, in part, on methods of treating a subject having cancer (e.g., multiple myeloma (MM)) using dosing regimens, including fractionated, dose-escalation dosing regimens with anti-fragment crystallizable receptor-like 5 (FcRH5)/anti-cluster of differentiation 3 (CD3) bispecific antibodies. The dosing regimen described herein may be used for subjects having a triple-class refractory MM and who have previously received a B cell maturation factor (BCMA)-targeting therapy, such as a T-cell dependent bispecific (TDB) antibody. An exemplary dosing regimen described herein is of cevostamab administered on Day 1, Day 2, and Day 8 for a first dosing cycle (C1) and Q3W for each subsequent cycle. For example, the subject may be administered 0.3 mg of cevostamab on Day 1, 3.3 mg of cevostamab on Day 2, and a target dose (e.g., 160 mg) of cevostamab on Day 8 for the C1. This split 0.3/3.3 mg dosing regimen and/or dosing on Day 1 and Day 2 are expected to reduce or inhibit unwanted treatment effects when delivering a target dose (e.g., 160 mg), which include cytokine-driven toxicities (e.g., cytokine release syndrome (CRS)), infusion-related reactions (IRRs), macrophage activation syndrome (MAS), neurologic toxicities, severe tumor lysis syndrome (TLS), neutropenia, thrombocytopenia, and/or elevated liver enzymes. Furthermore, administration of the split 0.3/3.3 mg dosing regimen and/or dosing on Day 1 and Day 2 allows for an earlier therapeutic delivery that is more tolerable in rapidly progressing late line R/R MM subjects. Therefore, the methods are useful for treating the subject while achieving a more favorable benefit-risk profile.

In the event of an unwanted treatment effect (e.g., CRS, IRR, MAS, or TLS), for example, the dosing regimen described herein may include the administration of cevostamab on Day 1, Day 3, and Day 8 for a first dosing cycle (C1) and Q3W for each subsequent cycle. For example, the subject may be administered 0.3 mg of cevostamab on Day 1, 3.3 mg of cevostamab on Day 3, and a target dose (e.g., 160 mg) of cevostamab on Day 8 for the C1. In another example, the dosing regimen may include the administration of cevostamab on Day 1, Day 4, and Day 8 for a first dosing cycle (C1) and Q3W for each subsequent cycle. For example, the subject may be administered 0.3 mg of cevostamab on Day 1, 3.3 mg of cevostamab on Day 4, and a target dose (e.g., 160 mg) of cevostamab on Day 8 for the C1.

### A. Dosing Regimens

i. Single Step-Up Dosing Regimens

In some aspects, the invention provides methods of treating a subject having a cancer (e.g., an MM) comprising administering to the subject a bispecific antibody that binds to FcRH5 and CD3 in a single step-up dosing regimen. In some examples, the subject has a triple-class refractory MM and has previously received a BCMA-targeting therapeutic agent.

In some aspects, the invention provides a method of treating a subject having an MM comprising administering to the subject a bispecific antibody that binds to FcRH5 and CD3 in a dosing regimen comprising at least a first dosing cycle, wherein the first dosing cycle comprises a first dose (C1D1) of the bispecific antibody and a second dose (C1D2) of the bispecific antibody, wherein the C1D1 is between about 0.05 mg to about 180 mg (e.g., between about 0.1 mg to about 160 mg, between about 0.5 mg to about 140 mg, between about 1 mg to about 120 mg, between about 1.5 mg to about 100 mg, between about 2.0 mg to about 80 mg, between about 2.5 mg to about 50 mg, between about 3.0 mg to about 25 mg, between about 3.0 mg to about 15 mg, between about 3.0 mg to about 10 mg, or between about 3.0 mg to about 5 mg) and the C1D2 is between about 0.15 mg to about 1000 mg (e.g., between about 0.5 mg to about 800 mg, between about 1 mg to about 700 mg, between about 5 mg to about 500 mg, between about 10 mg to about 400 mg, between about 25 mg to about 300 mg, between about 40 mg to about 200 mg, between about 50 mg to about 100 mg, between about 75 mg to about 100 mg, or between about 85 mg to about 100 mg). In some examples, the C1D1 is administered on Day 1 of the dosing cycle and the C1D2 is administered on Day 2 of the dosing cycle. In another example, the C1D1 is administered on Day 1 of the dosing cycle and the C1D2 is administered on Day 3 of the dosing cycle. In yet another example, the C1D1 is administered on Day 1 of the dosing cycle and the C1D2 is administered on Day 4 of the dosing cycle. The dosing cycle may have any suitable duration, e.g., 7 days, 14 days, 21 days, 28 days, or longer.

In some aspects, the invention provides a method of treating a subject having a cancer (e.g., an MM) comprising administering to the subject a bispecific antibody that binds to FcRH5 and CD3 in a dosing regimen comprising at least a first dosing cycle and a second dosing cycle, wherein (a) the first dosing cycle comprises a first dose (C1D1; cycle 1, dose 1) of the bispecific antibody and a second dose (C1D2; cycle 1, dose, 2) of the bispecific antibody, wherein the C1D1 is less than the C1D2, and wherein the C1D1 is between about 0.05 mg to about 180 mg (e.g., between about 0.1 mg to about 160 mg, between about 0.5 mg to about 140 mg, between about 1 mg to about 120 mg, between about 1.5 mg to about 100 mg, between about 2.0 mg to about 80 mg, between about 2.5 mg to about 50 mg, between about 3.0 mg to about 25 mg, between about 3.0 mg to about 15 mg, between about 3.0 mg to about 10 mg, or between about 3.0 mg to about 5 mg) and the C1D2 is between about 0.15 mg to about 1000 mg (e.g., between about 0.5 mg to about 800 mg, between about 1 mg to about 700 mg, between about 5 mg to about 500 mg, between about 10 mg to about 400 mg, between about 25 mg to about 300 mg, between about 40 mg to about 200 mg, between about 50 mg to about 100 mg, between about 75 mg to about 100 mg, or between about 85 mg to about 100 mg); and (b) the second dosing cycle comprises a single dose (C2D1; cycle 2, dose 1) of the bispecific antibody, wherein the C2D1 is equal to or greater than the C1D2 and is between about 0.15 mg to about 1000 mg (e.g., between about 0.5 mg to about 800 mg, between about 1 mg to about 700 mg, between about 5 mg to about 500 mg, between about 10 mg to about 400 mg, between about 25 mg to about 300 mg, between about 40 mg to about 200 mg, between about 50 mg to about 100 mg, between about 75 mg to about 100 mg, or between about 85 mg to about 100 mg). In some examples, the C1D1 is administered on Day 1 of the dosing cycle and the C1D2 is administered on Day 2 of the dosing cycle. In another example, the C1D1 is administered on Day 1 of the dosing cycle and the C1D2 is administered on Day 3 of the dosing cycle. In yet another example, the C1D1 is administered on Day 1 of the dosing cycle and the C1D2 is administered on Day 4 of the dosing cycle. The dosing cycle may have any suitable duration, e.g., 7 days, 14 days, 21 days, 28 days, or longer.

In some aspects, (a) the C1D1 is between about 0.5 mg to about 19.9 mg (e.g., between about 1 mg to about 18 mg, between about 2 mg to about 15 mg, between about 3 mg to about 10 mg, between about 3.3 mg to about 6 mg, or between about 3.4 mg to about 4 mg, e.g., about 3 mg, 3.1 mg, 3.2 mg, 3.3 mg, 3.4 mg, 3.5 mg, 3.6 mg, 3.7 mg, 3.8 mg, 3.8 mg, 4 mg, 4.1 mg, 4.2 mg, 4.3 mg, 4.4 mg, 4.5 mg, 4.6 mg, 4.7 mg, 4.8 mg, 4.9 mg, 5 mg, 5.2 mg, 5.6 mg, 5.8 mg, 6 mg, 6.2 mg, 6.4 mg, 6.6 mg, 6.8 mg, 7 mg, 7.2 mg, 7.4 mg, 7.6 mg, 7.8 mg, 8 mg, 8.2 mg, 8.4 mg, 8.6 mg, 8.8 mg, 9 mg, 9.2 mg, 9.4 mg, 9.6 mg, 9.8 mg, 10 mg, 10.2 mg, 10.4 mg, 10.6 mg, 10.8 mg, 11 mg, 11.2 mg, 11.4 mg, 11.6 mg, 11.8 mg, 12 mg, 12.2 mg, 12.4 mg, 12.6 mg, 12.8 mg, 13 mg, 13.2 mg, 13.4 mg, 13.6 mg, 13.8 mg, 14 mg, 14.2 mg, 14.4 mg, 14.6 mg, 14.8 mg, 15 mg, 15.2 mg, 15.4 mg, 15.6 mg, 15.8 mg, 16 mg, 16.2 mg, 16.4 mg, 16.6 mg, 16.8 mg, 17 mg, 18.2 mg, 18.4 mg, 18.6 mg, 18.8 mg, 19 mg, 19.2 mg, 19.4 mg, 19.6 mg, or 19.8 mg), and (b) the C1D2 is between about 20 mg to about 600 mg (e.g., between about 30 mg to 500 mg, 40 mg to 400 mg, 60 mg to 350 mg, 80 mg to 300 mg, 100 mg to 200 mg, or 140 mg to 180 mg, e.g., about 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, or 600 mg).

In some aspects, the C1D1 is between about 1.2 mg to about 10.8 mg and the C1D2 is between about 80 mg to about 300 mg. In some aspects, the C1D1 is between 1.2 mg to 10.8 mg and the C1D2 is between 80 mg to 300 mg. In some aspects, the C1D1 is 3.3 mg and the C1D2 is 40 mg. In some aspects, the C1D1 is 3.3 mg and the C1D2 is 90 mg. In some aspects, the C1D1 is 3.3 mg and the C1D2 is 120 mg. In some aspects, the C1D1 is 3.3 mg and the C1D2 is 132 mg. In some aspects, the C1D1 is 3.3 mg and the C1D2 is 160 mg. In some aspects, the C1D1 is 3.3 mg and the C1D2 is 198 mg. In some aspects, the C1D1 is 3.3 mg and the C1D2 is 252 mg. In some aspects, the C1D1 is 3.6 mg and the C1D2 is 40 mg. In some aspects, the C1D1 is 3.6 mg and the C1D2 is 90 mg. In some aspects, the C1D1 is 3.6 mg and the C1D2 is 120 mg. In some aspects, the C1D1 is 3.6 mg and the C1D2 is 132 mg. In some aspects, the C1D1 is 3.6 mg and the C1D2 is 160 mg. In some aspects, the C1D1 is 3.6 mg and the C1D2 is 198 mg. In some aspects, the C1D1 is 3.6 mg and the C1D2 is 252 mg.

In some instances, the methods described above may include a first dosing cycle of two weeks or 14 days. In some instances, the methods described above may include a first dosing cycle of three weeks or 21 days. In some instances, the methods described above may include a first dosing cycle of four weeks or 28 days.

In some instances, the methods described above may include a first dosing cycle of three weeks or 21 days. In some instances, the methods may include administering to the subject the C1D1 and the C1D2 on or about Days 1 and 2, respectively, of the first dosing cycle.

In some instances, the methods described above may include a first dosing cycle of three weeks or 21 days. In some instances, the methods may include administering to the subject the C1D1 and the C1D2 on or about Days 1 and 3, respectively, of the first dosing cycle (e.g., in the event of an unwanted treatment effect, such as a CRS, IRR, MAS, or TLS). In other instances, the methods may include administering to the subject the C1D1 and the C1D2 on or about Days 1 and 4, respectively, of the first dosing cycle (e.g., in the event of an unwanted treatment effect, such as a CRS, IRR, MAS, or TLS).

ii. Double Step-Up Dosing Regimens

In other aspects, the invention provides methods of treating a subject having a cancer (e.g., an MM) comprising administering to the subject a bispecific antibody that binds to FcRH5 and CD3 in a double step-up dosing regimen. In some examples, the subject has a triple-class refractory MM and has previously received a BCMA-targeting therapeutic agent.

In some aspects, the disclosure features a method of treating a subject having a cancer (e.g., an MM) comprising administering to the subject a bispecific antibody that binds to FcRH5 and CD3 in a dosing regimen comprising at least a first dosing cycle, wherein the first dosing cycle comprises a first dose (C1D1) of the bispecific antibody, a second dose (C1D2) of the bispecific antibody, and a third dose (C1D3) of the bispecific antibody, wherein the C1D1 is between about 0.2 mg to about 0.4 mg (e.g., is about 0.20 mg, 0.21 mg, 0.22 mg, 0.23 mg, 0.24 mg, 0.25 mg, 0.26 mg, 0.27 mg, 0.28 mg, 0.29 mg, 0.30 mg, 0.31 mg, 0.32 mg, 0.33 mg, 0.34 mg, 0.35 mg, 0.36 mg, 0.37 mg, 0.38 mg, 0.39 mg, or 0.40 mg); the C1D2 is greater than the C1D1, and the C1D3 is greater than the C1D2. In some aspects, the C1D1 is about 0.3 mg. In some examples, the C1D1 is administered on Day 1 of the dosing cycle and the C1D2 is administered on Day 2 of the dosing cycle. In some examples, the C1D1 is administered on Day 1 of the dosing cycle and the C1D2 is administered on Day 3 of the dosing cycle. In some examples, the C1D1 is administered on Day 1 of the dosing cycle and the C1D2 is administered on Day 4 of the dosing cycle. In some examples, the C1D1 is administered on Day 1 of the dosing cycle and the C1D2 is administered on Day 5 of the dosing cycle. In some examples, the C1D1 is administered on Day 1 of the dosing cycle and the C1D2 is administered on Day 6 of the dosing cycle. In some examples, the C1D1 is administered on Day 1 of the dosing cycle and the C1D2 is administered on Day 7 of the dosing cycle. The dosing cycle may have any suitable duration, e.g., 7 days, 14 days, 21 days, 28 days, or longer.

In some aspects, the C1D1 is between 0.2 mg to and 0.4 mg (e.g., is 0.20 mg, 0.21 mg, 0.22 mg, 0.23 mg, 0.24 mg, 0.25 mg, 0.26 mg, 0.27 mg, 0.28 mg, 0.29 mg, 0.30 mg, 0.31 mg, 0.32 mg, 0.33 mg, 0.34 mg, 0.35 mg, 0.36 mg, 0.37 mg, 0.38 mg, 0.39 mg, or 0.40 mg). In some aspects, the C1D1 is 0.3 mg.

In some aspects, the disclosure provides a method of treating a subject having a cancer (e.g., an MM) comprising administering to the subject a bispecific antibody that binds to FcRH5 and CD3 in a dosing regimen comprising at least a first dosing cycle, wherein the first dosing cycle comprises a first dose (C1D1) of the bispecific antibody, a second dose (C1D2) of the bispecific antibody, and a third dose (C1D3) of the bispecific antibody, wherein the C1D1 is between about 0.01 mg to about 2.9 mg, the C1D2 is between about 3 mg to about 19.9 mg, and the C1D3 is between about 20 mg to about 600 mg. In some examples, the C1D1 is administered on Day 1 of the dosing cycle and the C1D2 is administered on Day 2 of the dosing cycle. In some examples, the C1D1 is administered on Day 1 of the dosing cycle and the C1D2 is administered on Day 3 of the dosing cycle. In some examples, the C1D1 is administered on Day 1 of the dosing cycle and the C1D2 is administered on Day 4 of the dosing cycle. The dosing cycle may have any suitable duration, e.g., 7 days, 14 days, 21 days, 28 days, or longer.

In some aspects, the invention provides a method of treating a subject having a cancer (e.g., an MM) comprising administering to the subject a bispecific antibody that binds to FcRH5 and CD3 in a dosing regimen comprising at least a first dosing cycle and a second dosing cycle, wherein (a) the first dosing cycle comprises a first dose (C1D1) of the bispecific antibody, a second dose (C1D2) of the bispecific antibody, and a third dose (C1D3) of the bispecific antibody, wherein the C1D1 and the C1D2 are each less than the C1D3, and wherein the C1D1 is between about 0.01 mg to about 2.9 mg, the C1D2 is between about 3 mg to about 19.9 mg, and the C1D3 is between about 20 mg to about 600 mg; and (b) the second dosing cycle comprises a single dose (C2D1) of the bispecific antibody, wherein the C2D1 is equal to or greater than the C1D3 and is between about 20 mg to about 600 mg. In some examples, the C1D1 is administered on Day 1 of the dosing cycle and the C1D2 is administered on Day 2 of the dosing cycle. In some examples, the C1D1 is administered on Day 1 of the dosing cycle and the C1D2 is administered on Day 3 of the dosing cycle. In some examples, the C1D1 is administered on Day 1 of the dosing cycle and the C1D2 is administered on Day 4 of the dosing cycle. The dosing cycle may have any suitable duration, e.g., 7 days, 14 days, 21 days, 28 days, or longer.

In some aspects, the C1D1 is between about 0.05 mg to about 2.5 mg, about 0.1 mg to about 2 mg, about 0.2 mg to about 1 mg, or about 0.2 mg to about 0.4 mg (e.g., about 0.01 mg, 0.05 mg, 0.1 mg, 0.2 mg, 0.3 mg, 0.4 mg, 0.5 mg, 0.6 mg, 0.7 mg, 0.9 mg, 1 mg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, or 2.9 mg). In some aspects, the C1D1 is about 0.3 mg.

In some aspects, the C1D1 is between 0.05 mg to 2.5 mg, 0.1 mg to 2 mg, 0.2 mg to 1 mg, or 0.2 mg to 0.4 mg (e.g., 0.01 mg, 0.05 mg, 0.1 mg, 0.2 mg, 0.3 mg, 0.4 mg, 0.5 mg, 0.6 mg, 0.7 mg, 0.9 mg, 1 mg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, or 2.9 mg). In some aspects, the C1D1 is 0.3 mg.

In some aspects, the C1D2 is between about 3 mg to about 19.9 mg (e.g., between about 3 mg to about 18 mg, between about 3.1 mg to about 15 mg, between about 3.2 mg to about 10 mg, between about 3.3 mg to about 6 mg, or between about 3.4 mg to about 4 mg, e.g., about 3 mg, 3.1 mg, 3.2 mg, 3.3 mg, 3.4 mg, 3.5 mg, 3.6 mg, 3.7 mg, 3.8 mg, 3.9 mg, 4 mg, 4.1 mg, 4.2 mg, 4.3 mg, 4.4 mg, 4.5 mg, 4.6 mg, 4.7 mg, 4.8 mg, 4.9 mg, 5 mg, 5.2 mg, 5.6 mg, 5.8 mg, 6 mg, 6.2 mg, 6.4 mg, 6.6 mg, 6.8 mg, 7 mg, 7.2 mg, 7.4 mg, 7.6 mg, 7.8 mg, 8 mg, 8.2 mg, 8.4 mg, 8.6 mg, 8.8 mg, 9 mg, 9.2 mg, 9.4 mg, 9.6 mg, 9.8 mg, 10 mg, 10.2 mg, 10.4 mg, 10.6 mg, 10.8 mg, 11 mg, 11.2 mg, 11.4 mg, 11.6 mg, 11.8 mg, 12 mg, 12.2 mg, 12.4 mg, 12.6 mg, 12.8 mg, 13 mg, 13.2 mg, 13.4 mg, 13.6 mg, 13.8 mg, 14 mg, 14.2 mg, 14.4 mg, 14.6 mg, 14.8 mg, 15 mg, 15.2 mg, 15.4 mg, 15.6 mg, 15.8 mg, 16 mg, 16.2 mg, 16.4 mg, 16.6 mg, 16.8 mg, 17 mg, 18.2 mg, 18.4 mg, 18.6 mg, 18.8 mg, 19 mg, 19.2 mg, 19.4 mg, 19.6 mg, or 19.8 mg). In some aspects, the C1D2 is between about 3.2 mg to about 10 mg. In some aspects, the C1D2 is about 3.6 mg. In some aspects, the C1D2 is about 3.3 mg.

In some aspects, the C1D2 is between 3 mg to 19.9 mg (e.g., between 3 mg to 18 mg, between 3.1 mg to 15 mg, between 3.2 mg to 10 mg, between 3.3 mg to 6 mg, or between 3.4 mg to 4 mg, e.g., 3 mg, 3.1 mg, 3.2 mg, 3.3 mg, 3.4 mg, 3.5 mg, 3.6 mg, 3.7 mg, 3.8 mg, 3.9 mg, 4 mg, 4.1 mg, 4.2 mg, 4.3 mg, 4.4 mg, 4.5 mg, 4.6 mg, 4.7 mg, 4.8 mg, 4.9 mg, 5 mg, 5.2 mg, 5.6 mg, 5.8 mg, 6 mg, 6.2 mg, 6.4 mg, 6.6 mg, 6.8 mg, 7 mg, 7.2 mg, 7.4 mg, 7.6 mg, 7.8 mg, 8 mg, 8.2 mg, 8.4 mg, 8.6 mg, 8.8 mg, 9 mg, 9.2 mg, 9.4 mg, 9.6 mg, 9.8 mg, 10 mg, 10.2 mg, 10.4 mg, 10.6 mg, 10.8 mg, 11 mg, 11.2 mg, 11.4 mg, 11.6 mg, 11.8 mg, 12 mg, 12.2 mg, 12.4 mg, 12.6 mg, 12.8 mg, 13 mg, 13.2 mg, 13.4 mg, 13.6 mg, 13.8 mg, 14 mg, 14.2 mg, 14.4 mg, 14.6 mg, 14.8 mg, 15 mg, 15.2 mg, 15.4 mg, 15.6 mg, 15.8 mg, 16 mg, 16.2 mg, 16.4 mg, 16.6 mg, 16.8 mg, 17 mg, 18.2 mg, 18.4 mg, 18.6 mg, 18.8 mg, 19 mg, 19.2 mg, 19.4 mg, 19.6 mg, or 19.8 mg). In some aspects, the C1D2 is between 3.2 mg to 10 mg. In some aspects, the C1D2 is 3.6 mg. In some aspects, the C1D2 is about 3.3 mg.

In some aspects, the C1D3 is between about 20 mg to about 600 mg (e.g., between about 30 mg to about 500 mg, about 40 mg to about 400 mg, about 60 mg to about 350 mg, about 80 mg to about 300 mg, about 100 mg to about 200 mg, or about 140 mg to about 180 mg, e.g., about 20, 40, 60, 80, 100, 120, 140, 160, 180, 200, 220, 240, 260, 280, 300, 320, 340, 360, 380, 400, 420, 440, 460, 480, 500, 520, 540, 560, 580, or 600 mg). In some aspects, the C1D3 is between about 80 mg to about 300 mg. In some aspects, the C1D3 is about 90 mg. In some aspects, the C1D3 is about 132 mg. In some aspects, the C1D3 is about 160 mg.

In some aspects, the C1D3 is between 20 mg to 600 mg (e.g., between 30 mg to 500 mg, 40 mg to 400 mg, 60 mg to 350 mg, 80 mg to 300 mg, 100 mg to 200 mg, or 140 mg to 180 mg, e.g., 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, or 600 mg). In some aspects, the C1D3 is between 80 mg to 300 mg. In some aspects, the C1D3 is 40 mg. In some aspects, the C1D3 is 90 mg. In some aspects, the C1D3 is 120 mg. In some aspects, the C1D3 is 132 mg. In some aspects, the C1D3 is 160 mg. In some aspects, the C1D3 is 198 mg. In some aspects, the C1D3 is 252 mg.

In some aspects, the method comprises only a single dosing cycle of the bispecific antibody (e.g., a dosing cycle comprising a C1D1, a C1D2, and a C1D3).

In other aspects, the dosing regimen further comprises a second dosing cycle comprising at least a single dose (C2D1) of the bispecific antibody. In some aspects, the C2D1 is equal to or greater than the C1D3 and is between about 20 mg to about 600 mg (e.g., between about 30 mg to about 500 mg, about 40 mg to about 400 mg, about 60 mg to about 350 mg, about 80 mg to about 300 mg, about 100 mg to about 200 mg, or about 140 mg to about 180 mg, e.g., about 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, or 600 mg). In some aspects, the C2D1 is between about 80 mg to about 300 mg. In some aspects, the C2D1 is about 40 mg. In some aspects, the C2D1 is about 90 mg. In some aspects, the C2D1 is about 120 mg. In some aspects, the C2D1 is about 132 mg. In some aspects, the C2D1 is about 160 mg. In some aspects, the C2D1 is about 252 mg. In some aspects, the C2D1 is about 252 mg.

In some aspects, the C2D1 is between 20 mg to 600 mg (e.g., between 30 mg to 500 mg, 40 mg to 400 mg, 60 mg to 350 mg, 80 mg to 300 mg, 100 mg to 200 mg, or 140 mg to 180 mg, e.g., 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, or 600 mg). In some aspects, the C2D1 is between 80 mg to 300 mg. In some aspects, the C2D1 is 40 mg. In some aspects, the C2D1 is 90 mg. In some aspects, the C2D1 is 120 mg. In some aspects, the C2D1 is 132 mg. In some aspects, the C2D1 is 160 mg. In some aspects, the C2D1 is 198 mg. In some aspects, the C2D1 is 252 mg.

Alternatively, in any of the above embodiments, the C1D1 may be between about 0.01 mg to about 60 mg (e.g., between about 0.05 mg to about 50 mg, between about 0.01 mg to about 40 mg, between about 0.1 mg to about 20 mg, between about 0.1 mg to about 10 mg, between about 0.1 mg to about 5 mg, between about 0.1 mg to about 2 mg, between about 0.1 mg to about 1.5 mg, between about 0.1 mg to about 1.2 mg, between about 0.1 mg to about 0.5 mg, or between about 0.2 mg to about 0.4 mg, e.g., about 0.3 mg, e.g., 0.3 mg), the C1D2 may be between about 0.05 mg to about 180 mg (e.g., between about 0.1 mg to about 160 mg, between about 0.5 mg to about 140 mg, between about 1 mg to about 120 mg, between about 1.5 mg to about 100 mg, between about 2.0 mg to about 80 mg, between about 2.5 mg to about 50 mg, between about 3.0 mg to about 25 mg, between about 3.0 mg to about 15 mg, between about 3.0 mg to about 10 mg, between about 3.0 mg to about 5 mg, or between about 3.0 mg to about 4.0 mg, e.g., about 3.6 mg or about 3.3 mg, e.g., 3.6 mg or 3.3 mg), and the C1D3 may be between about 0.15 mg to about 1000 mg (e.g., between about 0.5 mg to about 800 mg, between about 1 mg to about 700 mg, between about 5 mg to about 500 mg, between about 10 mg to about 400 mg, between about 25 mg to about 300 mg, between about 40 mg to about 200 mg, between about 50 mg to about 190 mg, between about 140 mg to about 180 mg, or between about 150 mg to about 170 mg, e.g., about 160 mg, e.g., 160 mg); and in aspects comprising a second dosing cycle, the C2D1 may be between about 0.15 mg to about 1000 mg (e.g., between about 0.5 mg to about 800 mg, between about 1 mg to about 700 mg, between about 5 mg to about 500 mg, between about 10 mg to about 400 mg, between about 25 mg to about 300 mg, between about 40 mg to about 200 mg, between about 50 mg to about 190 mg, between about 140 mg to about 180 mg, or between about 150 mg to about 170 mg, e.g., about 160 mg, e.g., 160 mg).

In some instances, the length of the first dosing cycle is one week or 7 days. In some instances, the length of the first dosing cycle is two weeks or 14 days. In some instances, the length of the first dosing cycle is three weeks or 21 days. In some instances, the length of the first dosing cycle four weeks or 28 days.

In any of the examples described herein, the first step-up dose and the second step-up dose may be administered about one day apart (e.g., about 20 hours apart, about 21 hours apart, about 23 hours apart, about 24 hours apart, about 25 hours apart, about 26 hours apart, about 27 hours apart, or about 28 hours apart), about two days apart (e.g., about 44 hours apart, about 45 hours apart, about 46 hours apart, about 47 hours apart, about 48 hours apart, about 49 hours apart, about 50 hours apart, about 51 hours apart, or about 52 hours apart), or about 3 days apart (e.g., about 68 hours apart, about 69 hours apart, about 70 hours apart, about 71 hours apart, or about 72 hours apart).

For example, in some instances, the methods may include administering to the subject the C1D1, the C1D2, and the C1D3 on or about Days 1, 2, and 8, respectively, of the first dosing cycle.

In other instances, the methods may include administering to the subject the C1D1, the C1D2, and the C1D3 on or about Days 1, 3, and 8, respectively, of the first dosing cycle (e.g., in the event of an unwanted treatment effect on Day 1, such as a CRS, IRR, MAS, or TLS).

In other instances, the methods may include administering to the subject the C1D1, the C1D2, and the C1D3 on or about Days 1, 4, and 8, respectively, of the first dosing cycle (e.g., in the event of an unwanted treatment effect on Day 1, such as a CRS, IRR, MAS, or TLS).

In some instances, the methods may include administering to the subject the C1D3 on or after Day 9 (e.g., Day 9, Day 10, Day 11, Day 12, Day 13, Day 14, Day 15, Day 16, Day 17, Day 18, Day 19, Day 20, or Day 21) of the first dosing cycle.

In other instances, the methods may include administering to the subject the C1D1, the C1D2, and the C1D3 on or about Days 1, 2, and 9, respectively, of the first dosing cycle (e.g., in the event of an unwanted treatment effect on Day 1, such as a CRS, IRR, MAS, or TLS).

In other instances, the methods may include administering to the subject the C1D1, the C1D2, and the C1D3 on or about Days 1, 3, and 9, respectively, of the first dosing cycle (e.g., in the event of an unwanted treatment effect on Day 1, such as a CRS, IRR, MAS, or TLS).

In other instances, the methods may include administering to the subject the C1D1, the C1D2, and the C1D3 on or about Days 1, 4, and 9, respectively, of the first dosing cycle (e.g., in the event of an unwanted treatment effect on Day 1, such as a CRS, IRR, MAS, or TLS).

In other instances, the methods may include administering to the subject the C1D1, the C1D2, and the C1D3 on or about Days 1, 5, and 8, respectively, of the first dosing cycle.

In other instances, the methods may include administering to the subject the C1D1, the C1D2, and the C1D3 on or about Days 1, 6, and 8, respectively, of the first dosing cycle.

In other instances, the methods may include administering to the subject the C1D1, the C1D2, and the C1D3 on or about Days 1, 7, and 8, respectively, of the first dosing cycle.

In yet other instances, the methods may include administering to the subject the C1D1, the C1D2, and the C1D3 on or about Days 2, 3, and 8, respectively, of the first dosing cycle.

In other instances, the methods may include administering to the subject the C1D1, the C1D2, and the C1D3 on or about Days 3, 4, and 8, respectively, of the first dosing cycle.

iii. Further Dosing Cycles

Any of the methods disclosed herein, including any of the single step-up or double step-up dosing regimens disclosed above, may include any suitable number of further dosing cycles. In some instances, the methods described above may include a second dosing cycle of one week or 7 days. In some instances, the methods described above may include a second dosing cycle of two weeks or 14 days. In some instances, the methods described above may include a second dosing cycle of three weeks or 21 days. In some instances, the methods described above may include a second dosing cycle of four weeks or 28 days. In some instances, the methods may include administering to the subject the C2D1 on or about Day 1 of the second dosing cycle. In some instances, the methods include administering to the subject the C2D1 at least one week (7 days) after administration of a previous dose (e.g., the C1D3) of the bispecific antibody.

In some instances, in which the methods include at least a second dosing cycle, the methods may include one or more additional dosing cycles. In some instances, the dosing regimen comprises 1 to 17 additional dosing cycles (e.g., 1, 2, 3, 4, 5, 6, 7, 8, 9, 10, 11, 12, 13, 14, 15, 16, or 17 additional dosing cycles, e.g., 1-3 additional dosing cycles, 1-5 additional dosing cycles, 3-8 additional dosing cycles, 5-10 additional dosing cycles, 8-12 additional dosing cycles, 10-15 additional dosing cycles, 12-17 additional dosing cycles, or 15-17 additional dosing cycles, i.e., the dosing regimen includes one or more of additional dosing cycle(s) C3, C4, C5, C6, C7, C8, C9, C10, C11, C12, C13, C14, C15, C16, C17, C18, and C19.

In some instances, in which the methods include at least a second dosing cycle, the methods may include any suitable number of additional dosing cycles. For example, the additional dosing cycles may continue until the subject experiences disease progression, unacceptable toxicity, or death.

In some embodiments, the length of each of the one or more additional dosing cycles is 7 days, 14 days, 21 days, or 28 days. In some embodiments, the length of each of the one or more additional dosing cycles is between 5 days and 30 days, e.g., between 5 and 9 days, between 7 and 11 days, between 9 and 13 days, between 11 and 15 days, between 13 and 17 days, between 15 and 19 days, between 17 and 21 days, between 19 and 23 days, between 21 and 25 days, between 23 and 27 days, or between 25 and 30 days. In some instances, the length of each of the one or more additional dosing cycles is one week or 7 days (e.g., Q1W). In some instances, the length of each of the one or more additional dosing cycles is two weeks or 14 days (e.g., Q2W). In some instances, the length of each of the one or more additional dosing cycles is three weeks or 21 days (e.g., Q3W). In some instances, the length of each of the one or more additional dosing cycles is four weeks or 28 days (e.g., Q4W).

In some instances, each of the one or more additional dosing cycles comprises a single dose of the bispecific antibody. In some aspects, the dose of the bispecific antibody in the one or more additional dosing cycles is equal to the C2D1, e.g., is between about 20 mg to about 600 mg (e.g., between about 30 mg to about 500 mg, about 40 mg to about 400 mg, about 60 mg to about 350 mg, about 80 mg to about 300 mg, about 100 mg to about 200 mg, or about 140 mg to about 180 mg, e.g., about 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, or 600 mg). In some aspects, the dose of the bispecific antibody in the one or more additional dosing cycles is about 40 mg. In some aspects, the dose of the bispecific antibody in the one or more additional dosing cycles is about 90 mg. In some aspects, the dose of the bispecific antibody in the one or more additional dosing cycles is about 120 mg. In some aspects, the dose of the bispecific antibody in the one or more additional dosing cycles is about 132 mg. In some aspects, the dose of the bispecific antibody in the one or more additional dosing cycles is about 160 mg. In some aspects, the dose of the bispecific antibody in the one or more additional dosing cycles is about 198 mg. In some aspects, the dose of the bispecific antibody in the one or more additional dosing cycles is about 252 mg.

In some aspects, the dose of the bispecific antibody in the one or more additional dosing cycles is equal to the C2D1, e.g., is between 20 mg to 600 mg (e.g., between 30 mg to 500 mg, 40 mg to 400 mg, 60 mg to 350 mg, 80 mg to 300 mg, 100 mg to 200 mg, or 140 mg to 180 mg, e.g., 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, or 600 mg). In some aspects, the dose of the bispecific antibody in the one or more additional dosing cycles is 40 mg. In some aspects, the dose of the bispecific antibody in the one or more additional dosing cycles is 90 mg. In some aspects, the dose of the bispecific antibody in the one or more additional dosing cycles is 120 mg. In some aspects, the dose of the bispecific antibody in the one or more additional dosing cycles is 132 mg. In some aspects, the dose of the bispecific antibody in the one or more additional dosing cycles is about 160 mg. In some aspects, the dose of the bispecific antibody in the one or more additional dosing cycles is 198 mg. In some aspects, the dose of the bispecific antibody in the one or more additional dosing cycles is 252 mg.

In some instances, the method comprises administering to the subject the single dose of the bispecific antibody on or about Day 1 of the one or more additional dosing cycles. In some instances, the method comprises administering to the subject the single dose of the bispecific antibody on or about Day 1 and 15 of the one or more additional dosing cycles. In some instances, the method comprises administering to the subject the single dose of the bispecific antibody on or about Day 1, 8, 15, and 22 of the one or more additional dosing cycles.

In some aspects, the bispecific antibody is administered to the subject every 7 days (QW) until progressive disease is observed, for up to 18 cycles, or until minimal residual disease (MRD) is observed. In some aspects, the bispecific antibody is administered to the subject every 14 days (Q2W) until progressive disease is observed, for up to 18 cycles, or until minimal residual disease (MRD) is observed. In some aspects, the bispecific antibody is administered to the subject every 21 days (Q3W) until progressive disease is observed, for up to 18 cycles, or until minimal residual disease (MRD) is observed. In some aspects, the bispecific antibody is administered to the subject every 28 days (Q4W) until progressive disease is observed, for up to 18 cycles, or until minimal residual disease (MRD) is observed. In some aspects, the bispecific antibody is administered to the subject QW, Q2W, Q3W, or Q4W until disease progression, unacceptable toxicity, or death is observed.

In some instances, the bispecific anti-FcRH5/anti-CD3 antibody is administered to the subject as a monotherapy. In some instances, the bispecific anti-FcRH5/anti-CD3 antibody is administered to the subject in combination with another therapeutic agent. In some instances, the bispecific anti-FcRH5/anti-CD3 antibody is administered to the subject in combination with a corticosteroid. Exemplary corticosteroids to be used in combination therapy include dexamethasone and methylprednisolone.

In some instances, the bispecific anti-FcRH5/anti-CD3 antibody is cevostamab. In some instances, cevostamab is administered to the subject as a monotherapy. In some instances, cevostamab is administered to the subject in combination with a corticosteroid (e.g., dexamethasone and methylprednisolone).

### B. Dosing Regimens

The present disclosure describes a method of treating a subject having a cancer (e.g., a multiple myeloma (MM)), the method comprising administering to the subject a bispecific antibody that binds to FcRH5 and CD3 in a dosing regimen described herein. In some examples, the subject has a triple-class refractory MM and has previously received a BCMA-targeting therapeutic agent.

In some examples, the dosing regimen comprises a first phase comprising one or more dosing cycles and a second phase comprising one or more dosing cycles. In some examples, each dosing cycle is a 7-day dosing cycle. In some examples, each dosing cycle is a 14-day dosing cycle. In some examples, each dosing cycle is a 21-day dosing cycle. In some examples, each dosing cycle is a 28-day dosing cycle. In one example, the first phase may include administering the bispecific antibody to the subject on Day 1, Day 2, and Day 8 of each dosing cycle of the first phase and the second phase may include administering the bispecific antibody to the subject QW, Q2W, Q3W, or Q4W. In other example, the first phase may include administering the bispecific antibody to the subject on Day 1, Day 3, and/or Day 8 of each dosing cycle of the first phase and the second phase may include administering the bispecific antibody to the subject QW, Q2W, Q3W, or Q4W. In yet another example, the first phase may include administering the bispecific antibody to the subject on Day 1, Day 4, and/or Day 8 of each dosing cycle of the first phase and the second phase may include administering the bispecific antibody to the subject QW, Q2W, Q3W, or Q4W. In other example, the first phase may include administering the bispecific antibody to the subject on Day 1, Day 3, and/or Day 9 of each dosing cycle of the first phase and the second phase may include administering the bispecific antibody to the subject QW, Q2W, Q3W, or Q4W. In yet another example, the first phase may include administering the bispecific antibody to the subject on Day 1, Day 4, and/or Day 9 of each dosing cycle of the first phase and the second phase may include administering the bispecific antibody to the subject QW, Q2W, Q3W, or Q4W.

For example, provided herein is a method of treating a subject having a cancer (e.g., an MM), the method comprising administering to the subject a bispecific antibody that binds to FcRH5 and CD3 in a dosing regimen comprising: (i) a first phase comprising one or more dosing cycles, wherein the first phase comprises administering the bispecific antibody to the subject on (a) Day 1, (b) Day 2, Day 3, or Day 4, and/or (c) Day 8 or Day 9 of each dosing cycle of the first phase; and (ii) a second phase comprising one or more dosing cycles, wherein the second phase comprises administering the bispecific antibody to the subject every three weeks (Q3W). In some examples, the dosing regimen includes the first phase. In some examples, the dosing regimen includes the second phase. In some examples, the dosing regimen includes the first phase and the second phase.

In another example, provided herein is a bispecific antibody that binds to FcRH5 and CD3 for use in treatment of a subject having a cancer (e.g., an MM), the treatment comprising administration of the bispecific antibody to the subject in a dosing regimen comprising: (i) a first phase comprising one or more dosing cycles, wherein the first phase comprises administering the bispecific antibody to the subject on (a) Day 1, (b) Day 2, Day 3, or Day 4, and/or (c) Day 8 or Day 9 of each dosing cycle of the first phase; and (ii) a second phase comprising one or more dosing cycles, wherein the second phase comprises administering the bispecific antibody to the subject every three weeks (Q3W). In some examples, the dosing regimen includes the first phase. In some examples, the dosing regimen includes the second phase. In some examples, the dosing regimen includes the first phase and the second phase.

In another example, provided herein is the use of a bispecific antibody that binds to FcRH5 and CD3 in the manufacture of a medicament for treatment of a subject having a cancer (e.g., an MM), the treatment comprising administration of the bispecific antibody to the subject in a dosing regimen comprising: (i) a first phase comprising one or more dosing cycles, wherein the first phase comprises administering the bispecific antibody to the subject on (a) Day 1, (b) Day 2, Day 3, or Day 4, and/or (c) Day 8 or Day 9 of each dosing cycle of the first phase; and (ii) a second phase comprising one or more dosing cycles, wherein the second phase comprises administering the bispecific antibody to the subject every three weeks (Q3W). In some examples, the dosing regimen includes the first phase. In some examples, the dosing regimen includes the second phase. In some examples, the dosing regimen includes the first phase and the second phase.

In another example, provided herein is a method of treating a subject having a cancer (e.g., an MM), the method comprising administering to the subject a bispecific antibody that binds to FcRH5 and CD3 in a dosing regimen comprising: (i) a first phase comprising one or more dosing cycles, wherein the first phase comprises administering the bispecific antibody to the subject on (a) Day 1, Day 2, Day 3, Day 4, Day 5, Day 6, and/or Day 7 of each dosing cycle of the first phase and/or (b) on Day 8 or Day 9 of each dosing cycle of the first phase; and (ii) a second phase comprising one or more dosing cycles, wherein the second phase comprises administering the bispecific antibody to the subject every three weeks (Q3W). In some examples, the dosing regimen includes the first phase. In some examples, the dosing regimen includes the second phase. In some examples, the dosing regimen includes the first phase and the second phase. In some examples, if the subject experiences a CRS event following administration of a first step-up dose (e.g., 0.3 mg) during the first phase on Day 1 of C1, administration of the second dose (e.g., 3.3 mg) may be delivered on Day 2, Day 3, or Day 4, upon full resolution of CRS.

In another example, provided herein is a bispecific antibody that binds to FcRH5 and CD3 for use in treatment of a subject having a cancer (e.g., an MM), the treatment comprising administration of the bispecific antibody to the subject in a dosing regimen comprising: (i) a first phase comprising one or more dosing cycles, wherein the first phase comprises administering the bispecific antibody to the subject on (a) Day 1, Day 2, Day 3, Day 4, Day 5, Day 6, and/or Day 7 of each dosing cycle of the first phase and/or (b) on Day 8 or Day 9 of each dosing cycle of the first phase; and (ii) a second phase comprising one or more dosing cycles, wherein the second phase comprises administering the bispecific antibody to the subject every three weeks (Q3W). In some examples, the dosing regimen includes the first phase. In some examples, the dosing regimen includes the second phase. In some examples, the dosing regimen includes the first phase and the second phase. In some examples, if the subject experiences a CRS event following administration of a first step-up dose (e.g., 0.3 mg) during the first phase on Day 1 of C1, administration of the second dose (e.g., 3.3 mg) may be delivered on Day 2, Day 3, or Day 4, upon full resolution of CRS.

In another example, provided herein is the use of a bispecific antibody that binds to FcRH5 and CD3 in the manufacture of a medicament for treatment of a subject having a cancer (e.g., an MM), the treatment comprising administration of the bispecific antibody to the subject in a dosing regimen comprising: (i) a first phase comprising one or more dosing cycles, wherein the first phase comprises administering the bispecific antibody to the subject on (a) Day 1, Day 2, Day 3, Day 4, Day 5, Day 6, and/or Day 7 of each dosing cycle of the first phase and/or (b) on Day 8 or Day 9 of each dosing cycle of the first phase; and (ii) a second phase comprising one or more dosing cycles, wherein the second phase comprises administering the bispecific antibody to the subject every three weeks (Q3W). In some examples, the dosing regimen includes the first phase. In some examples, the dosing regimen includes the second phase. In some examples, the dosing regimen includes the first phase and the second phase. In some examples, if the subject experiences a CRS event following administration of a first step-up dose (e.g., 0.3 mg) during the first phase on Day 1 of C1, administration of the second dose (e.g., 3.3 mg) may be delivered on Day 2, Day 3, or Day 4, upon full resolution of CRS.

The first phase may comprise any suitable number of dosing cycles. For example, in some examples, first phase may comprise one dosing cycle, at least two dosing cycles, at least three dosing cycles, at least four dosing cycle, at least five dosing cycles, at least six dosing cycles, at least seven, at least eight dosing cycle, at least nine dosing cycle, at least ten dosing cycle, at least eleven dosing cycles, at least twelve dosing cycles, or at least thirteen dosing cycles, or more.

In some examples, first phase comprises a first dosing cycle (C1); a first dosing cycle and a second dosing cycle (C2); a first dosing cycle, a second dosing cycle (C2), and a third dosing cycle (C3); a first dosing cycle (C1), a second dosing cycle (C2), a third dosing cycle (C3), and a fourth dosing cycle (C4); a first dosing cycle (C1), a second dosing cycle (C2), a third dosing cycle (C3), a fourth dosing cycle (C4), and a fifth dosing cycle (C5); a first dosing cycle (C1), a second dosing cycle (C2), a third dosing cycle (C3), a fourth dosing cycle (C4), a fifth dosing cycle (C5), and a sixth dosing cycle (C6); a first dosing cycle (C1), a second dosing cycle (C2), a third dosing cycle (C3), a fourth dosing cycle (C4), a fifth dosing cycle (C5), a sixth dosing cycle (C6), and a seventh dosing cycle (C7); a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), and an eighth dosing cycle (C8); a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), and a ninth dosing cycle (C9); a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), and a tenth dosing cycle (C10); a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), and an eleventh dosing cycle (C11); a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), and a twelfth dosing cycle (C12); or a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), and a thirteenth dosing cycle (C13).

The bispecific antibody may be administered on any suitable day of a given dosing cycle. For example, for a 28-day dosing cycle, the bispecific antibody may be administered on Day 1, 2, 3, 4, 5, 6, 7, 8, 9, 10, 11, 12, 13, 14, 15, 16, 17, 18, 19, 20, 21, 22, 23, 24, 25, 26, 27, or 28. In another example, for a 21-day dosing cycle, the bispecific antibody may be administered on Day 1, 2, 3, 4, 5, 6, 7, 8, 9, 10, 11, 12, 13, 14, 15, 16, 17, 18, 19, 20, or 21. In another example, for a 14-day dosing cycle, the bispecific antibody may be administered on Day 1, 2, 3, 4, 5, 6, 7, 8, 9, 10, 11, 12, 13, or 14. In another example, for a 7-day dosing cycle, the bispecific antibody may be administered on Day 1, 2, 3, 4, 5, 6, or 7.

In some examples, the first phase comprises administration of the bispecific antibody to the subject on Days 1, 2, and/or 8 of C1. In a further example, the first phase comprises administration of the bispecific antibody to the subject on Days 1, 2, and/or 8 of C2. In a further example, the first phase comprises administration of the bispecific antibody to the subject on Days 1, 2, and/or 8 of C3. In a further example, the first phase comprises administration of the bispecific antibody to the subject on Days 1, 2, and/or 8 of C4. In a further example, the first phase comprises administration of the bispecific antibody to the subject on Days 1, 2, and/or 8 of C5. In a further example, the first phase comprises administration of the bispecific antibody to the subject on Days 1, 2, and/or 8 of C6. In a further example, the first phase comprises administration of the bispecific antibody to the subject on Days 1, 2, and/or 8 of C7. In a further example, the first phase comprises administration of the bispecific antibody to the subject on Days 1, 2, and/or 8 of C8. In a further example, the first phase comprises administration of the bispecific antibody to the subject on Days 1, 2, and/or 8 of C9. In a further example, the first phase comprises administration of the bispecific antibody to the subject on Days 1, 2, and/or 8 of C10. In a further example, the first phase comprises administration of the bispecific antibody to the subject on Days 1, 2, and/or 8 of C11. In a further example, the first phase comprises administration of the bispecific antibody to the subject on Days 1, 2, and/or 8 of C12. In a further example, the first phase comprises administration of the bispecific antibody to the subject on Days 1, 2, and/or 8 of C13.

In some examples, the first phase comprises administration of the bispecific antibody to the subject on Days 1, 3, and/or 8 of C1. In a further example, the first phase comprises administration of the bispecific antibody to the subject on Days 1, 3, and/or 8 of C2. In a further example, the first phase comprises administration of the bispecific antibody to the subject on Days 1, 3, and/or 8 of C3. In a further example, the first phase comprises administration of the bispecific antibody to the subject on Days 1, 3, and/or 8 of C4. In a further example, the first phase comprises administration of the bispecific antibody to the subject on Days 1, 3, and/or 8 of C5. In a further example, the first phase comprises administration of the bispecific antibody to the subject on Days 1, 3, and/or 8 of C6. In a further example, the first phase comprises administration of the bispecific antibody to the subject on Days 1, 3, and/or 8 of C7. In a further example, the first phase comprises administration of the bispecific antibody to the subject on Days 1, 3, and/or 8 of C8. In a further example, the first phase comprises administration of the bispecific antibody to the subject on Days 1, 3, and/or 8 of C9. In a further example, the first phase comprises administration of the bispecific antibody to the subject on Days 1, 3, and/or 8 of C10. In a further example, the first phase comprises administration of the bispecific antibody to the subject on Days 1, 3, and/or 8 of C11. In a further example, the first phase comprises administration of the bispecific antibody to the subject on Days 1, 3, and/or 8 of C12. In a further example, the first phase comprises administration of the bispecific antibody to the subject on Days 1, 3, and/or 8 of C13.

In some examples, the first phase comprises administration of the bispecific antibody to the subject on Days 1, 4, and/or 8 of C1. In a further example, the first phase comprises administration of the bispecific antibody to the subject on Days 1, 4, and/or 8 of C2. In a further example, the first phase comprises administration of the bispecific antibody to the subject on Days 1, 4, and/or 8 of C3. In a further example, the first phase comprises administration of the bispecific antibody to the subject on Days 1, 4, and/or 8 of C4. In a further example, the first phase comprises administration of the bispecific antibody to the subject on Days 1, 4, and/or 8 of C5. In a further example, the first phase comprises administration of the bispecific antibody to the subject on Days 1, 4, and/or 8 of C6. In a further example, the first phase comprises administration of the bispecific antibody to the subject on Days 1, 4, and/or 8 of C7. In a further example, the first phase comprises administration of the bispecific antibody to the subject on Days 1, 4, and/or 8 of C8. In a further example, the first phase comprises administration of the bispecific antibody to the subject on Days 1, 4, and/or 8 of C9. In a further example, the first phase comprises administration of the bispecific antibody to the subject on Days 1, 4, and/or 8 of C10. In a further example, the first phase comprises administration of the bispecific antibody to the subject on Days 1, 4, and/or 8 of C11. In a further example, the first phase comprises administration of the bispecific antibody to the subject on Days 1, 4, and/or 8 of C12. In a further example, the first phase comprises administration of the bispecific antibody to the subject on Days 1, 4, and/or 8 of C13.

In some examples, the first phase comprises administration of the bispecific antibody to the subject on Days 1, 2, and/or 9 of C1. In a further example, the first phase comprises administration of the bispecific antibody to the subject on Days 1, 2, and/or 9 of C2. In a further example, the first phase comprises administration of the bispecific antibody to the subject on Days 1, 2, and/or 9 of C3. In a further example, the first phase comprises administration of the bispecific antibody to the subject on Days 1, 2, and/or 9 of C4. In a further example, the first phase comprises administration of the bispecific antibody to the subject on Days 1, 2, and/or 9 of C5. In a further example, the first phase comprises administration of the bispecific antibody to the subject on Days 1, 2, and/or 9 of C6. In a further example, the first phase comprises administration of the bispecific antibody to the subject on Days 1, 2, and/or 9 of C7. In a further example, the first phase comprises administration of the bispecific antibody to the subject on Days 1, 2, and/or 9 of C9. In a further example, the first phase comprises administration of the bispecific antibody to the subject on Days 1, 2, and/or 9 of C9. In a further example, the first phase comprises administration of the bispecific antibody to the subject on Days 1, 2, and/or 9 of C10. In a further example, the first phase comprises administration of the bispecific antibody to the subject on Days 1, 2, and/or 9 of C11. In a further example, the first phase comprises administration of the bispecific antibody to the subject on Days 1, 2, and/or 9 of C12. In a further example, the first phase comprises administration of the bispecific antibody to the subject on Days 1, 2, and/or 9 of C13.

In some examples, the first phase comprises administration of the bispecific antibody to the subject on Days 1, 3, and/or 9 of C1. In a further example, the first phase comprises administration of the bispecific antibody to the subject on Days 1, 3, and/or 9 of C2. In a further example, the first phase comprises administration of the bispecific antibody to the subject on Days 1, 3, and/or 9 of C3. In a further example, the first phase comprises administration of the bispecific antibody to the subject on Days 1, 3, and/or 9 of C4. In a further example, the first phase comprises administration of the bispecific antibody to the subject on Days 1, 3, and/or 9 of C5. In a further example, the first phase comprises administration of the bispecific antibody to the subject on Days 1, 3, and/or 9 of C6. In a further example, the first phase comprises administration of the bispecific antibody to the subject on Days 1, 3, and/or 9 of C7. In a further example, the first phase comprises administration of the bispecific antibody to the subject on Days 1, 3, and/or 9 of C9. In a further example, the first phase comprises administration of the bispecific antibody to the subject on Days 1, 3, and/or 9 of C9. In a further example, the first phase comprises administration of the bispecific antibody to the subject on Days 1, 3, and/or 9 of C10. In a further example, the first phase comprises administration of the bispecific antibody to the subject on Days 1, 3, and/or 9 of C11. In a further example, the first phase comprises administration of the bispecific antibody to the subject on Days 1, 3, and/or 9 of C12. In a further example, the first phase comprises administration of the bispecific antibody to the subject on Days 1, 3, and/or 9 of C13.

In some examples, the first phase comprises administration of the bispecific antibody to the subject on Days 1, 4, and/or 9 of C1. In a further example, the first phase comprises administration of the bispecific antibody to the subject on Days 1, 4, and/or 9 of C2. In a further example, the first phase comprises administration of the bispecific antibody to the subject on Days 1, 4, and/or 9 of C3. In a further example, the first phase comprises administration of the bispecific antibody to the subject on Days 1, 4, and/or 9 of C4. In a further example, the first phase comprises administration of the bispecific antibody to the subject on Days 1, 4, and/or 9 of C5. In a further example, the first phase comprises administration of the bispecific antibody to the subject on Days 1, 4, and/or 9 of C6. In a further example, the first phase comprises administration of the bispecific antibody to the subject on Days 1, 4, and/or 9 of C7. In a further example, the first phase comprises administration of the bispecific antibody to the subject on Days 1, 4, and/or 9 of C9. In a further example, the first phase comprises administration of the bispecific antibody to the subject on Days 1, 4, and/or 9 of C9. In a further example, the first phase comprises administration of the bispecific antibody to the subject on Days 1, 4, and/or 9 of C10. In a further example, the first phase comprises administration of the bispecific antibody to the subject on Days 1, 4, and/or 9 of C11. In a further example, the first phase comprises administration of the bispecific antibody to the subject on Days 1, 4, and/or 9 of C12. In a further example, the first phase comprises administration of the bispecific antibody to the subject on Days 1, 4, and/or 9 of C13.

In some examples, a target dose of the bispecific antibody is administered to the subject on or after Day 9 of the C1, C2, C3, C4, C5, C6, C7, C8, C9, C10, C11, or C12 of the first phase. For example, in some instances, a target dose of the bispecific antibody is administered to the subject on Day 9, Day 10, Day 11, Day 12, Day 13, Day 14, Day 15, Day 16, Day 17, Day 18, Day 19, Day 20, or Day 21 of the C1, C2, C3, C4, C5, C6, C7, C8, C9, C10, C11, or C12 of the first phase.

In some examples, a target dose of the bispecific antibody is administered to the subject for each administration during the first phase.

In some examples, the first phase comprises administration of a target dose of the bispecific antibody to the subject on Day 1, 2, 3, 4, 5, 6, 7, or 8 of C1. In a further example, the first phase comprises administration of the target dose of the bispecific antibody to the subject on Day 1, 2, 3, 4, 5, 6, 7, or 8 of C2. In a further example, the first phase comprises administration of the target dose of the bispecific antibody to the subject on Day 1, 2, 3, 4, 5, 6, 7, or 8 of C3. In a further example, the first phase comprises administration of the target dose of the bispecific antibody to the subject on Day 1, 2, 3, 4, 5, 6, 7, or 8 of C4. In a further example, the first phase comprises administration of the target dose of the bispecific antibody to the subject on Day 1, 2, 3, 4, 5, 6, 7, or 8 of C5. In a further example, the first phase comprises administration of the target dose of the bispecific antibody to the subject on Day 1, 2, 3, 4, 5, 6, 7, or 8 of C6. In a further example, the first phase comprises administration of the target dose of the bispecific antibody to the subject on Day 1, 2, 3, 4, 5, 6, 7, or 8 of C7. In a further example, the first phase comprises administration of the target dose of the bispecific antibody to the subject on Day 1, 2, 3, 4, 5, 6, 7, or 8 of C8. In a further example, the first phase comprises administration of the target dose of the bispecific antibody to the subject on Day 1, 2, 3, 4, 5, 6, 7, or 8 of C9. In a further example, the first phase comprises administration of the target dose of the bispecific antibody to the subject on Day 1, 2, 3, 4, 5, 6, 7, or 8 of C10. In a further example, the first phase comprises administration of the target dose of the bispecific antibody to the subject on Day 1, 2, 3, 4, 5, 6, 7, or 8 of C11. In a further example, the first phase comprises administration of the target dose of the bispecific antibody to the subject on Day 1, 2, 3, 4, 5, 6, 7, or 8 of C12. In a further example, the first phase comprises administration of the target dose of the bispecific antibody to the subject on Day 1, 2, 3, 4, 5, 6, 7, or 8 of C13.

In some examples, the first phase comprises administration of a first step-up dose and a target dose of the bispecific antibody to the subject. The first step-up dose may be administered to the subject during the first phase on Day 1 of C1, on Day 2 of C1, on Day 3 of C1, on Day 4 of C1, on Day 5 of C1, on Day 6 of C1, or on Day 7 of C1. The target dose may be administered to the subject during the first phase on Day 8 of C1. In a further example, the target dose may be administered to the subject during the first phase on Day 1 of C2. In a further example, the target dose may be administered to the subject during the first phase on Day 1 of C3. In a further example, the target dose may be administered to the subject during the first phase on Day 1 of C4. In a further example, the target dose may be administered to the subject during the first phase on Day 1 of C5. In a further example, the target dose may be administered to the subject during the first phase on Day 1 of C6. In a further example, the target dose may be administered to the subject during the first phase on Day 1 of C7. In a further example, the target dose may be administered to the subject during the first phase on Day 1 of C8. In a further example, the target dose may be administered to the subject during the first phase on Day 1 of C9. In a further example, the target dose may be administered to the subject during the first phase on Day 1 of C10. In a further example, the target dose may be administered to the subject during the first phase on Day 1 of C11. In a further example, the target dose may be administered to the subject during the first phase on Day 1 of C12. In a further example, the target dose may be administered to the subject during the first phase on Day 1 of C13.

In some examples, the first step-up dose is about 0.1% to about 8% of the target dose. In some examples, the first step-up dose is about 0.12%, about 0.13%, about 0.14%, about 0.15%, about 0.16%, about 0.17%, about 0.18%, about 0.19%, about 0.2%, about 0.21%, about 0.22%, about 0.23%, about 0.24%, about 0.25%, about 0.26%, about 0.27%, about 0.28%, about 0.29%, about 0.3%, about 0.4%, about 0.5%, about 0.6%, about 0.7%, about 0.8%, about 0.9%, about 1%, about 1.5%, about 2%, about 2.03%, about 2.1%, about 2.2%, about 2.3%, about 2.4%, about 2.5%, about 3%, about 3.5%, about 4%, about 4.5%, about 5%, about 5.5%, about 6%, about 6.5%, about 7%, about 7.5%, or about 8% of the target dose.

In some examples, the first step-up dose is 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.4%, 0.5%, 0.6%, 0.7%, 0.8%, 0.9%, 1%, 1.5%, 2%, 2.03%, 2.1%, 2.2%, 2.3%, 2.4%, 2.5%, 3%, 3.5%, 4%, 4.5%, 5%, 5.5%, 6%, 6.5%, 7%, 7.5%, or 8% of the target dose. In some examples, the first step-up dose is 4% of the target dose.

In some examples, the first step-up dose is about 3.3 mg. In some examples, the first step-up dose is about 3.6 mg. In some examples, the first step-up dose is about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, or about 10 mg.

In some examples, the first step-up dose is 3.3 mg. In some examples, the first step-up dose is 3.6 mg. In some examples, the first step-up dose is 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, or 10 mg.

In some examples, the first phase comprises administration of a first step-up dose and a second step-up dose of the bispecific antibody to the subject. In some examples, the first step-up dose is administered to the subject during the first phase on Day 1 of C1 while the second step-up dose is administered on Day 2 of C1. In some examples, the first step-up dose is administered to the subject during the first phase on Day 1 of C1 while the second step-up dose is administered on Day 3 of C1. In some examples, the first step-up dose is administered to the subject during the first phase on Day 1 of C1 while the second step-up dose is administered on Day 4 of C1. In some examples, the first step-up dose is administered to the subject during the first phase on Day 1 of C1 while the second step-up dose is administered on Day 5 of C1. In some examples, the first step-up dose is administered to the subject during the first phase on Day 1 of C1 while the second step-up dose is administered on Day 6 of C1. In some examples, the first step-up dose is administered to the subject during the first phase on Day 1 of C1 while the second step-up dose is administered on Day 7 of C1.

If the subject experiences a CRS event following administration of a first step-up dose (e.g., 0.3 mg) during the first phase on Day 1 of C1, administration of the second dose (e.g., 3.3 mg) may be delivered on Day 2, Day 3, or Day 4, upon full resolution of CRS. Additional dose delay (e.g., delivery of the second dose on Day 5, Day 6, or Day 7) may be necessary depending on the clinical presentation of the CRS event. Refer to the CRS management guidelines set forth in Table 3A and Table 3B.

In a further example, a target dose is administered to the subject during the first phase following the administration of the second step-up dose. In some examples, the target dose is administered to the subject on Day 8 of C1. In some examples, the target dose is administered to the subject on Day 9 of C1. In some examples, the target dose is administered to the subject on or after Day 9 (e.g., Day 9, Day 10, Day 11, Day 12, Day 13, Day 14, Day 15, Day 16, Day 17, Day 18, Day 19, Day 20, or Day 21) of C1.

In a further example, the target dose is further administered to the subject during the first phase on Day 1 of C2. In a further example, the target dose is administered to the subject during the first phase on Day 1 of C3. In a further example, the target dose is administered to the subject during the first phase on Day 1 of C4. In a further example, the target dose is administered to the subject during the first phase on Day 1 of C5. In a further example, the target dose is administered to the subject during the first phase on Day 1 of C6. In a further example, the target dose is administered to the subject during the first phase on Day 1 of C7. In a further example, the target dose is administered to the subject during the first phase on Day 1 of C8. In a further example, the target dose is administered to the subject during the first phase on Day 1 of C9. In a further example, the target dose is administered to the subject during the first phase on Day 1 of C10. In a further example, the target dose is administered to the subject during the first phase on Day 1 of C11. In a further example, the target dose is administered to the subject during the first phase on Day 1 of C12. In a further example, the target dose is administered to the subject during the first phase on Day 1 of C13.

In some examples, the first step-up dose is about 0.1% to about 2% of the target dose and the second step-up dose is about 2% to about 8% of the target dose. In some examples, the first step-up dose is about 0.11%, about 0.12%, about 0.13%, about 0.14%, about 0.15%, about 0.16%, about 0.17%, about 0.18%, about 0.19%, about 0.2%, about 0.21%, about 0.22%, about 0.23%, about 0.24%, about 0.25%, about 0.26%, about 0.27%, about 0.28%, about 0.29%, about 0.3%, about 0.4%, about 0.5%, about 0.6%, about 0.7%, about 0.8%, about 0.9%, about 1%, about 1.5%, or about 2% of the target dose and the second step-up dose is about 2%, about 2.03%, about 2.1%, about 2.2%, about 2.3%, about 2.4%, about 2.5%, about 3%, about 3.5%, about 4%, about 4.5%, about 5%, about 5.5%, about 6%, about 6.5%, about 7%, about 7.5%, or about 8% of the target dose. In some examples, the first step-up dose is about 0.19% of the target dose and the second step-up dose is about 2.06% of the target dose. In some examples, the first step-up dose is about 0.19% of the target dose and the second step-up dose is about 2.3% of the target dose.

In some examples, the first step-up dose is 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.4%, 0.5%, 0.6%, 0.7%, 0.8%, 0.9%, 1%, 1.5%, or 2% of the target dose and the second step-up dose is 2%, 2.03%, 2.1%, 2.2%, 2.3%, 2.4%, 2.5%, 3%, 3.5%, 4%, 4.5%, 5%, 5.5%, 6%, 6.5%, 7%, 7.5%, or 8% of the target dose. In some examples, the first step-up dose is 0.19% of the target dose and the second step-up dose is 2.06% of the target dose. In some examples, the first step-up dose is about 0.19% of the target dose and the second step-up dose is about 2.3% of the target dose.

In some examples, the first step-up dose is about 0.3 mg and the second step-up dose is about 3.3 mg. In some examples, the first step-up dose is about 0.3 mg and the second step-up dose is about 3.6 mg. In some examples, the first step-up dose is about 0.1 mg, about 0.2 mg, about 0.3 mg, about 0.4 mg, about 0.5 mg, about 0.6 mg, about 0.7 mg, about 0.8 mg, about 0.9 mg, or about 1 mg while the second step-up dose is about 1.5 mg, about 2 mg, about 2.5 mg, about 3 mg, about 3.3 mg, about 3.5 mg, about 3.6 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, or about 10 mg.

In some examples, the first step-up dose is 0.3 mg and the second step-up dose is 3.3 mg. In some examples, the first step-up dose is 0.3 mg and the second step-up dose is 3.6 mg. In some examples, the first step-up dose is 0.1 mg, 0.2 mg, 0.3 mg, 0.4 mg, 0.5 mg, 0.6 mg, 0.7 mg, 0.8 mg, 0.9 mg, or 1 mg while the second step-up dose is 1.5 mg, 2 mg, 2.5 mg, 3 mg, 3.3 mg, 3.5 mg, 3.6 mg, 4 mg, 4.5 mg, 5 mg, 5.5 mg, 6 mg, 6.5 mg, 7 mg, 7.5 mg, 8 mg, 8.5 mg, 9 mg, 9.5 mg, or 10 mg.

In any of the foregoing examples, the second phase may comprise at least two dosing cycles, at least three dosing cycles, or at least four dosing cycles, at least five dosing cycles, at least six dosing cycles, at least seven, at least eight dosing cycle, at least nine dosing cycle, at least ten dosing cycle, at least eleven dosing cycles, at least twelve dosing cycles, or at least thirteen dosing cycles, or more. For example, the second phase may continue until the subject experiences disease progression, unacceptable toxicity, or death.

The second phase may comprise any suitable number of dosing cycles. For example, in some examples, the second phase may comprises a first dosing cycle (C1); a first dosing cycle and a second dosing cycle (C2); a first dosing cycle, a second dosing cycle (C2), and a third dosing cycle (C3); a first dosing cycle (C1), a second dosing cycle (C2), a third dosing cycle (C3), and a fourth dosing cycle (C4); a first dosing cycle (C1), a second dosing cycle (C2), a third dosing cycle (C3), a fourth dosing cycle (C4), and a fifth dosing cycle (C5); a first dosing cycle (C1), a second dosing cycle (C2), a third dosing cycle (C3), a fourth dosing cycle (C4), a fifth dosing cycle (C5), and a sixth dosing cycle (C6); a first dosing cycle (C1), a second dosing cycle (C2), a third dosing cycle (C3), a fourth dosing cycle (C4), a fifth dosing cycle (C5), a sixth dosing cycle (C6), and a seventh dosing cycle (C7); a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), and an eighth dosing cycle (C8); a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), and a ninth dosing cycle (C9); a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), and a tenth dosing cycle (C10); a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), and an eleventh dosing cycle (C11); a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), and a twelfth dosing cycle (C12); or a first dosing cycle (C1), a second dosing cycle (C2), a third dosing cycle (C3), a fourth dosing cycle (C4), a fifth dosing cycle (C5), a sixth dosing cycle (C6), a seventh dosing cycle (C7), an eighth dosing cycle (C8), a ninth dosing cycle (C9), a tenth dosing cycle (C10), an eleventh dosing cycle (C11), a twelfth dosing cycle (C12), and a thirteenth dosing cycle (C13).

In some examples, a target dose of the bispecific antibody may be administered to the subject during the second phase on Day 1 of C1. In a further example, the target dose of the bispecific antibody may be administered to the subject during the second phase on Day 1 of C2. In a further example, the target dose of the bispecific antibody may be administered to the subject during the second phase on Day 1 of C3. In a further example, the target dose of the bispecific antibody may be administered to the subject during the second phase on Day 1 of C4. In a further example, the target dose of the bispecific antibody may be administered to the subject during the second phase on Day 1 of C5. In a further example, the target dose of the bispecific antibody may be administered to the subject during the second phase on Day 1 of C6. In a further example, the target dose of the bispecific antibody may be administered to the subject during the second phase on Day 1 of C7. In a further example, the target dose of the bispecific antibody may be administered to the subject during the second phase on Day 1 of C8. In a further example, the target dose of the bispecific antibody may be administered to the subject during the second phase on Day 1 of C9. In a further example, the target dose of the bispecific antibody may be administered to the subject during the second phase on Day 1 of C10. In a further example, the target dose of the bispecific antibody may be administered to the subject during the second phase on Day 1 of C11. In a further example, the target dose of the bispecific antibody may be administered to the subject during the second phase on Day 1 of C12. In a further example, the target dose of the bispecific antibody may be administered to the subject during the second phase on Day 1 of C13. In any of the foregoing examples, the second phase comprises a C1, and Day 1 of the C1 of the second phase is at least 7 days after administration of a target dose of the bispecific antibody in the first phase.

In some examples, a target dose of the bispecific antibody is administered to the subject for each administration during the second phase.

In any of the foregoing examples, the target dose may be about 45 mg to about 180 mg. In some examples, the target dose is about 50 mg to about 175 mg. In some examples, the target dose is about 55 mg to about 165 mg. In some examples, the target dose is about 60 mg to about 160 mg. In some examples, the target dose is about 65 mg to about 155 mg. In some examples, the target dose is about 70 mg to about 150 mg. In some examples, the target dose is about 75 mg to about 145 mg. In some examples, the target dose is about 80 mg to about 140 mg. In some examples, the target dose is about 85 mg to about 135 mg. In some examples, the target dose is about 90 mg to about 130 mg. In some examples, the target dose is about 40 mg. In some examples, the target dose is about 90 mg. In some examples, the target dose is about 120 mg. In some examples, the target dose is about 132 mg. In some examples, the target dose is about 160 mg. In some examples, the target dose is about 198 mg. In some examples, the target dose is about 252 mg.

In some examples, the target dose is about 40 mg. In some examples, the target dose is about 45 mg. In some examples, the target dose is about 50 mg. In some examples, the target dose is about 55 mg. In some examples, the target dose is about 60 mg. In some examples, the target dose is about 65 mg. In some examples, the target dose is about 70 mg. In some examples, the target dose is about 75 mg. In some examples, the target dose is about 80 mg. In some examples, the target dose is about 85 mg. In some examples, the target dose is about 90 mg. In some examples, the target dose is about 95 mg. In some examples, the target dose is about 100 mg. In some examples, the target dose is about 105 mg. In some examples, the target dose is about 110 mg. In some examples, the target dose is about 115 mg. In some examples, the target dose is about 120 mg. In some examples, the target dose is about 125 mg. In some examples, the target dose is about 130 mg. In some examples, the target dose is about 132 mg. In some examples, the target dose is about 135 mg. In some examples, the target dose is about 140 mg. In some examples, the target dose is about 145 mg. In some examples, the target dose is about 150 mg. In some examples, the target dose is about 155 mg. In some examples, the target dose is about 160 mg. In some examples, the target dose is about 165 mg. In some examples, the target dose is about 170 mg. In some examples, the target dose is about 175 mg. In some examples, the target dose is about 180 mg. In some examples, the target dose is about 185 mg. In some examples, the target dose is about 189 mg. In some examples, the target dose is about 195 mg. In some examples, the target dose is about 198 mg. In some examples, the target dose is about 200 mg. In some examples, the target dose is about 205 mg. In some examples, the target dose is about 210 mg. In some examples, the target dose is about 215 mg. In some examples, the target dose is about 220 mg. In some examples, the target dose is about 225 mg. In some examples, the target dose is about 230 mg. In some examples, the target dose is about 235 mg. In some examples, the target dose is about 240 mg. In some examples, the target dose is about 245 mg. In some examples, the target dose is about 250 mg. In some examples, the target dose is about 252 mg. In some examples, the target dose is about 255 mg. In some examples, the target dose is about 260 mg.

In some examples, the target dose is 45 mg to 180 mg. In some examples, the target dose is 50 mg to 175 mg. In some examples, the target dose is 55 mg to 165 mg. In some examples, the target dose is 60 mg to 160 mg. In some examples, the target dose is 65 mg to 155 mg. In some examples, the target dose is 70 mg to 150 mg. In some examples, the target dose is 75 mg to 145 mg. In some examples, the target dose is 80 mg to 140 mg. In some examples, the target dose is 85 mg to 135 mg. In some examples, the target dose is 90 mg to 130 mg. In some examples, the target dose is 40 mg. In some examples, the target dose is 90 mg. In some examples, the target dose is 120 mg. In some examples, the target dose is 132 mg.

In some examples, the target dose is 160 mg. In some examples, the target dose is 198 mg. In some examples, the target dose is 252 mg.

In some examples, the target dose is 40 mg. In some examples, the target dose is 45 mg. In some examples, the target dose is 50 mg. In some examples, the target dose is 55 mg. In some examples, the target dose is 60 mg. In some examples, the target dose is 65 mg. In some examples, the target dose is 70 mg. In some examples, the target dose is 75 mg. In some examples, the target dose is 80 mg. In some examples, the target dose is 85 mg. In some examples, the target dose is 90 mg. In some examples, the target dose is 95 mg. In some examples, the target dose is 100 mg. In some examples, the target dose is 105 mg. In some examples, the target dose is 110 mg. In some examples, the target dose is 115 mg. In some examples, the target dose is 120 mg. In some examples, the target dose is 125 mg. In some examples, the target dose is 130 mg. In some examples, the target dose is 132 mg. In some examples, the target dose is 135 mg. In some examples, the target dose is 140 mg. In some examples, the target dose is 145 mg. In some examples, the target dose is 150 mg. In some examples, the target dose is 155 mg. In some examples, the target dose is 160 mg. In some examples, the target dose is 165 mg. In some examples, the target dose is 170 mg. In some examples, the target dose is 175 mg. In some examples, the target dose is 180 mg. In some examples, the target dose is 185 mg. In some examples, the target dose is 189 mg. In some examples, the target dose is 195 mg. In some examples, the target dose is 198 mg. In some examples, the target dose is 200 mg. In some examples, the target dose is 205 mg. In some examples, the target dose is 210 mg. In some examples, the target dose is 215 mg. In some examples, the target dose is 220 mg. In some examples, the target dose is 225 mg. In some examples, the target dose is 230 mg. In some examples, the target dose is 235 mg. In some examples, the target dose is 240 mg. In some examples, the target dose is 245 mg. In some examples, the target dose is 250 mg. In some examples, the target dose is 252 mg. In some examples, the target dose is 255 mg. In some examples, the target dose is 260 mg.

In some examples, the bispecific antibody is administered to the subject as a monotherapy.

In some examples, the bispecific antibody is administered to the subject intravenously. In some examples, the bispecific antibody is administered to the subject subcutaneously.

In any of the foregoing examples, the dosing regimen further comprises administration of a corticosteroid to the subject during the first phase and/or the second phase. For example, in some examples, the dosing regimen further comprises administration of a corticosteroid to the subject during the run-in phase.

In another example, the dosing regimen further comprises administration of a corticosteroid to the subject during the first phase. In another example, the dosing regimen further comprises administration of a corticosteroid to the subject during the second phase. In other examples, the dosing regimen further comprises administration of a corticosteroid to the subject during the first phase and the second phase.

In some examples, the dosing regimen described herein is for treating a subject having an R/R MM, wherein the subject is administered a bispecific antibody that binds to FcRH5 and CD3. In some examples, the bispecific antibody is administered in a dosing regimen containing at least a first 7-day dosing cycle. In some examples, the bispecific antibody is administered in a dosing regimen containing at least a first 14-day dosing cycle. In some examples, the bispecific antibody is administered in a dosing regimen containing at least a first 21-day dosing cycle. In some examples, the bispecific antibody is administered in a dosing regimen containing at least a first 28-day dosing cycle. In some examples, the dosing cycle (e.g., a 7-day, 14-day, 21-day, or 28-day dosing cycle), comprises a first dose (C1D1), a second dose (C1D2), and a third dose (C1D3). In some examples, the C1D1 is about 0.2 mg to about 0.4 mg (e.g., about 0.2 mg, about 0.3 mg, or about 0.4 mg), the C1D2 is about 3.1 mg to about 3.4 mg (e.g., about 3.1 mg, about 3.2 mg, about 3.3 mg, or about 3.4 mg), and the C1D3 is an amount that is greater than the C1D2. In some examples, the C1D1 is 0.2 mg to 0.4 mg (e.g., 0.2 mg, 0.3 mg, or about 0.4 mg), the C1D2 is 3.1 mg to 3.4 mg (e.g., 3.1 mg, 3.2 mg, 3.3 mg, or 3.4 mg), and the C1D3 is an amount that is greater than the C1D2.

In some examples, the dosing regimen described herein is for treating a subject having a triple-class refractory MM, wherein the subject is administered a cevostamab monotherapy. In some examples, the subject has previously received a BCMA-targeting TDB antibody. In some examples, the subject has previously received a BCMA-targeting CAR-T. In some examples, the subject has previously received a BCMA-targeting ADC. In some examples, the cevostamab monotherapy is administered in a dosing regimen containing a first phase and a second phase. In some examples, the cevostamab is administered to the subject in a first dosing cycle (C1) during the first phase. In some examples, the cevostamab is administered to the subject Q3W during the second phase until the subject experiences disease progression, unacceptable toxicity, or death. In some examples, each dosing cycle of the first phase and second phase is a 21-day dosing cycle. In some examples, the cevostamab is administered to the subject at a first step-up dose of 0.3 mg during the first phase on Day 1 of the C1 and as a second step-up dose of 3.3 mg during the first phase on Day 2 of the C1. In other examples the cevostamab is administered to the subject at a first step-up dose of 0.3 mg during the first phase on Day 1 of the C1 and as a second step-up dose of 3.6 mg during the first phase on Day 2 of the C1. In other examples, the cevostamab is administered to the subject at a first step-up dose of 0.3 mg during the first phase on Day 1 of the C1 and as a second step-up dose of 3.3 mg during the first phase on Day 3 of the C1. In other examples, the cevostamab is administered to the subject at a first step-up dose of 0.3 mg during the first phase on Day 1 of the C1 and as a second step-up dose of 3.6 mg during the first phase on Day 3 of the C1. In other examples, the cevostamab is administered to the subject at a first step-up dose of 0.3 mg during the first phase on Day 1 of the C1 and as a second step-up dose of 3.3 mg during the first phase on Day 4 of the C1. In other examples, the cevostamab is administered to the subject at a first step-up dose of 0.3 mg during the first phase on Day 1 of the C1 and as a second step-up dose of 3.6 mg during the first phase on Day 4 of the C1. In any of the foregoing examples, the cevostamab is administered at a target dose of 160 mg during the first phase on Day 8 or Day 9 of the C1 and during the second phase on Day 1 of each dosing cycle.

In some examples, the disclosure provides a bispecific antibody that binds to FcRH5 and CD3 for use in treatment of a subject having a R/R MM, wherein the subject has a triple class refractory MM and has previously received a BCMA-targeting therapeutic agent. In some examples, the treatment contains an administration of the bispecific antibody to the subject in a dosing regimen containing a first phase and a second phase. In some examples, the first phase contains a first 21-day dosing cycle (C1), wherein administration of the bispecific antibody to the subject occurs on Day 1, Day 2, and Day 8 of the C1. In some examples, the first phase contains a first 21-day dosing cycle (C1), wherein administration of the bispecific antibody to the subject occurs on Day 1, Day 3, and Day 8 of the C1. In some examples, the first phase contains a first 21-day dosing cycle (C1), wherein administration of the bispecific antibody to the subject occurs on Day 1, Day 4, and Day 8 of the C1. In some examples, the first phase contains a first 21-day dosing cycle (C1), wherein administration of the bispecific antibody to the subject occurs on Day 1, Day 2, and Day 9 of the C1. In some examples, the first phase contains a first 21-day dosing cycle (C1), wherein administration of the bispecific antibody to the subject occurs on Day 1, Day 3, and Day 9 of the C1. In some examples, the first phase contains a first 21-day dosing cycle (C1), wherein administration of the bispecific antibody to the subject occurs on Day 1, Day 4, and Day 9 of the C1. In some examples, the second phase contains one or more 21-day dosing cycles, wherein administration of the bispecific antibody to the subject occurs Q3W.

In some examples, the disclosure provides a bispecific antibody that binds to FcRH5 and CD3 for use in treatment of a subject having an R/R MM. In some examples, the subject is administered the bispecific antibody that binds to FcRH5 and CD3 in a dosing regimen containing at least a first 21-day dosing cycle, wherein the first dosing cycle contains a first dose (C1D1), a second dose (C1D2), and a third dose (C1D3) of the bispecific antibody. In some examples, the C1D1 is between about 0.2 mg to about 0.4 mg (e.g., 0.1 mg, 0.2 mg, 0.3 mg, 0.4 mg, or 0.5 mg) and is administered to the subject on Day 1 of the first dosing cycle. In some examples, the C1D2 is about 3.1 mg to about 3.4 mg (e.g., 3.0 mg, 3.1 mg, 3.2 mg, 3.3 mg, 3.4 mg, or 3.5 mg) and is administered to the subject on Day 2 of the first dosing cycle. In some examples, the C1D3 is greater than the C1D2.

### C. Combination Therapies

In some instances, the bispecific anti-FcRH5/anti-CD3 antibody is administered to the subject in a combination therapy. For example, the bispecific anti-FcRH5/anti-CD3 antibody may be co-administered with one or more additional therapeutic agents described herein.

i. Anti-CD38 Antibodies

In some instances, the bispecific anti-FcRH5/anti-CD3 antibody is administered to the subject in combination with an anti-CD38 antibody. The anti-CD38 antibody may be administered by any suitable administration route, e.g., intravenously (IV) or subcutaneously (SC) to the subject. In some aspects, the anti-CD38 antibody is daratumumab (e.g., daratumumab/rHuPH20). The daratumumab may be administered to the subject at a dose of about 900 mg to about 3600 mg (e.g., about 900 mg, about 950 mg, about 1000 mg, about 1100 mg, about 1200 mg, about 1300 mg, about 1400 mg, about 1500 mg, about 1600 mg, about 1650 mg, about 1700 mg, about 1750 mg, about 1800 mg, about 1850 mg about 1900 mg, about 1950 mg, about 2000 mg, about 2100 mg, about 2200 mg, about 2300 mg, about 2400 mg, about 2500 mg, about 2600 mg, about 2700 mg, about 2800 mg, about 2900 mg, about 3000 mg, about 3100 mg, about 3200 mg, about 3300 mg, about 3400 mg, about 3500 mg, or about 3600 mg). The daratumumab may be administered to the subject at a dose of about 1800 mg. In some aspects, the daratumumab is administered by intravenous infusion (e.g., infusion over 3-5 hours) at a dose of 16 mg/kg once every week, once every two weeks, or once every four weeks. In some aspects, the daratumumab is administered by intravenous infusion (e.g., infusion over 3-5 hours) at a dose of 16 mg/kg. In other aspects, the anti-CD38 antibody is isatuximab. In some aspects, the anti-CD38 antibody (e.g., daratumumab or isatuxamab) is administered to the subject prior to the administration of the bispecific anti-FcRH5/anti-CD3 antibody, e.g., administered one day prior to the administration of the bispecific anti-FcRH5/anti-CD3 antibody. In some aspects, the anti-CD38 antibody (e.g., daratumumab or isatuxamab) is administered to the subject concurrently with the administration of the bispecific anti-FcRH5/anti-CD3 antibody.

ii. Corticosteroids

In some instances, the bispecific anti-FcRH5/anti-CD3 antibody is administered to the subject in combination with a corticosteroid. The corticosteroid may be administered orally to the subject. The corticosteroid may be administered by any suitable administration route, e.g., intravenously to the subject. Any suitable corticosteroid may be used, e.g., dexamethasone, methylprednisolone, prednisone, prednisolone, betamethasone, hydrocortisone, and the like. In some aspects, the corticosteroid is methylprednisolone. The methylprednisolone may be administered to the subject at a dose of about 80 mg. In other aspects, the corticosteroid is dexamethasone. The dexamethasone may be administered to the subject at a dose of about 20 mg. In some aspects, the corticosteroid (e.g., methylprednisolone or dexamethasone) is administered to the subject prior to the administration of the bispecific anti-FcRH5/anti-CD3 antibody, e.g., administered one hour prior to the administration of the bispecific anti-FcRH5/anti-CD3 antibody. In some aspects, the corticosteroid (e.g., methylprednisolone or dexamethasone) is administered to the subject about one day prior to the administration of the bispecific anti-FcRH5/anti-CD3 antibody. In some aspects, the corticosteroid (e.g., methylprednisolone or dexamethasone) is administered to the subject concurrently with the administration of the bispecific anti-FcRH5/anti-CD3 antibody.

The corticosteroid may be administered by any suitable administration route. In some examples, the corticosteroid is administered to the subject intravenously. In some examples, the corticosteroid is administered to the subject orally. In some examples, the corticosteroid may be administered to the subject intravenously or orally.

In some examples, the corticosteroid is administered to the subject intravenously prior to the administration of the bispecific antibody. In some examples, the corticosteroid is administered to the subject intravenously about 1 hour prior to the administration of the bispecific antibody.

In some examples, the corticosteroid is dexamethasone or methylprednisolone. In some examples, the corticosteroid is dexamethasone.

In some examples, dexamethasone is administered to the subject at a dosage of about 10 mg to about 40 mg. In some examples, dexamethasone is administered to the subject at a dosage of about 10 mg, about 15 mg, about 20 mg, about 25 mg, about 30 mg, about 35 mg, or about 40 mg. In some examples, dexamethasone is administered to the subject at a dosage of about 20 mg.

In some examples, dexamethasone is administered to the subject at a dosage of 10 mg to 40 mg. In some examples, dexamethasone is administered to the subject at a dosage of 10 mg, 15 mg, 20 mg, 25 mg, 30 mg, 35 mg, or 40 mg. In some examples, dexamethasone is administered to the subject at a dosage of 20 mg.

In some examples, methylprednisolone is administered to the subject at a dosage of about 40 mg to about 160 mg. In some examples, methylprednisolone is administered to the subject at a dosage of about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, or about 160 mg. In some examples, methylprednisolone is administered to the subject at a dosage of about 80 mg.

In some examples, methylprednisolone is administered to the subject at a dosage of 40 mg to 160 mg. In some examples, methylprednisolone is administered to the subject at a dosage of 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, or 160 mg. In some examples, methylprednisolone is administered to the subject at a dosage of 80 mg.

iii. Immunomodulatory Drugs (IMiD)

In some instances, the bispecific anti-FcRH5/anti-CD3 antibody is administered to the subject in combination with an immunomodulatory drug (IMiD). The IMiD may be administered by any suitable administration route, e.g., orally to the subject. The IMiD may be administered intravenously to the subject. In some aspects, the IMiD is pomalidomide. The pomalidomide may be administered to the subject at a dose of about 4 mg. In other aspects, the IMiD is lenalidomide. In some aspects, the IMiD (e.g., pomalidomide or lenalidomide) is administered to the subject prior to the administration of the bispecific anti-FcRH5/anti-CD3 antibody, e.g., administered one hour prior to the administration of the bispecific anti-FcRH5/anti-CD3 antibody. In some aspects, the IMiD (e.g., pomalidomide or lenalidomide) is administered to the subject concurrently with the administration of the bispecific anti-FcRH5/anti-CD3 antibody. In some aspects, the IMiD (e.g., pomalidomide or lenalidomide) is administered daily between doses of the bispecific anti-FcRH5/anti-CD3 antibody.

iv. Tocilizumab and Treatment of CRS

In one instance, the additional therapeutic agent is an effective amount of tocilizumab (ACTEMRA®). In some instances, the subject has a cytokine release syndrome (CRS) event (e.g., has a CRS event following treatment with the bispecific antibody, e.g., has a CRS event following a C1D1, a C1D2, a C1D3, a C2D1, or an additional dose of the bispecific antibody), and the method further comprises treating the symptoms of the CRS event (e.g., treating the CRS event by administering to the subject an effective amount of tocilizumab) while suspending treatment with the bispecific antibody. In some aspects, tocilizumab is administered intravenously to the subject as a single dose of about 8 mg/kg. In some aspects, the CRS event does not resolve or worsens within 24 hours of treating the symptoms of the CRS event, and the method further comprising administering to the subject one or more additional doses of tocilizumab to manage the CRS event, e.g., administering one or more additional doses of tocilizumab intravenously to the subject at a dose of about 8 mg/kg.

In some aspects, treating the symptoms of the CRS event further comprises treatment with a high-dose vasopressor (e.g., norepinephrine, dopamine, phenylephrine, epinephrine, or vasopressin and norepinephrine), e.g., as described in Tables 2A, 2B, and 8.

In other instances, tocilizumab is administered as a premedication, e.g., is administered to the subject prior to the administration of the bispecific anti-FcRH5/anti-CD3 antibody. In some instances, tocilizumab is administered as a premedication in Cycle 1, e.g., is administered prior to a first dose (C1D1) of the bispecific antibody, a second dose (C1D2) of the bispecific antibody, and/or a third dose (C1D3) of the bispecific anti-FcRH5/anti-CD3 antibody. In some aspects, the tocilizumab is administered intravenously to the subject as a single dose of about 8 mg/kg.

v. CRS Symptoms and Grading

CRS may be graded according to the Modified Cytokine Release Syndrome Grading System established by Lee et al., Blood, 124: 188-195, 2014, Biol Blood Marrow Transplant, 25(4): 625-638, 2019, as described in Table 2A. In addition to diagnostic criteria, recommendations on management of CRS based on its severity, including early intervention with corticosteroids and/or anti-cytokine therapy, are provided and referenced in Tables 2A and 2B.

TABLE 2A
Cytokine release syndrome grading systems

| | Modified Cytokine Release | ASTCT Consensus |
|---|---|---|
| Grade | Syndrome Grading System | Grading System |
| Grade 1 | Symptoms are not life threatening and | Temperature ≥38° C. |
| | require symptomatic treatment only (e.g., | No hypotension |
| | fever, nausea, fatigue, headache, myalgia, | No hypoxia |
| | malaise) | |
| Grade 2 | Symptoms require and respond to | Temperature ≥38° C.* with |
| | moderate intervention | hypotension not requiring |
| | Oxygen requirement <40%; or | vasopressors and/or^{†} hypoxia |
| | Hypotension responsive to fluids or low | requiring low-flow nasal cannula^{‡} or |
| | dose ^{a} of one vasopressor; or | blow-by |
| | Grade 2 organ toxicity | |
| Grade 3 | Symptoms require and respond to | Temperature ≥38° C.* with |
| | aggressive intervention | hypotension requiring a vasopressor |
| | Oxygen requirement ≥40%; or | with or without vasopressin and/or^{†} |
| | Hypotension requiring high dose ^{b} or | hypoxia requiring high-flow nasal |
| | multiple vasopressors; or | |
| | Grade 3 organ toxicity or Grade 4 | cannula^{‡}, facemask, nonrebreather |
| | transaminitis | mask, or Venturi mask |
| Grade 4 | Life-threatening symptoms | Temperature ≥38° C.* with |
| | Requirement for ventilation support or | hypotension requiring multiple |
| | Grade 4 organ toxicity (excluding | vasopressors (excluding vasopressin) |
| | transaminitis) | and/ort hypoxia requiring positive |
| | | pressure (e.g., CPAP, BiPAP, |
| | | intubation and mechanical ventilation) |
| Grade 5 | Death | Death |

TABLE 2B
High-dose vasopressors
High-Dose Vasopressors (duration ≥3 hours)

| Pressor | Dose | |
|---|---|---|
| Norepinephrine monotherapy | ≥20 | μg/min |
| Dopamine monotherapy | ≥10 | μg/kg/min |
| Phenylephrine monotherapy | ≥200 | μg/min |
| Epinephrine monotherapy | ≥10 | μg/min |

| | |
|---|---|
| If on vasopressin | Vasopressin + norepinephrine equivalent of ≥10 μg/min ^{a} |
| If on combination or vasopressors | Norepinephrine equivalent of ≥20 μg/min ^{a} |
| (not vasopressin) | |

Mild to moderate presentations of CRS and/or infusion-related reaction (IRR) may include symptoms such as fever, headache, and myalgia, and may be treated symptomatically with analgesics, anti-pyretics, and antihistamines as indicated. Severe or life-threatening presentations of CRS and/or IRR, such as hypotension, tachycardia, dyspnea, or chest discomfort should be treated aggressively with supportive and resuscitative measures as indicated, including the use of high-dose corticosteroids, IV fluids, admission to intensive care unit, and other supportive measures. Severe CRS may be associated with other clinical sequelae such as disseminated intravascular coagulation, capillary leak syndrome, or macrophage activation syndrome (MAS). Standard of care for severe or life-threatening CRS resulting from immune-based therapy has not been established; case reports and recommendations using anti-cytokine therapy such as tocilizumab have been published (Teachey et al., Blood, 121: 5154-5157, 2013; Lee et al., Blood, 124: 188-195, 2014; Maude et al., New Engl J Med, 371: 1507-1517, 2014).

As noted in Table 2A, even moderate presentations of CRS in subjects with extensive comorbidities should be monitored closely, with consideration given to intensive care unit admission and tocilizumab administration.

vi. Administration of Tocilizumab as a Premedication

In some aspects, an effective amount of an interleukin-6 receptor (IL-6R) antagonist (e.g., an anti-IL-6R antibody, e.g., tocilizumab (ACTEMRA®/ROACTEMRA®)) is administered as a premedication (prophylaxis), e.g., is administered to the subject prior to the administration of the bispecific antibody (e.g., administered about 2 hours prior to the administration of the bispecific antibody). Administration of tocilizumab as a premedication may reduce the frequency or severity of CRS. In some aspects, tocilizumab is administered as a premedication in Cycle 1, e.g., is administered prior to a first dose (C1D1; cycle 1, dose 1), a second dose (C1D2; cycle 1, dose 2), and/or a third dose (C1D3; cycle 1, dose 3) of the bispecific antibody. In some aspects, the tocilizumab is administered intravenously to the subject as a single dose of about 1 mg/kg to about 15 mg/kg, e.g., about 4 mg/kg to about 10 mg/kg, e.g., about 6 mg/kg to about 10 mg/kg, e.g., about 8 mg/kg. In some aspects, the tocilizumab is administered intravenously to the subject as a single dose of about 8 mg/kg. In some aspects, the tocilizumab is administered intravenously to the subject as a single dose of about 8 mg/kg for patients weighing 30 kg or more (maximum 800 mg) and at a dose of about 12 mg/kg for patients weighing less than 30 kg. Other anti-IL-6R antibodies that could be used in combination with tocilizumab include sarilumab, vobarilizumab (ALX-0061), SA-237, and variants thereof.

For example, in one aspect, the bispecific antibody is co-administered with tocilizumab (ACTEMRA®/ROACTEMRA®), wherein the subject is first administered with tocilizumab (ACTEMRA®/ROACTEMRA®) and then separately administered with the bispecific antibody (e.g., the subject is pre-treated with tocilizumab (ACTEMRA®/ROACTEMRA®)).

In some aspects, the incidence of CRS (e.g., Grade 1 CRS, Grade 2 CRS, and/or Grade 3+ CRS) is reduced in patients who are treated with tocilizumab as a premedication relative to patients who are not treated with tocilizumab as a premedication. In some aspects, less intervention to treat CRS (e.g., less need for additional tocilizumab, IV fluids, steroids, or O₂) is required in patients who are treated with tocilizumab as a premedication relative to patients who are not treated with tocilizumab as a premedication. In some aspects, CRS symptoms have decreased severity (e.g., are limited to fevers and rigors) in patients who are treated with tocilizumab as a premedication relative to patients who are not treated with tocilizumab as a premedication.

vii. Tocilizumab Administered to Treat CRS

In some aspects, the subject experiences a CRS event during treatment with the therapeutic bispecific antibody and an effective amount of an IL-6R antagonist (e.g., an anti-IL-6R antibody, e.g., tocilizumab (ACTEMRA®/ROACTEMRA®)) is administered to manage the CRS event.

In some aspects, the subject has a CRS event (e.g., has a CRS event following treatment with the bispecific antibody, e.g., has a CRS event following a first dose or a subsequent dose of the bispecific antibody), and the method further includes treating the symptoms of the CRS event while suspending treatment with the bispecific antibody.

In some aspects, the subject experiences a CRS event, and the method further includes administering to the subject an effective amount of an interleukin-6 receptor (IL-6R) antagonist (e.g., an anti-IL-6R antibody, e.g., tocilizumab (ACTEMRA®/ROACTEMRA®)) to manage the CRS event while suspending treatment with the bispecific antibody. In some aspects, the IL-6R antagonist (e.g., tocilizumab) is administered intravenously to the subject as a single dose of about 1 mg/kg to about 15 mg/kg, e.g., about 4 mg/kg to about 10 mg/kg, e.g., about 6 mg/kg to about 10 mg/kg, e.g., about 8 mg/kg. In some aspects, the tocilizumab is administered intravenously to the subject as a single dose of about 8 mg/kg. Other anti-IL-6R antibodies that could be used in combination with tocilizumab include sarilumab, vobarilizumab (ALX-0061), SA-237, and variants thereof.

In some aspects, the CRS event does not resolve or worsens within 24 hours of treating the symptoms of the CRS event, and the method further includes administering to the subject one or more additional doses of the IL-6R antagonist (e.g., an anti-IL-6R antibody, e.g., tocilizumab) to manage the CRS event, e.g., administering one or more additional doses of tocilizumab intravenously to the subject at a dose of about 1 mg/kg to about 15 mg/kg, e.g., about 4 mg/kg to about 10 mg/kg, e.g., about 6 mg/kg to about 10 mg/kg, e.g., about 8 mg/kg. In some aspects, the one or more additional doses of tocilizumab are administered intravenously to the subject as a single dose of about 8 mg/kg.

In some aspects, the method further includes administering to the subject an effective amount of a corticosteroid. The corticosteroid may be administered intravenously to the subject. In some aspects, the corticosteroid is methylprednisolone. In some instances, the methylprednisolone is administered at a dose of about 1 mg/kg per day to about 5 mg/kg per day, e.g., about 2 mg/kg per day. In some instances, the corticosteroid is dexamethasone. In some instances, the dexamethasone is administered at a dose of about 10 mg (e.g., a single dose of about 10 mg intravenously) or at a dose of about 0.5 mg/kg/day.

The subject may be administered a corticosteroid, such as methylprednisolone or dexamethasone, if the CRS event is not managed with administration of the IL-6R antagonist (e.g., tocilizumab) alone. In some aspects, treating the symptoms of the CRS event further includes treatment with a high-dose vasopressor (e.g., norepinephrine, dopamine, phenylephrine, epinephrine, or vasopressin and norepinephrine), e.g., as described in Table 2A, Table 2B, and Table 7. Tables 3A and 2A provide details about tocilizumab treatment of severe or life-threatening CRS.

viii. Management of CRS Events by Grade

Management of the CRS events may be tailored based on the grade of the CRS (Tables 2A and 3A) and the presence of comorbidities. Table 3A provides recommendations for the management of CRS syndromes by grade. Table 3B provides recommendations for the management of IRR syndromes by grade.

TABLE 3A
Recommendations for management of cytokine release syndrome (CRS)

| Event ^{a, b} | Action to Be Taken ^{b} |
|---|---|
| Grade 1 | Immediate actions: |
| Fever (≥38° C. or 100.4° F. not | If cevostamab infusion is still ongoing, interrupt infusion |
| attributable to any other cause) | immediately. |
| | Call to ensure tocilizumab is readily available if needed. |
| | Provide supportive care for constitutional symptoms. ^{c} |
| | Treat fever and neutropenia if present. |
| | Monitor fluid balance; administer IV fluids as clinically indicated. |
| | Consider hospitalization until symptoms completely resolve. |
| | In case of rapid decline in overall health or prolonged CRS |
| | (>24 hours) or in patients with significant symptoms and/or |
| | comorbidities (per investigator discretion, e.g., impaired |
| | cardiovascular function, reduced pulmonary reserve), consider |
| | administration of IV corticosteroids and tocilizumab or |
| | another anti-cytokine agent based on characterization of |
| | CRS. |
| | Restarting infusion: |
| | If cevostamab infusion was interrupted, wait until 30 minutes after |
| | the event has resolved before restarting the infusion at 50% of the |
| | original infusion rate. |
| | If symptoms recur, discontinue infusion for this dose. |
| | Next dose: |
| | Pretreat with antihistamines, antipyretics, and/or analgesics. |
| | Pretreat with IV corticosteroids (dexamethasone 20 mg |
| | (preferred) or methylprednisolone 80 mg). |
| | Consider hospitalization for next dose. |
| | Consider extending infusion time (slower infusion rate) for |
| | subsequent cycles. |
| Grade 2 | Immediate actions: |
| Fever (≥38° C. or 100.4° F. not | Follow all Grade 1 recommendations. |
| attributable to any other cause) | Hold further cevostamab treatment until symptoms completely |
| Hypotension: responds to fluids, not | resolved. |
| requiring vasopressors | Hospitalize patient until complete resolution of signs and |
| Hypoxia: requiring low flow nasal | symptoms. |
| cannula or blow-by | Administer tocilizumab 8 mg/kg IV ^{d} |
| | Consider treatment with IV corticosteroids (such as |
| | methylprednisolone 2 mg/kg/day or, if neurologic symptoms are |
| | present, dexamethasone 10 mg IV every 6 hours if needed). ^{b} |
| | Monitor cardiac and other organ function closely. |
| | Manage constitutional symptoms and organ toxicities as |
| | required. |
| | Provide hemodynamic support with IV fluid bolus 250-500 mL up |
| | to 2000 mL total (avoid fluid overload). |
| | Provide oxygen for hypoxia. |
| | Admit to ICU as appropriate. |
| | If no improvement within 8-12 hours after administration of |
| | tocilizumab, manage as a Grade 3 event: |
| | Notify Medical Monitor if the patient does not improve by 24 |
| | hours. |
| | Initiate workup and assess for signs and symptoms of |
| | macrophage activation syndrome (MAS)/hemophagocytic |
| | lymphohistiocytosis (HLH). |
| | Restarting infusion: |
| | Wait until 30 minutes after the event has resolved before |
| | restarting the infusion at up to 25% of the original infusion rate. |
| | If symptoms recur, stop infusion immediately. Cevostamab |
| | should not be restarted. |
| | If hypotension or hypoxia recurs, manage as a Grade 3 event. |
| | Next cycle: |
| | May receive the next dose of cevostamab if symptoms resolve to |
| | Grade ≤1 for 3 consecutive days as follows: |
| | Consider hospitalization for at least 24 hours after the |
| | infusion. |
| | Administer cevostamab at 50% of the initial infusion rate of |
| | the previous cycle if the event occurred during or within 24 |
| | hours of the infusion. ^{e} |
| | Pretreat with antihistamines, antipyretics, and/or |
| | analgesics. |
| | Pretreat with IV corticosteroids (dexamethasone 20 mg |
| | (preferred) or methylprednisolone 80 mg). |
| | Subsequent cycles: |
| | If there is an occurrence of IRR or CRS Grade ≥3 in any of the |
| | subsequent cycles, permanently discontinue cevostamab |
| | regardless of recovery (see Grade 3 management guidelines). |
| | If there is an occurrence of a Grade ≤2 CRS in subsequent |
| | cycles, manage as indicated by severity (see Grade 1 or 2 |
| | management guidelines). |
| Grade 3 | Immediate actions: |
| Fever (≥38° C. or 100.4° F. not | Stop further infusion of cevostamab. |
| attributable to any other cause) | Provide supportive care and treat symptomatically as clinically |
| Hypotension: requires a | indicated. ^{c} |
| vasopressor with or without | Monitor fluid balance; administer IV fluids bolus 250-500 mL up to |
| vasopressin | 2000 mL total (avoid fluid overload); provide vasopressor support |
| Hypoxia: requiring a high-flow nasal | for hypotension with high and repeated doses if required. |
| cannula, face mask, non- rebreather | Hospitalize patient until complete resolution of signs and |
| mask, or Venturi mask | symptoms. |
| | Treat with IV corticosteroids (such as methylprednisolone 2 |
| | mg/kg/day or, if neurologic symptoms are present, |
| | dexamethasone 10 mg IV every 6 hours if needed). ^{b} |
| | Administer tocilizumab 8 mg/kg IV. ^{d} |
| | If there is no improvement after 8 hours, repeat tocilizumab |
| | administration. |
| | If no improvement within 8-12 hours after administering |
| | second dose of tocilizumab, manage as a Grade 4 event. |
| | Initiate workup and assess for signs and symptoms of |
| | MAS/HLH. |
| | Monitoring of cardiopulmonary and organ function in ICU is |
| | recommended. |
| | Provide oxygen for hypoxia. |
| | Notify Medical Monitor. |
| | Restarting infusion: |
| | Do not restart infusion of cevostamab. |
| | Next cycle: |
| | If the patient had a Grade ≥2 IRR or CRS in any previous not |
| | cycle, permanently discontinue cevostamab. |
| | Patients who experience Grade 3 wheezing, |
| | bronchospasm, or generalized urticaria must be |
| | discontinued from cevostamab. |
| | If the patient recovers to Grade ≤1 for 3 consecutive days, |
| | cevostamab can be administered in next cycle as follows: |
| | Hospitalize patient for at least 24 hours after the mask |
| | infusion. |
| | Administer cevostamab at 50% of the initial infusion rate of |
| | the previous cycle if the event occurred during or within 24 |
| | hours of the infusion. ^{e} |
| | Pretreat with antihistamines, antipyretics, and/or analgesics |
| | Pretreat with IV corticosteroids (dexamethasone 20 mg |
| | (preferred) or methylprednisolone 80 mg). |
| | If the patient experienced Grade 3 CRS following the Cycle 1 |
| | Day 1 dose, the step-up dose must be repeated. |
| | Subsequent cycles: |
| | If a Grade ≥3 CRS recurs, permanently discontinue cevostamab. |
| | If there is an occurrence of a Grade ≤2 CRS in subsequent |
| | cycles, manage as indicated by severity (i.e., Grade 1 or 2 |
| | management guidelines). |
| Grade 4 | Stop cevostamab infusion. |
| Fever (≥38° C. or 100.4° F. not | Follow all Grade 3 management guidelines. |
| attributable to any other cause) | Patient requires ICU admission for hemodynamic monitoring, |
| Hypotension: requires multiple | and/or mechanical ventilation, and/or IV fluids and vasopressors |
| vasopressors | as needed. |
| Hypoxia: requiring positive pressure | Administer tocilizumab 8 mg/kg IV (see footnote). ^{d} |
| (e.g., CPAP, BiPAP, intubation, and | Treat with IV corticosteroids (such as methylprednisolone 2 |
| mechanical ventilation) | mg/kg/day or, if neurologic symptoms are present, |
| | dexamethasone 10 mg IV every 6 hours if needed) |
| | For patients who are refractory to tocilizumab therapy, |
| | experimental therapies (e.g., siltuximab, anakinra, emapalumab, |
| | or LCK-inhibitors) ^{b} may be considered based on the discretion |
| | of the investigator and institutional guidelines. |
| | Permanently discontinue study treatment. |

BiPAP = bilevel positive airway pressure; CPAP = continuous positive airway pressure; CRS = cytokine release syndrome; HLH = hemophagocytic lymphohistiocytosis; ICU = intensive care unit; IV = intravenous; MAS = macrophage activation syndrome.
^{a} Refer to Table 2A for the complete description of grading of symptoms.
^{b} Guidance for CRS management based on Lee et al., Biol Blood, 25(4): 625-638, 2019 and Riegler et al. (2019).
^{c} Patients should be treated with acetaminophen and an antihistamine (e.g., diphenhydramine) if they have not been administered in the previous 4 hours. For bronchospasm, urticaria, or dyspnea, treat per institutional practice. Treat fever and neutropenia as required; consider broad-spectrum antibiotics and/or G-CSF if indicated.
^{d} Tocilizumab should be administered at dose of 8 mg/kg IV (8 mg/kg for patients ≥30 kg weight only; 12 mg/kg for patients <30 kg weight; doses exceeding 800 mg per infusion are not recommended); repeat every 8 hours as necessary (up to a maximum of 4 doses).
^{e} If the patient does not experience CRS during the next infusion at the 50% reduced rate, the infusion rate can be increased to the initial rate in subsequent cycles. However, if this patient experiences another CRS event, the infusion rate should be reduced by 25%-50% depending on the severity of the event.

TABLE 3B
Recommendations for management of cevostamab infusion related reactions (IRR)

| Event ^{a} | Action to Be Taken | |
|---|---|---|
| Grade 1-2 | Slow infusion to ≤50% or interrupt infusion. | |
| | Give supportive treatment. ^{b} | |
| | Upon symptom resolution, may resume infusion (if interrupted) at 50% | |
| | starting rate. The infusion must remain at the lower rate, resulting in | |
| | symptom resolution for the remainder of the infusion. ^{c} | |
| | Premedicate with acetaminophen/paracetamol and an antihistamine such as | |
| | diphenhydramine for all subsequent infusions. | |
| | Notes: | |
| | 1. | For Grade 2 wheezing or urticaria, patient must be premedicated prior to |
| | | subsequent doses. |
| | 2. | If symptoms recur with the same or greater severity following the slower |
| | | or interrupted infusion of cevostamab, the infusion must be stopped |
| | | immediately. No further cevostamab should be administered for the |
| | | cycle. |
| Grade 3 | Discontinue cevostamab infusion. Do not re-initiate infusion for the | |
| | current cycle. | |
| | Give supportive treatment. ^{b} | |
| | Admission to ICU is recommended to monitor cardiopulmonary and other | |
| | organ functions. | |
| | Provide oxygen for hypoxia and/or mechanical ventilation as needed. | |
| | Subsequent cycles of cevostamab may be administered with | |
| | premedication. | |
| | Patients who experience Grade 3 wheezing, bronchospasm, or generalized | |
| | urticaria at first occurrence must be discontinued from study treatment. | |
| | Notes: | |
| | 1. | If symptoms recur despite premedications with the same or greater |
| | | severity at subsequent cycles, the infusion must be stopped immediately |
| | | and patient permanently discontinued from study treatment. |

| | |
|---|---|
| Grade 4 | Discontinue infusion immediately. |
| | Give supportive treatment. ^{b} |
| | Admit to ICU to monitor cardiopulmonary and other organ functions. |
| | Provide oxygen for hypoxia and/or mechanical ventilation as needed. |
| | Permanently discontinue study treatment. |

ICU = intensive care unit; NCI CTCAE = National Cancer Institute Common Terminology Criteria for Adverse Events.
^{a} Refer to NCI CTCAE v5.0 for the grading of symptoms.
^{b} Supportive treatment: Patients should be treated with acetaminophen/paracetamol and an antihistamine such as diphenhydramine if they have not been administered in the last 4 hours. Intravenous fluids (e.g., normal saline) may be administered as clinically indicated. For bronchospasm, urticaria, or dyspnea, antihistamines, oxygen, corticosteroids (e.g., 100 mg IV prednisolone or equivalent), and/or bronchodilators may be administered per institutional practice. Provide fluids and vasopressor support for hypotension if required.
^{c} Subsequent infusions of cevostamab may be started at the original rate.

ix. Management of Grade 2 CRS Events

If the subject has a grade 2 CRS event (e.g., a grade 2 CRS event in the absence of comorbidities or in the presence of minimal comorbidities) following administration of the therapeutic bispecific antibody, the method may further include treating the symptoms of the grade 2 CRS event while suspending treatment with the bispecific antibody. If the grade 2 CRS event then resolves to a grade ≤1 CRS event for at least three consecutive days, the method may further include resuming treatment with the bispecific antibody without altering the dose. On the other hand, if the grade 2 CRS event does not resolve or worsens to a grade ≤3 CRS event within 24 hours of treating the symptoms of the grade 2 CRS event, the method may further involve administering to the subject an effective amount of an interleukin-6 receptor (IL-6R) antagonist (e.g., an anti-IL-6R antibody, e.g., tocilizumab (ACTEMRA®/ROACTEMRA®)) to manage the grade 2 or grade ≥3 CRS event. In some instances, tocilizumab is administered intravenously to the subject as a single dose of about 8 mg/kg. Other anti-IL-6R antibodies that could be used in combination with tocilizumab include sarilumab, vobarilizumab (ALX-0061), SA-237, and variants thereof.

If the subject has a grade 2 CRS event in the presence of extensive comorbidities following administration of the therapeutic bispecific antibody, the method may further include administering to the subject a first dose of an IL-6R antagonist (e.g., an anti-IL-6R antibody, e.g., tocilizumab (ACTEMRA®/ROACTEMRA®)) to manage the grade 2 CRS event while suspending treatment with the bispecific antibody. In some instances, the first dose of tocilizumab is administered intravenously to the subject at a dose of about 8 mg/kg. Other anti-IL-6R antibodies that could be used in combination with tocilizumab include sarilumab, vobarilizumab (ALX-0061), SA-237, and variants thereof. In some instances, if the grade 2 CRS event resolves to a grade ≤1 CRS event within two weeks, the method further includes resuming treatment with the bispecific antibody at a reduced dose. In some instances, the reduced dose is 50% of the initial infusion rate of the previous cycle if the event occurred during or within 24 hours of the infusion. If, on the other hand, the grade 2 CRS event does not resolve or worsens to a grade ≤3 CRS event within 24 hours of treating the symptoms of the grade 2 CRS event, the method may further include administering to the subject one or more (e.g., one, two, three, four, or five or more) additional doses of an IL-6R antagonist (e.g., an anti-IL-6R antibody, e.g., tocilizumab) to manage the grade 2 or grade ≤3 CRS event. In some particular instances, the grade 2 CRS event does not resolve or worsens to a grade ≤3 CRS event within 24 hours of treating the symptoms of the grade 2 CRS event, and the method may further include administering to the subject one or more additional doses of tocilizumab to manage the grade 2 or grade ≤3 CRS event. In some instances, the one or more additional doses of tocilizumab is administered intravenously to the subject at a dose of about 1 mg/kg to about 15 mg/kg, e.g., about 4 mg/kg to about 10 mg/kg, e.g., about 6 mg/kg to about 10 mg/kg, e.g., about 8 mg/kg. In some instances, the method further includes administering to the subject an effective amount of a corticosteroid. The corticosteroid may be administered before, after, or concurrently with the one or more additional doses of tocilizumab or other anti-IL-6R antibody. In some instances, the corticosteroid is administered intravenously to the subject. In some instances, the corticosteroid is methylprednisolone. In some instances, the methylprednisolone is administered at a dose of about 1 mg/kg per day to about 5 mg/kg per day, e.g., about 2 mg/kg per day. In some instances, the corticosteroid is dexamethasone. In some instances, the dexamethasone is administered at a dose of about 10 mg (e.g., a single dose of about 10 mg intravenously) or at a dose of about 0.5 mg/kg/day.

x. Management of Grade 3 CRS Events

If the subject has a grade 3 CRS event following administration of the therapeutic bispecific antibody, the method may further include administering to the subject a first dose of an IL-6R antagonist (e.g., an anti-IL-6R antibody, e.g., tocilizumab (ACTEMRA®/ROACTEMRA®)) to manage the grade 3 CRS event while suspending treatment with the bispecific antibody. In some instances, the first dose of tocilizumab is administered intravenously to the subject at a dose of about 8 mg/kg. Other anti-IL-6R antibodies that could be used in combination with tocilizumab include sarilumab, vobarilizumab (ALX-0061), SA-237, and variants thereof. In some instances, the subject recovers (e.g., is afebrile and off vasopressors) within 8 hours following treatment with the bispecific antibody, and the method further includes resuming treatment with the bispecific antibody at a reduced dose. In some instances, the reduced dose is 50% of the initial infusion rate of the previous cycle if the event occurred during or within 24 hours of the infusion. In other instances, if the grade 3 CRS event does not resolve or worsens to a grade 4 CRS event within 24 hours of treating the symptoms of the grade 3 CRS event, the method may further include administering to the subject one or more (e.g., one, two, three, four, or five or more) additional doses of an IL-6R antagonist (e.g., an anti-IL-6R antibody, e.g., tocilizumab) to manage the grade 3 or grade 4 CRS event. In some particular instances, the grade 3 CRS event does not resolve or worsens to a grade 4 CRS event within 24 hours of treating the symptoms of the grade 3 CRS event, and the method further includes administering to the subject one or more additional doses of tocilizumab to manage the grade 3 or grade 4 CRS event. In some instances, the one or more additional doses of tocilizumab is administered intravenously to the subject at a dose of about 1 mg/kg to about 15 mg/kg, e.g., about 4 mg/kg to about 10 mg/kg, e.g., about 6 mg/kg to about 10 mg/kg, e.g., about 8 mg/kg. In some instances, the method further includes administering to the subject an effective amount of a corticosteroid. The corticosteroid may be administered before, after, or concurrently with the one or more additional doses of tocilizumab or other anti-IL-6R antibody. In some instances, the corticosteroid is administered intravenously to the subject. In some instances, the corticosteroid is methylprednisolone. In some instances, the methylprednisolone is administered at a dose of about 1 mg/kg per day to about 5 mg/kg per day, e.g., about 2 mg/kg per day. In some instances, the corticosteroid is dexamethasone. In some instances, the dexamethasone is administered at a dose of about 10 mg (e.g., a single dose of about 10 mg intravenously) or at a dose of about 0.5 mg/kg/day.

xi. Management of Grade 4 CRS Events

If the subject has a grade 4 CRS event following administration of the therapeutic bispecific antibody, the method may further include administering to the subject a first dose of an IL-6R antagonist (e.g., an anti-IL-6R antibody, e.g., tocilizumab (ACTEMRA®/ROACTEMRA®)) to manage the grade 4 CRS event and permanently discontinuing treatment with the bispecific antibody. In some instances, the first dose of tocilizumab is administered intravenously to the subject at a dose of about 8 mg/kg. Other anti-IL-6R antibodies that could be used in combination with tocilizumab include sarilumab, vobarilizumab (ALX-0061), SA-237, and variants thereof. The grade 4 CRS event may, in some instances, resolve within 24 of treating the symptoms of the grade 4 CRS event. If the grade 4 CRS event does not resolve within 24 hours of treating the symptoms of the grade 4 CRS event, the method may further include administering to the subject one or more additional doses of an IL-6R antagonist (e.g., an anti-IL-6R antibody, e.g., tocilizumab (ACTEMRA®/ROACTEMRA®)) to manage the grade 4 CRS event. In some particular instances, the grade 4 CRS event does not resolve within 24 hours of treating the symptoms of the grade 4 CRS event, and the method further includes administering to the subject one or more (e.g., one, two, three, four, or five or more) additional doses of tocilizumab to manage the grade 4 CRS event. In some instances, the one or more additional doses of tocilizumab is administered intravenously to the subject at a dose of about 1 mg/kg to about 15 mg/kg, e.g., about 4 mg/kg to about 10 mg/kg, e.g., about 6 mg/kg to about 10 mg/kg, e.g., about 8 mg/kg. In some instances, the method further includes administering to the subject an effective amount of a corticosteroid. The corticosteroid may be administered before, after, or concurrently with the one or more additional doses of tocilizumab or another anti-IL-6R antibody. In some instances, the corticosteroid is administered intravenously to the subject. In some instances, the corticosteroid is methylprednisolone. In some instances, the methylprednisolone is administered at a dose of about 1 mg/kg per day to about 5 mg/kg per day, e.g., about 2 mg/kg per day. In some instances, the corticosteroid is dexamethasone. In some instances, the dexamethasone is administered at a dose of about 10 mg (e.g., a single dose of about 10 mg intravenously) or at a dose of about 0.5 mg/kg/day.

xii. Acetaminophen or Paracetamol

In another instance, the additional therapeutic agent is an effective amount of acetaminophen or paracetamol. The acetaminophen or paracetamol may be administered orally to the subject, e.g., administered orally at a dose of between about 500 mg to about 1000 mg. In some aspects, the acetaminophen or paracetamol is administered to the subject as a premedication, e.g., is administered prior to the administration of the bispecific anti-FcRH5/anti-CD3 antibody.

xiii. Diphenhydramine

In another instance, the additional therapeutic agent is an effective amount of diphenhydramine. The diphenhydramine may be administered orally to the subject, e.g., administered orally at a dose of between about 25 mg to about 50 mg. In some aspects, the diphenhydramine is administered to the subject as a premedication, e.g., is administered prior to the administration of the bispecific anti-FcRH5/anti-CD3 antibody.

xiv. Anti-Myeloma Agents

In another instance, the additional therapeutic agent is an effective amount of an anti-myeloma agent, e.g., an anti-myeloma agent that augments and/or complements T-cell-mediated killing of myeloma cells. The anti-myeloma agent may be, e.g., pomalidomide, daratumumab, and/or a B-cell maturation antigen (BCMA)-directed therapy (e.g., an antibody-drug conjugate targeting BCMA (BCMA-ADC)). In some aspects, the anti-myeloma agent is administered in four-week cycles.

xv. Premedication with Corticosteroids, Acetaminophen or Paracetamol, and/or Diphenhydramine

Any of the methods or treatments disclosed herein may include premedication with a corticosteroid prior to administration of the bispecific antibody to the subject.

Any of the methods or treatments disclosed herein may include premedication with acetaminophen or paracetamol prior to administration of the bispecific antibody to the subject.

Any of the methods or treatments disclosed herein may include premedication with diphenhydramine prior to administration of the bispecific antibody to the subject.

For example, any of the methods or treatments disclosed herein may include premedication with the following agents prior to administration of the bispecific antibody to the subject: (i) a corticosteroid; (ii) acetaminophen or paracetamol; and/or (iii) diphenhydramine.

In some examples, the method of treatment comprises a first phase, and the corticosteroid is administered to the subject 1 hour (±15 minutes) prior to any administration of the bispecific antibody during the first phase.

In some examples, the method of treatment comprises a first phase, and the corticosteroid is administered to the subject 24 hours prior to any administration of the bispecific antibody during the first phase.

In some examples, the method or treatment comprises a second phase, wherein the subject has experienced CRS with a prior administration of the bispecific, and the corticosteroid is administered to the subject 1 hour (±15 minutes) prior to any administration of the bispecific antibody during the second phase.

Any suitable corticosteroid may be used. In some examples, the corticosteroid is dexamethasone or methylprednisolone. In some examples, corticosteroid is dexamethasone. In some examples, the dexamethasone is administered to the subject at a dose of about 20 mg. In some examples, corticosteroid is methylprednisolone. In some examples, the methylprednisolone is administered to the subject at a dose of about 80 mg.

The corticosteroid may be administered by any suitable route. In some examples, the corticosteroid is administered to the subject intravenously.

In some examples, acetaminophen or paracetamol is administered to the subject at a dose of between 500 mg to 1000 mg. In some examples, acetaminophen or paracetamol is administered to the subject orally.

In some examples, diphenhydramine is administered to the subject at a dose of between 25 mg to 50 mg. In some examples, diphenhydramine is administered to the subject orally.

xvi. Other Combination Therapies

In some aspects, the one or more additional therapeutic agents comprise a PD-1 axis binding antagonist, an immunomodulatory agent, an anti-neoplastic agent, a chemotherapeutic agent, a growth inhibitory agent, an anti-angiogenic agent, a radiation therapy, a cytotoxic agent, a cell-based therapy, or a combination thereof.

xvii. PD-1 Axis Binding Antagonists

In some aspects, the additional therapeutic agent is a PD-1 axis binding antagonist. PD-1 axis binding antagonists may include PD-L1 binding antagonists, PD-1 binding antagonists, and PD-L2 binding antagonists. Any suitable PD-1 axis binding antagonist may be used.

In some instances, the PD-L1 binding antagonist inhibits the binding of PD-L1 to one or more of its ligand binding partners. In other instances, the PD-L1 binding antagonist inhibits the binding of PD-L1 to PD-1. In yet other instances, the PD-L1 binding antagonist inhibits the binding of PD-L1 to B7-1. In some instances, the PD-L1 binding antagonist inhibits the binding of PD-L1 to both PD-1 and B7-1. The PD-L1 binding antagonist may be, without limitation, an antibody, an antigen-binding fragment thereof, an immunoadhesin, a fusion protein, an oligopeptide, or a small molecule. In some instances, the PD-L1 binding antagonist is a small molecule that inhibits PD-L1 (e.g., GS-4224, INCB086550, MAX-10181, INCB090244, CA-170, or ABSK041). In some instances, the PD-L1 binding antagonist is a small molecule that inhibits PD-L1 and VISTA. In some instances, the PD-L1 binding antagonist is CA-170 (also known as AUPM-170). In some instances, the PD-L1 binding antagonist is a small molecule that inhibits PD-L1 and TIM3. In some instances, the small molecule is a compound described in WO 2015/033301 and/or WO 2015/033299.

In some instances, the PD-L1 binding antagonist is an anti-PD-L1 antibody. A variety of anti-PD-L1 antibodies are contemplated and described herein. In any of the instances herein, the isolated anti-PD-L1 antibody can bind to a human PD-L1, for example a human PD-L1 as shown in UniProtKB/Swiss-Prot Accession No. Q9NZQ7-1, or a variant thereof. In some instances, the anti-PD-L1 antibody is capable of inhibiting binding between PD-L1 and PD-1 and/or between PD-L1 and B7-1. In some instances, the anti-PD-L1 antibody is a monoclonal antibody. In some instances, the anti-PD-L1 antibody is an antibody fragment selected from the group consisting of Fab, Fab′-SH, Fv, scFv, and (Fab′)₂ fragments. In some instances, the anti-PD-L1 antibody is a humanized antibody. In some instances, the anti-PD-L1 antibody is a human antibody. Exemplary anti-PD-L1 antibodies include atezolizumab, MDX-1105, MEDI4736 (durvalumab), MSB0010718C (avelumab), SHR-1316, CS1001, envafolimab, TQB2450, ZKAB001, LP-002, CX-072, IMC-001, KL-A167, APL-502, cosibelimab, lodapolimab, FAZ053, TG-1501, BGB-A333, BCD-135, AK-106, LDP, GR1405, HLX20, MSB2311, RC98, PDL-GEX, KD036, KY1003, YBL-007, and HS-636. In some instances, the anti-PD-L1 antibody is atezolizumab. Examples of anti-PD-L1 antibodies useful in the methods of this invention and methods of making them are described in International Patent Application Publication No. WO 2010/077634 and U.S. Pat. No. 8,217,149, each of which is incorporated herein by reference in its entirety.

In some instances, the anti-PD-L1 antibody is avelumab (CAS Registry Number: 1537032-82-8). Avelumab, also known as MSB0010718C, is a human monoclonal IgG1 anti-PD-L1 antibody (Merck KGaA, Pfizer).

In some instances, the anti-PD-L1 antibody is durvalumab (CAS Registry Number: 1428935-60-7). Durvalumab, also known as MEDI4736, is an Fc-optimized human monoclonal IgG1 kappa anti-PD-L1 antibody (MedImmune, AstraZeneca) described in WO 2011/066389 and US 2013/034559.

In some instances, the anti-PD-L1 antibody is MDX-1105 (Bristol Myers Squibb). MDX-1105, also known as BMS-936559, is an anti-PD-L1 antibody described in WO 2007/005874.

In some instances, the anti-PD-L1 antibody is LY3300054 (Eli Lilly).

In some instances, the anti-PD-L1 antibody is STI-A1014 (Sorrento). STI-A1014 is a human anti-PD-L1 antibody.

In some instances, the anti-PD-L1 antibody is KN035 (Suzhou Alphamab). KN035 is single-domain antibody (dAB) generated from a camel phage display library.

In some instances, the anti-PD-L1 antibody comprises a cleavable moiety or linker that, when cleaved (e.g., by a protease in the tumor microenvironment), activates an antibody antigen binding domain to allow it to bind its antigen, e.g., by removing a non-binding steric moiety. In some instances, the anti-PD-L1 antibody is CX-072 (CytomX Therapeutics).

In some instances, the anti-PD-L1 antibody comprises the six HVR sequences (e.g., the three heavy chain HVRs and the three light chain HVRs) and/or the heavy chain variable domain and light chain variable domain from an anti-PD-L1 antibody described in US 20160108123, WO 2016/000619, WO 2012/145493, U.S. Pat. No. 9,205,148, WO 2013/181634, or WO 2016/061142.

In some instances, the PD-1 axis binding antagonist is a PD-1 binding antagonist. For example, in some instances, the PD-1 binding antagonist inhibits the binding of PD-1 to one or more of its ligand binding partners. In some instances, the PD-1 binding antagonist inhibits the binding of PD-1 to PD-L1. In other instances, the PD-1 binding antagonist inhibits the binding of PD-1 to PD-L2. In yet other instances, the PD-1 binding antagonist inhibits the binding of PD-1 to both PD-L1 and PD-L2. The PD-1 binding antagonist may be, without limitation, an antibody, an antigen-binding fragment thereof, an immunoadhesin, a fusion protein, an oligopeptide, or a small molecule. In some instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence). For example, in some instances, the PD-1 binding antagonist is an Fc-fusion protein. In some instances, the PD-1 binding antagonist is AMP-224. AMP-224, also known as B7-DCIg, is a PD-L2-Fc fusion soluble receptor described in WO 2010/027827 and WO 2011/066342. In some instances, the PD-1 binding antagonist is a peptide or small molecule compound. In some instances, the PD-1 binding antagonist is AUNP-12 (PierreFabre/Aurigene). See, e.g., WO 2012/168944, WO 2015/036927, WO 2015/044900, WO 2015/033303, WO 2013/144704, WO 2013/132317, and WO 2011/161699. In some instances, the PD-1 binding antagonist is a small molecule that inhibits PD-1.

In some instances, the PD-1 binding antagonist is an anti-PD-1 antibody. A variety of anti-PD-1 antibodies can be utilized in the methods and uses disclosed herein. In any of the instances herein, the PD-1 antibody can bind to a human PD-1 or a variant thereof. In some instances, the anti-PD-1 antibody is a monoclonal antibody. In some instances, the anti-PD-1 antibody is an antibody fragment selected from the group consisting of Fab, Fab′, Fab′-SH, Fv, scFv, and (Fab′)₂ fragments. In some instances, the anti-PD-1 antibody is a humanized antibody. In other instances, the anti-PD-1 antibody is a human antibody. Exemplary anti-PD-1 antagonist antibodies include nivolumab, pembrolizumab, MEDI-0680, PDR001 (spartalizumab), REGN2810 (cemiplimab), BGB-108, prolgolimab, camrelizumab, sintilimab, tislelizumab, toripalimab, dostarlimab, retifanlimab, sasanlimab, penpulimab, CS1003, HLX10, SCT-I10A, zimberelimab, balstilimab, genolimzumab, BI 754091, cetrelimab, YBL-006, BAT1306, HX008, budigalimab, AMG 404, CX-188, JTX-4014, 609A, Sym021, LZM009, F520, SG001, AM0001, ENUM 244C8, ENUM 388D4, STI-1110, AK-103, and hAb21.

In some instances, the anti-PD-1 antibody is nivolumab (CAS Registry Number: 946414-94-4). Nivolumab (Bristol-Myers Squibb/Ono), also known as MDX-1106-04, MDX-1106, ONO-4538, BMS-936558, and OPDIVO®, is an anti-PD-1 antibody described in WO 2006/121168.

In some instances, the anti-PD-1 antibody is pembrolizumab (CAS Registry Number: 1374853-91-4). Pembrolizumab (Merck), also known as MK-3475, Merck 3475, lambrolizumab, SCH-900475, and KEYTRUDA®, is an anti-PD-1 antibody described in WO 2009/114335.

In some instances, the anti-PD-1 antibody is MEDI-0680 (AMP-514; AstraZeneca). MEDI-0680 is a humanized IgG4 anti-PD-1 antibody.

In some instances, the anti-PD-1 antibody is PDR001 (CAS Registry No. 1859072-53-9; Novartis). PDR001 is a humanized IgG4 anti-PD-1 antibody that blocks the binding of PD-L1 and PD-L2 to PD-1.

In some instances, the anti-PD-1 antibody is REGN2810 (Regeneron). REGN2810 is a human anti-PD-1 antibody.

In some instances, the anti-PD-1 antibody is BGB-108 (BeiGene).

In some instances, the anti-PD-1 antibody is BGB-A317 (BeiGene).

In some instances, the anti-PD-1 antibody is JS-001 (Shanghai Junshi). JS-001 is a humanized anti-PD-1 antibody.

In some instances, the anti-PD-1 antibody is STI-A1110 (Sorrento). STI-A1110 is a human anti-PD-1 antibody.

In some instances, the anti-PD-1 antibody is INCSHR-1210 (Incyte). INCSHR-1210 is a human IgG4 anti-PD-1 antibody.

In some instances, the anti-PD-1 antibody is PF-06801591 (Pfizer).

In some instances, the anti-PD-1 antibody is TSR-042 (also known as ANB011; Tesaro/AnaptysBio).

In some instances, the anti-PD-1 antibody is AM0001 (ARMO Biosciences).

In some instances, the anti-PD-1 antibody is ENUM 244C8 (Enumeral Biomedical Holdings). ENUM 244C8 is an anti-PD-1 antibody that inhibits PD-1 function without blocking binding of PD-L1 to PD-1.

In some instances, the anti-PD-1 antibody is ENUM 388D4 (Enumeral Biomedical Holdings). ENUM 388D4 is an anti-PD-1 antibody that competitively inhibits binding of PD-L1 to PD-1.

In some instances, the anti-PD-1 antibody comprises the six HVR sequences (e.g., the three heavy chain HVRs and the three light chain HVRs) and/or the heavy chain variable domain and light chain variable domain from an anti-PD-1 antibody described in WO 2015/112800, WO 2015/112805, WO 2015/112900, US 20150210769, WO2016/089873, WO 2015/035606, WO 2015/085847, WO 2014/206107, WO 2012/145493, U.S. Pat. No. 9,205,148, WO 2015/119930, WO 2015/119923, WO 2016/032927, WO 2014/179664, WO 2016/106160, and WO 2014/194302.

In some instances, the PD-1 axis binding antagonist is a PD-L2 binding antagonist. In some instances, the PD-L2 binding antagonist is a molecule that inhibits the binding of PD-L2 to its ligand binding partners. In a specific aspect, the PD-L2 binding ligand partner is PD-1. The PD-L2 binding antagonist may be, without limitation, an antibody, an antigen-binding fragment thereof, an immunoadhesin, a fusion protein, an oligopeptide, or a small molecule.

In some instances, the PD-L2 binding antagonist is an anti-PD-L2 antibody. In any of the instances herein, the anti-PD-L2 antibody can bind to a human PD-L2 or a variant thereof. In some instances, the anti-PD-L2 antibody is a monoclonal antibody. In some instances, the anti-PD-L2 antibody is an antibody fragment selected from the group consisting of Fab, Fab′, Fab′-SH, Fv, scFv, and (Fab′)₂ fragments. In some instances, the anti-PD-L2 antibody is a humanized antibody. In other instances, the anti-PD-L2 antibody is a human antibody. In a still further specific aspect, the anti-PD-L2 antibody has reduced or minimal effector function. In a still further specific aspect, the minimal effector function results from an “effector-less Fc mutation” or aglycosylation mutation. In still a further instance, the effector-less Fc mutation is an N297A or D265A/N297A substitution in the constant region. In some instances, the isolated anti-PD-L2 antibody is aglycosylated.

xviii. Growth Inhibitory Agents

In some aspects, the additional therapeutic agent is a growth inhibitory agent. Exemplary growth inhibitory agents include agents that block cell cycle progression at a place other than S phase, e.g., agents that induce G1 arrest (e.g., DNA alkylating agents such as tamoxifen, prednisone, dacarbazine, mechlorethamine, cisplatin, methotrexate, 5-fluorouracil, or ara-C) or M-phase arrest (e.g., vincristine, vinblastine, taxanes (e.g., paclitaxel and docetaxel), doxorubicin, epirubicin, daunorubicin, etoposide, or bleomycin).

xix. Radiation Therapies

In some aspects, the additional therapeutic agent is a radiation therapy. Radiation therapies include the use of directed gamma rays or beta rays to induce sufficient damage to a cell so as to limit its ability to function normally or to destroy the cell altogether. Typical treatments are given as a one-time administration and typical dosages range from 10 to 200 units (Grays) per day.

xx. Cytotoxic Agents

In some aspects, the additional therapeutic agent is a cytotoxic agent, e.g., a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹², and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and antitumor or anticancer agents.

xxi. Anti-Cancer Therapies

In some instances, the methods include administering to the individual an anti-cancer therapy other than, or in addition to, a bispecific anti-FcRH5/anti-CD3 antibody (e.g., an anti-neoplastic agent, a chemotherapeutic agent, a growth inhibitory agent, an anti-angiogenic agent, a radiation therapy, or a cytotoxic agent).

In some instances, the methods further involve administering to the patient an effective amount of an additional therapeutic agent. In some instances, the additional therapeutic agent is selected from the group consisting of an anti-neoplastic agent, a chemotherapeutic agent, a growth inhibitory agent, an anti-angiogenic agent, a radiation therapy, a cytotoxic agent, and combinations thereof. In some instances, a bispecific anti-FcRH5/anti-CD3 antibody may be administered in conjunction with a chemotherapy or chemotherapeutic agent. In some instances, a bispecific anti-FcRH5/anti-CD3 antibody may be administered in conjunction with a radiation therapy agent. In some instances, a bispecific anti-FcRH5/anti-CD3 antibody may be administered in conjunction with a targeted therapy or targeted therapeutic agent. In some instances, a bispecific anti-FcRH5/anti-CD3 antibody may be administered in conjunction with an immunotherapy or immunotherapeutic agent, for example a monoclonal antibody. In some instances, the additional therapeutic agent is an agonist directed against a co-stimulatory molecule. In some instances, the additional therapeutic agent is an antagonist directed against a co-inhibitory molecule.

Without wishing to be bound to theory, it is thought that enhancing T-cell stimulation, by promoting a co-stimulatory molecule or by inhibiting a co-inhibitory molecule, may promote tumor cell death thereby treating or delaying progression of cancer. In some instances, a bispecific anti-FcRH5/anti-CD3 antibody may be administered in conjunction with an agonist directed against a co-stimulatory molecule. In some instances, a co-stimulatory molecule may include CD40, CD226, CD28, OX40, GITR, CD137, CD27, HVEM, or CD127. In some instances, the agonist directed against a co-stimulatory molecule is an agonist antibody that binds to CD40, CD226, CD28, OX40, GITR, CD137, CD27, HVEM, or CD127. In some instances, a bispecific anti-FcRH5/anti-CD3 antibody may be administered in conjunction with an antagonist directed against a co-inhibitory molecule. In some instances, a co-inhibitory molecule may include CTLA-4 (also known as CD152), TIM-3, BTLA, VISTA, LAG-3, B7-H3, B7-H4, IDO, TIGIT, MICA/B, or arginase. In some instances, the antagonist directed against a co-inhibitory molecule is an antagonist antibody that binds to CTLA-4, TIM-3, BTLA, VISTA, LAG-3, B7-H3, B7-H4, IDO, TIGIT, MICA/B, or arginase.

In some instances, a bispecific anti-FcRH5/anti-CD3 antibody may be administered in conjunction with an antagonist directed against CTLA-4 (also known as CD152), e.g., a blocking antibody. In some instances, a bispecific anti-FcRH5/anti-CD3 antibody may be administered in conjunction with ipilimumab (also known as MDX-010, MDX-101, or YERVOY®). In some instances, a bispecific anti-FcRH5/anti-CD3 antibody may be administered in conjunction with tremelimumab (also known as ticilimumab or CP-675,206). In some instances, a bispecific anti-FcRH5/anti-CD3 antibody may be administered in conjunction with an antagonist directed against B7-H3 (also known as CD276), e.g., a blocking antibody. In some instances, a bispecific anti-FcRH5/anti-CD3 antibody may be administered in conjunction with MGA271. In some instances, a bispecific anti-FcRH5/anti-CD3 antibody may be administered in conjunction with an antagonist directed against a TGF-beta, e.g., metelimumab (also known as CAT-192), fresolimumab (also known as GC1008), or LY2157299.

In some instances, a bispecific anti-FcRH5/anti-CD3 antibody may be administered in conjunction with a treatment comprising adoptive transfer of a T-cell (e.g., a cytotoxic T-cell or CTL) expressing a chimeric antigen receptor (CAR). In some instances, bispecific anti-FcRH5/anti-CD3 antibody may be administered in conjunction with a treatment comprising adoptive transfer of a T-cell comprising a dominant-negative TGF beta receptor, e.g., a dominant-negative TGF beta type II receptor. In some instances, a bispecific anti-FcRH5/anti-CD3 antibody may be administered in conjunction with a treatment comprising a HERCREEM protocol (see, e.g., ClinicalTrials.gov Identifier NCT00889954).

In some instances, a bispecific anti-FcRH5/anti-CD3 antibody may be administered in conjunction with an agonist directed against CD137 (also known as TNFRSF9, 4-1BB, or ILA), e.g., an activating antibody. In some instances, a bispecific anti-FcRH5/anti-CD3 antibody may be administered in conjunction with urelumab (also known as BMS-663513). In some instances, a bispecific anti-FcRH5/anti-CD3 antibody may be administered in conjunction with an agonist directed against CD40, e.g., an activating antibody. In some instances, bispecific anti-FcRH5/anti-CD3 antibody may be administered in conjunction with CP-870893. In some instances, bispecific anti-FcRH5/anti-CD3 antibody may be administered in conjunction with an agonist directed against OX40 (also known as CD134), e.g., an activating antibody. In some instances, a bispecific anti-FcRH5/anti-CD3 antibody may be administered in conjunction with an anti-OX40 antibody (e.g., AgonOX). In some instances, a bispecific anti-FcRH5/anti-CD3 antibody may be administered in conjunction with an agonist directed against CD27, e.g., an activating antibody. In some instances, a bispecific anti-FcRH5/anti-CD3 antibody may be administered in conjunction with CDX-1127. In some instances, a bispecific anti-FcRH5/anti-CD3 antibody may be administered in conjunction with an antagonist directed against indoleamine-2,3-dioxygenase (IDO). In some instances, the IDO antagonist is 1-methyl-D-tryptophan (also known as 1-D-MT).

In some instances, a bispecific anti-FcRH5/anti-CD3 antibody may be administered in conjunction with an antibody-drug conjugate. In some instances, the antibody-drug conjugate comprises mertansine or monomethyl auristatin E (MMAE). In some instances, a bispecific anti-FcRH5/anti-CD3 antibody may be administered in conjunction with an anti-NaPi2b antibody-MMAE conjugate (also known as DNIB0600A or RG7599). In some instances, a bispecific anti-FcRH5/anti-CD3 antibody may be administered in conjunction with trastuzumab emtansine (also known as T-DM1, ado-trastuzumab emtansine, or KADCYLA®, Genentech). In some instances, a bispecific anti-FcRH5/anti-CD3 antibody may be administered in conjunction with DMUC5754A. In some instances, a bispecific anti-FcRH5/anti-CD3 antibody may be administered in conjunction with an antibody-drug conjugate targeting the endothelin B receptor (EDNBR), e.g., an antibody directed against EDNBR conjugated with MMAE.

In some instances, a bispecific anti-FcRH5/anti-CD3 antibody may be administered in conjunction with an anti-angiogenesis agent. In some instances, a bispecific anti-FcRH5/anti-CD3 antibody may be administered in conjunction with an antibody directed against a VEGF, e.g., VEGF-A. In some instances, a bispecific anti-FcRH5/anti-CD3 antibody may be administered in conjunction with bevacizumab (also known as AVASTIN®, Genentech). In some instances, a bispecific anti-FcRH5/anti-CD3 antibody may be administered in conjunction with an antibody directed against angiopoietin 2 (also known as Ang2). In some instances, a bispecific anti-FcRH5/anti-CD3 antibody may be administered in conjunction with MEDI3617.

In some instances, a bispecific anti-FcRH5/anti-CD3 antibody may be administered in conjunction with an antineoplastic agent. In some instances, a bispecific anti-FcRH5/anti-CD3 antibody may be administered in conjunction with an agent targeting CSF-1R (also known as M-CSFR or CD115). In some instances, a bispecific anti-FcRH5/anti-CD3 antibody may be administered in conjunction with anti-CSF-1R (also known as IMC-CS4). In some instances, a bispecific anti-FcRH5/anti-CD3 antibody may be administered in conjunction with an interferon, for example interferon alpha or interferon gamma. In some instances, a bispecific anti-FcRH5/anti-CD3 antibody may be administered in conjunction with Roferon-A (also known as recombinant Interferon alpha-2a). In some instances, a bispecific anti-FcRH5/anti-CD3 antibody may be administered in conjunction with GM-CSF (also known as recombinant human granulocyte macrophage colony stimulating factor, rhu GM-CSF, sargramostim, or LEUKINE®). In some instances, a bispecific anti-FcRH5/anti-CD3 antibody may be administered in conjunction with IL-2 (also known as aldesleukin or PROLEUKIN®). In some instances, a bispecific anti-FcRH5/anti-CD3 antibody may be administered in conjunction with IL-12. In some instances, a bispecific anti-FcRH5/anti-CD3 antibody may be administered in conjunction with an antibody targeting CD20. In some instances, the antibody targeting CD20 is obinutuzumab (also known as GA101 or GAZYVA®) or rituximab. In some instances, a bispecific anti-FcRH5/anti-CD3 antibody may be administered in conjunction with an antibody targeting GITR. In some instances, the antibody targeting GITR is TRX518.

In some instances, a bispecific anti-FcRH5/anti-CD3 antibody may be administered in conjunction with a cancer vaccine. In some instances, the cancer vaccine is a peptide cancer vaccine, which in some instances is a personalized peptide vaccine. In some instances, the peptide cancer vaccine is a multivalent long peptide, a multi-peptide, a peptide cocktail, a hybrid peptide, or a peptide-pulsed dendritic cell vaccine (see, e.g., Yamada et al., Cancer Sci. 104:14-21, 2013). In some instances, a bispecific anti-FcRH5/anti-CD3 antibody may be administered in conjunction with an adjuvant. In some instances, a bispecific anti-FcRH5/anti-CD3 antibody may be administered in conjunction with a treatment comprising a TLR agonist, e.g., Poly-ICLC (also known as HILTONOL®), LPS, MPL, or CpG ODN. In some instances, a bispecific anti-FcRH5/anti-CD3 antibody may be administered in conjunction with tumor necrosis factor (TNF) alpha. In some instances, a bispecific anti-FcRH5/anti-CD3 antibody may be administered in conjunction with IL-1. In some instances, a bispecific anti-FcRH5/anti-CD3 antibody may be administered in conjunction with HMGB1. In some instances, a bispecific anti-FcRH5/anti-CD3 antibody may be administered in conjunction with an IL-10 antagonist. In some instances, a bispecific anti-FcRH5/anti-CD3 antibody may be administered in conjunction with an IL-4 antagonist. In some instances, a bispecific anti-FcRH5/anti-CD3 antibody may be administered in conjunction with an IL-13 antagonist. In some instances, a bispecific anti-FcRH5/anti-CD3 antibody may be administered in conjunction with an HVEM antagonist. In some instances, a bispecific anti-FcRH5/anti-CD3 antibody may be administered in conjunction with an ICOS agonist, e.g., by administration of ICOS-L, or an agonistic antibody directed against ICOS. In some instances, a bispecific anti-FcRH5/anti-CD3 antibody may be administered in conjunction with a treatment targeting CX3CL1. In some instances, a bispecific anti-FcRH5/anti-CD3 antibody may be administered in conjunction with a treatment targeting CXCL9. In some instances, a bispecific anti-FcRH5/anti-CD3 antibody may be administered in conjunction with a treatment targeting CXCL10. In some instances, a bispecific anti-FcRH5/anti-CD3 antibody may be administered in conjunction with a treatment targeting CCL5. In some instances, a bispecific anti-FcRH5/anti-CD3 antibody may be administered in conjunction with an LFA-1 or ICAM1 agonist. In some instances, a bispecific anti-FcRH5/anti-CD3 antibody may be administered in conjunction with a Selectin agonist.

In some instances, a bispecific anti-FcRH5/anti-CD3 antibody may be administered in conjunction with a targeted therapy. In some instances, a bispecific anti-FcRH5/anti-CD3 antibody may be administered in conjunction with an inhibitor of B-Raf. In some instances, a bispecific anti-FcRH5/anti-CD3 antibody may be administered in conjunction with vemurafenib (also known as ZELBORAF®). In some instances, a bispecific anti-FcRH5/anti-CD3 antibody may be administered in conjunction with dabrafenib (also known as TAFINLAR®). In some instances, a bispecific anti-FcRH5/anti-CD3 antibody may be administered in conjunction with erlotinib (also known as TARCEVA®). In some instances, a bispecific anti-FcRH5/anti-CD3 antibody may be administered in conjunction with an inhibitor of a MEK, such as MEK1 (also known as MAP2K1) or MEK2 (also known as MAP2K2). In some instances, a bispecific anti-FcRH5/anti-CD3 antibody may be administered in conjunction with cobimetinib (also known as GDC-0973 or XL-518). In some instances, a bispecific anti-FcRH5/anti-CD3 antibody may be administered in conjunction with trametinib (also known as MEKINIST®). In some instances, a bispecific anti-FcRH5/anti-CD3 antibody may be administered in conjunction with an inhibitor of K-Ras. In some instances, a bispecific anti-FcRH5/anti-CD3 antibody may be administered in conjunction with an inhibitor of c-Met. In some instances, a bispecific anti-FcRH5/anti-CD3 antibody may be administered in conjunction with onartuzumab (also known as MetMAb). In some instances, a bispecific anti-FcRH5/anti-CD3 antibody may be administered in conjunction with an inhibitor of Alk. In some instances, a bispecific anti-FcRH5/anti-CD3 antibody may be administered in conjunction with AF802 (also known as CH5424802 or alectinib). In some instances, a bispecific anti-FcRH5/anti-CD3 antibody may be administered in conjunction with an inhibitor of a phosphatidylinositol 3-kinase (PI3K). In some instances, a bispecific anti-FcRH5/anti-CD3 antibody may be administered in conjunction with BKM120. In some instances, a bispecific anti-FcRH5/anti-CD3 antibody may be administered in conjunction with idelalisib (also known as GS-1101 or CAL-101). In some instances, a bispecific anti-FcRH5/anti-CD3 antibody may be administered in conjunction with perifosine (also known as KRX-0401). In some instances, a bispecific anti-FcRH5/anti-CD3 antibody may be administered in conjunction with an inhibitor of an Akt. In some instances, a bispecific anti-FcRH5/anti-CD3 antibody may be administered in conjunction with MK2206. In some instances, a bispecific anti-FcRH5/anti-CD3 antibody may be administered in conjunction with GSK690693. In some instances, a bispecific anti-FcRH5/anti-CD3 antibody may be administered in conjunction with GDC-0941. In some instances, a bispecific anti-FcRH5/anti-CD3 antibody may be administered in conjunction with an inhibitor of mTOR. In some instances, a bispecific anti-FcRH5/anti-CD3 antibody may be administered in conjunction with sirolimus (also known as rapamycin). In some instances, a bispecific anti-FcRH5/anti-CD3 antibody may be administered in conjunction with temsirolimus (also known as CCI-779 or TORISEL®). In some instances, a bispecific anti-FcRH5/anti-CD3 antibody may be administered in conjunction with everolimus (also known as RAD001). In some instances, a bispecific anti-FcRH5/anti-CD3 antibody may be administered in conjunction with ridaforolimus (also known as AP-23573, MK-8669, or deforolimus). In some instances, a bispecific anti-FcRH5/anti-CD3 antibody may be administered in conjunction with OSI-027. In some instances, a bispecific anti-FcRH5/anti-CD3 antibody may be administered in conjunction with AZD8055. In some instances, a bispecific anti-FcRH5/anti-CD3 antibody may be administered in conjunction with INK128. In some instances, a bispecific anti-FcRH5/anti-CD3 antibody may be administered in conjunction with a dual PI3K/mTOR inhibitor. In some instances, a bispecific anti-FcRH5/anti-CD3 antibody may be administered in conjunction with XL765. In some instances, a bispecific anti-FcRH5/anti-CD3 antibody may be administered in conjunction with GDC-0980. In some instances, a bispecific anti-FcRH5/anti-CD3 antibody may be administered in conjunction with BEZ235 (also known as NVP-BEZ235). In some instances, a bispecific anti-FcRH5/anti-CD3 antibody may be administered in conjunction with BGT226. In some instances, a bispecific anti-FcRH5/anti-CD3 antibody may be administered in conjunction with GSK2126458. In some instances, a bispecific anti-FcRH5/anti-CD3 antibody may be administered in conjunction with PF-04691502. In some instances, a bispecific anti-FcRH5/anti-CD3 antibody may be administered in conjunction with PF-05212384 (also known as PKI-587).

In some instances, a bispecific anti-FcRH5/anti-CD3 antibody may be administered in conjunction with a chemotherapeutic agent. A chemotherapeutic agent is a chemical compound useful in the treatment of cancer. Exemplary chemotherapeutic agents include, but are not limited to erlotinib (TARCEVA®, Genentech/OSI Pharm.), anti-hormonal agents that act to regulate or inhibit hormone action on tumors such as anti-estrogens and selective estrogen receptor modulators (SERMs), antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), or trastuzumab (HERCEPTIN®, Genentech), EGFR inhibitors (EGFR antagonists), tyrosine kinase inhibitors, and chemotherapeutic agents also include non-steroidal anti-inflammatory drugs (NSAIDs) with analgesic, antipyretic and anti-inflammatory effects.

In instances for which the methods described herein involve a combination therapy, such as a particular combination therapy noted above, the combination therapy encompasses the co-administration of the bispecific anti-FcRH5/anti-CD3 antibody with one or more additional therapeutic agents, and such co-administration may be combined administration (where two or more therapeutic agents are included in the same or separate formulations) or separate administration, in which case, administration of the bispecific anti-FcRH5/anti-CD3 antibody can occur prior to, simultaneously, and/or following, administration of the additional therapeutic agent or agents. In one embodiment, administration of the bispecific anti-FcRH5/anti-CD3 antibody and administration of an additional therapeutic agent or exposure to radiotherapy can occur within about one month, or within about one, two or three weeks, or within about one, two, three, four, five, or six days, of each other.

In some aspects, the subject does not have an increased risk of CRS (e.g., has not experienced Grade 3+ CRS during treatment with a bispecific antibody or CAR-T therapy; does not have detectable circulating plasma cells; and/or does not have extensive extramedullary disease).

### D. Cancers

Any of the methods of the invention described herein may be useful for treating cancer, such as a B cell proliferative disorder, including multiple myeloma (MM), which may be relapsed or refractory (R/R) MM. In some aspects, the patient has received at least three prior lines of treatment for the B cell proliferative disorder (e.g., MM), e.g., has received three, four, five, six, or more than six prior lines of treatment. In some aspects, the patient has received at least three prior lines of treatment for the B cell proliferative disorder, wherein the treatment is a 4L+ treatment. For example, the patient may have been exposed to a proteasome inhibitor (PI), an immunomodulatory drug (IMiD), an autologous stem cell transplant (ASCT), an anti-CD38 therapy (e.g., anti-CD38 antibody therapy, e.g., daratumumab therapy), a CAR-T therapy, or a therapy comprising a bispecific antibody. In some instances, the patient has been exposed to all three of PI, IMiD, and anti-CD38 therapy (in other words, triple-class refractory). Other examples of B cell proliferative disorders/malignancies amenable to treatment with a bispecific anti-FcRH5/anti-CD3 antibody in accordance with the methods described herein include, without limitation, non-Hodgkin's lymphoma (NHL), including diffuse large B cell lymphoma (DLBCL), which may be relapsed or refractory DLBCL, as well as other cancers including germinal-center B cell-like (GCB) diffuse large B cell lymphoma (DLBCL), activated B cell-like (ABC) DLBCL, follicular lymphoma (FL), mantle cell lymphoma (MCL), acute myeloid leukemia (AML), chronic lymphoid leukemia (CLL), marginal zone lymphoma (MZL), small lymphocytic leukemia (SLL), lymphoplasmacytic lymphoma (LL), Waldenstrom macroglobulinemia (WM), central nervous system lymphoma (CNSL), Burkitt's lymphoma (BL), B cell prolymphocytic leukemia, splenic marginal zone lymphoma, hairy cell leukemia, splenic lymphoma/leukemia, unclassifiable, splenic diffuse red pulp small B cell lymphoma, hairy cell leukemia variant, Waldenstrom macroglobulinemia, heavy chain diseases, a heavy chain disease, γ heavy chain disease, p heavy chain disease, plasma cell myeloma, solitary plasmacytoma of bone, extraosseous plasmacytoma, extranodal marginal zone lymphoma of mucosa-associated lymphoid tissue (MALT lymphoma), nodal marginal zone lymphoma, pediatric nodal marginal zone lymphoma, pediatric follicular lymphoma, primary cutaneous follicle centre lymphoma, T cell/histiocyte rich large B cell lymphoma, primary DLBCL of the CNS, primary cutaneous DLBCL, leg type, EBV-positive DLBCL of the elderly, DLBCL associated with chronic inflammation, lymphomatoid granulomatosis, primary mediastinal (thymic) large B cell lymphoma, intravascular large B cell lymphoma, ALK-positive large B cell lymphoma, plasmablastic lymphoma, large B cell lymphoma arising in HHV8-associated multicentric Castleman disease, primary effusion lymphoma: B cell lymphoma, unclassifiable, with features intermediate between DLBCL and Burkitt lymphoma, and B cell lymphoma, unclassifiable, with features intermediate between DLBCL and classical Hodgkin's lymphoma. Further examples of B cell proliferative disorders include, but are not limited to, multiple myeloma (MM); low grade/follicular NHL; small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; AIDS-related lymphoma; and acute lymphoblastic leukemia (ALL); chronic myeloblastic leukemia; and post-transplant lymphoproliferative disorder (PTLD). Further examples of cancer include, but are not limited to, carcinoma, lymphoma, blastoma, sarcoma, and leukemia or lymphoid malignancies, including B cell lymphomas. More particular examples of such cancers include, but are not limited to, low grade/follicular NHL; small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; AIDS-related lymphoma; and acute lymphoblastic leukemia (ALL); chronic myeloblastic leukemia; and post-transplant lymphoproliferative disorder (PTLD). Solid tumors that may by amenable to treatment with a bispecific anti-FcRH5/anti-CD3 antibody in accordance with the methods described herein include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer and gastrointestinal stromal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, superficial spreading melanoma, lentigo maligna melanoma, acral lentiginous melanomas, nodular melanomas, as well as abnormal vascular proliferation associated with phakomatoses, edema (such as that associated with brain tumors), Meigs's syndrome, brain, as well as head and neck cancer, and associated metastases. In certain embodiments, cancers that are amenable to treatment by the antibodies of the invention include breast cancer, colorectal cancer, rectal cancer, non-small cell lung cancer, glioblastoma, non-Hodgkins lymphoma (NHL), renal cell cancer, prostate cancer, liver cancer, pancreatic cancer, soft-tissue sarcoma, Kaposi's sarcoma, carcinoid carcinoma, head and neck cancer, ovarian cancer, and mesothelioma.

### E. Prior Anti-Cancer Therapy

In some aspects, the subject has previously been treated for the B cell proliferative disorder (e.g., MM). In some aspects, the subject has received at least one, two, three, four, five, six, seven, eight, nine, ten, eleven, twelve, thirteen, fourteen, fifteen, or more than fifteen lines of treatment for the B cell proliferative disorder. In some aspects, the patient has received at least one prior line of treatment for the B cell proliferative disorder, e.g., the treatment is a 2L+, 3L+, 4L+, 5L+, 6L+, 7L+, 8L+, 9L+, 10L+, 11L+, 12L+, 13L+, 14L+, or 15L+ treatment. In some aspects, the subject has received at least three prior lines of treatment for the B cell proliferative disorder (e.g., MM), e.g., the patient has received a 4L+ treatment, e.g., has received three, four, five, six, seven, eight, nine, ten, eleven, twelve, thirteen, fourteen, fifteen, or more than fifteen lines of treatment. In some aspects, the subject has relapsed or refractory (R/R) multiple myeloma (MM), e.g., a patient having an R/R MM who is receiving a 4L+ treatment for R/R MM. In some aspects, the patient is triple-class refractory.

In some aspects, the prior lines of treatment include one or more of a proteasome inhibitor (PI), e.g., bortezomib, carfilzomib, or ixazomib; an immunomodulatory drug (IMiD), e.g., thalidomide, lenalidomide, or pomalidomide; an autologous stem cell transplant (ASCT); an anti-CD38 agent, e.g., daratumumab (DARZALEX®) (U.S. Pat. No. 7,829,673 and U.S. Pub. No: 20160067205 A1), “MOR202” (U.S. Pat. No. 8,263,746), isatuximab (SAR-650984); a CAR-T therapy; a therapy comprising a bispecific antibody; an anti-SLAMF7 therapeutic agent (e.g., an anti-SLAMF7 antibody, e.g., elotuzumab); a nuclear export inhibitor (e.g., selinexor); and a histone deacetylase (HDAC) inhibitor (e.g., panobinostat). In some aspects, the prior lines of treatment include an antibody-drug conjugate (ADC). In some aspects, the prior lines of treatment include a B-cell maturation antigen (BCMA)-directed therapy, e.g., an antibody-drug conjugate targeting BCMA (BCMA-ADC). Exemplary BCMA-targeting TDB antibodies include teclistimab (JNJ-64007957), AM701, AMG 420 (BCMAxCD3 bispecific T-cell engager, BiTE®, Amgen), CC-93269 (BCMAxCD3 bispecific antibody, Celgene), elranatamab (BCMAxCD3 bispecific antibody, Pfizer Inc.), TNB-383B (TeneoBio/AbbVie), linvoseltamab (REGN5458-BCMAxCD3 bispecific antibody, Regeneron), alnuctamab (CC-93269-BMS), AFM26 (BCMAxCD16 tetravalent bispecific antibody, Affimed GmbH), and HPN217 (BCMAxALBxCD3 trispecific, Harpoon Therapeutics).

In some aspects, the prior lines of treatment include all three of a proteasome inhibitor (PI), an IMiD, and an anti-CD38 agent (e.g., daratumumab). In some aspects, the patient is triple-class refractory.

In some aspects, the B cell proliferative disorder (e.g., MM) is refractory to the lines of treatment, e.g., is refractory to one or more of daratumumab, a PI, an IMiD, an ASCT, an anti-CD38 agent, a CAR-T therapy, a therapy comprising a bispecific antibody, an anti-SLAMF7 therapeutic agent, a nuclear export inhibitor, a HDAC inhibitor, an ADC, or a BCMA-directed therapy. In some aspects, the B cell proliferative disorder (e.g., MM) is refractory to daratumumab.

### F. Risk-Benefit Profile

The methods described herein may result in an improved benefit-risk profile for patients having cancer (e.g., a multiple myeloma (MM), e.g., a relapsed or refractory (R/R) MM), e.g., a patient having an R/R MM who is receiving a 4L+ treatment for R/R MM, being treated with a bispecific anti-FcRH5/anti-CD3 antibody. In some instances, treatment using the methods described herein that result in administering the bispecific anti-FcRH5/anti-CD3 antibody in the context of a fractionated, dose-escalation dosing regimen may result in a reduction (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater) or complete inhibition (100% reduction) of undesirable events, such as cytokine-driven toxicities (e.g., cytokine release syndrome (CRS)), infusion-related reactions (IRRs), macrophage activation syndrome (MAS), neurologic toxicities, severe tumor lysis syndrome (TLS), neutropenia, thrombocytopenia, elevated liver enzymes, and/or central nervous system (CNS) toxicities, following treatment with a bispecific anti-FcRH5/anti-CD3 antibody using the fractionated, dose-escalation dosing regimen of the invention relative to treatment with a bispecific anti-FcRH5/anti-CD3 antibody using an non-fractioned dosing regimen.

### G. Safety and Efficacy

i. Safety

In some aspects, less than 15% (e.g., less than 14%, less than 13%, less than 12%, less than 11%, less than 10%, less than 9%, less than 8%, less than 7%, less than 6%, less than 5%, less than 4%, less than 3%, less than 2%, or less than 1%) of patients treated using the methods described herein experience Grade 3 or Grade 4 cytokine release syndrome (CRS). In some aspects, less than 5% of patients treated using the methods described herein experience Grade 3 or Grade 4 CRS.

In some aspects, less than 10% (e.g., less than 9%, less than 8%, less than 7%, less than 6%, less than 5%, less than 4%, less than 3%, less than 2%, or less than 1%) of patients treated using the methods described herein experience Grade 4+ CRS. In some aspects, less than 3% of patients treated using the methods described herein experience Grade 4+ CRS. In some aspects, no patients experience Grade 4+ CRS.

In some aspects, less than 10% (e.g., less than 9%, less than 8%, less than 7%, less than 6%, less than 5%, less than 4%, less than 3%, less than 2%, or less than 1%) of patients treated using the methods described herein experience Grade 3 CRS. In some aspects, less than 5% of patients treated using the methods described herein experience Grade 3 CRS. In some aspects, no patients experience Grade 3 CRS.

In some aspects, Grade 2+ CRS events occur only in the first cycle of treatment. In some aspects, Grade 2 CRS events occur only in the first cycle of treatment. In some aspects, Grade 2 CRS events do not occur.

In some aspects, less than 3% of patients treated using the methods described herein experience Grade 4+ CRS, less than 5% of patients treated using the methods described herein experience Grade 3 CRS, and Grade 2+ CRS events occur only in the first cycle of treatment.

In some aspects, no Grade 3+ CRS events occur and Grade 2 CRS events occur only in the first cycle of treatment.

In some aspects, symptoms of immune effector cell-associated neurotoxicity syndrome (ICANS) are limited to confusion, disorientation, and expressive aphasia and resolve with steroids.

In some aspects, less than 10% (e.g., less than 9%, less than 8%, less than 7%, less than 6%, less than 5%, less than 4%, less than 3%, less than 2%, or less than 1%) of patients treated using the methods described herein experience seizures or other Grade 3+ neurologic adverse events. In some aspects, less than 5% of patients experience seizures or other Grade 3+ neurologic adverse events. In some aspects, no patients experience seizures or other Grade 3+ neurologic adverse events.

In some aspects, all neurological symptoms are either self-limited or resolved with steroids and/or tocilizumab therapy.

ii. Efficacy

In some aspects, the overall response rate (ORR) for patients treated using the methods described herein is at least 25%, e.g., is at least 30%, 35%, 40%, 45%, 50%, 55%, 60%, 65%, 70%, 75%, 80%, 85%, 90%, 95%, 99%, or 100%. In some aspects, the ORR is at least 40%. In some aspects, the ORR is at least 45% (e.g., at least 45%, 45.5%, 46%, 46.5% 47%, 47.5%, 48%, 48.5%, 49%, 49.5%, or 50%) at least 55%, or at least 65%. In some aspects, the ORR is at least 47.2%. In some aspects, the ORR is about 47.2%. In some aspects, the ORR is 75% or greater. In some aspects, at least 1% of patients (e.g., at least 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of patients) have a complete response (CR) or a very good partial response (VGPR). In some aspects, the ORR is 40%-50%, and 10%-20% of patients have a CR or a VGPR. In some aspects, the ORR is at least 40%, and at least 20% of patients have a CR or a VGPR.

In some aspects, the average duration of response (DoR) for patients treated using the methods described herein is at least two months, e.g., at least three months, at least four months, at least five months, at least six months, at least seven months, at least eight months, at least nine months, at least ten months, at least eleven months, at least one year, or more than one year. In some aspects, the average DoR is at least four months. In some aspects, the average DoR is at least five months. In some aspects, the average DoR is at least seven months.

In some aspects, the six-month progression-free survival (PFS) rate for patients treated using the methods described herein is at least 10%, e.g., is at least 15%, 20%, 25%, 30%, 35%, 40%, 45%, 50%, 55%, 60%, 65%, 70%, 75%, 80%, 85%, 90%, 95%, 99%, or 100%. In some aspects, the six-month PFS rate is at least 25%. In some aspects, the six-month PFS rate is at least 40%. In some aspects, the six-month PFS rate is at least 55%.

### H. Methods of Administration

The methods may involve administering the bispecific anti-FcRH5/anti-CD3 antibody (and/or any additional therapeutic agent) by any suitable means, including parenteral, intrapulmonary, and intranasal, and, if desired for local treatment, intralesional administration. Parenteral infusions include intravenous, subcutaneous, intramuscular, intraarterial, and intraperitoneal administration routes. In some embodiments, the bispecific anti-FcRH5/anti-CD3 antibody is administered by intravenous infusion. In other instances, the bispecific anti-FcRH5/anti-CD3 antibody is administered subcutaneously.

In some instances, the bispecific anti-FcRH5/anti-CD3 antibody administered by intravenous injection exhibits a less toxic response (i.e., fewer unwanted effects) in a patient than the same bispecific anti-FcRH5/anti-CD3 antibody administered by subcutaneous injection, or vice versa.

In some aspects, the bispecific anti-FcRH5/anti-CD3 antibody is administered intravenously over 4 hours (±15 minutes), e.g., the first dose of the antibody is administered over 4 hours±15 minutes.

In some aspects, the first dose and the second dose of the antibody are administered intravenously with a median infusion time of less than four hours (e.g., less than three hours, less than two hours, or less than one hour) and further doses of the antibody are administered intravenously with a median infusion time of less than 120 minutes (e.g., less than 90 minutes, less than 60 minutes, or less than 30 minutes.

In some aspects, the first dose and the second dose of the antibody are administered intravenously with a median infusion time of less than three hours and further doses of the antibody are administered intravenously with a median infusion time of less than 90 minutes.

In some aspects, the first dose and the second dose of the antibody are administered intravenously with a median infusion time of less than three hours and further doses of the antibody are administered intravenously with a median infusion time of less than 60 minutes. In some aspects, the patient is hospitalized (e.g., hospitalized for 72 hours, 48 hours, 24 hours, or less than 24 hours) during one or more administrations of the anti-FcRH5/anti-CD3 antibody, e.g., hospitalized for the C1D1 (cycle 1, dose 1) or the C1D1 and the C1D2 (cycle 1, dose 2). In some aspects, the patient is hospitalized for 72 hours following administration of the C1D1 and the C1D2. In some aspects, the patient is hospitalized for 24 hours following administration of the C1D1 and the C1D2. In some aspects, the patient is not hospitalized following the administration of any dose of the anti-FcRH5/anti-CD3 antibody.

For all the methods described herein, the bispecific anti-FcRH5/anti-CD3 antibody would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The bispecific anti-FcRH5/anti-CD3 antibody need not be, but is optionally formulated with, one or more agents currently used to prevent or treat the disorder in question. The effective amount of such other agents depends on the amount of the bispecific anti-FcRH5/anti-CD3 antibody present in the formulation, the type of disorder or treatment, and other factors discussed above. The bispecific anti-FcRH5/anti-CD3 antibody may be suitably administered to the patient over a series of treatments.

Any of the doses disclosed herein may be administered SC. Any suitable approach for SC administration may be used, including injection (e.g., a bolus injection) or infusion. For example, the therapeutic agent (e.g., bispecific anti-FcRH5/anti-CD3 antibody) may be administered SC using a pump (e.g., a patch pump, a syringe pump (e.g., a syringe pump with an infusion set), or an infusion pump (e.g., an ambulatory infusion pump or a stationary infusion pump)), a pre-filled syringe, a pen injector, or an autoinjector.

For example, in any of the methods or uses disclosed herein, the therapeutic agent may be administered SC using a pump. In some examples, a pump may be used for patient or health care provider (HCP) convenience, an improved safety profile (e.g., in terms of a drug's mechanism of action or the risk of IV-related infection), and/or for a combination therapy. Any suitable pump may be used, e.g., a patch pump, a syringe pump (e.g., a syringe pump with an infusion set), an infusion pump (e.g., an ambulatory infusion pump or a stationary infusion pump), or an LVP. In particular examples, the therapeutic agent may be administered SC using a patch pump. In some examples, the pump (e.g., the patch pump) may be a wearable or on-body pump (e.g., a wearable or on-body patch pump), for example, an Enable ENFUSE® on-body infusor or a West SMARTDOSE® wearable injector (e.g., a West SMARTDOSE® 10 wearable injector). In other examples, the therapeutic agent may be administered SC using a syringe pump (e.g., a syringe pump with an infusion set).

Other exemplary devices suitable for SC delivery include: a syringe (including a pre-filled syringe); an injection device (e.g., the INJECT-EASE™ and GENJECT™ device); an infusion pump (such as e.g., Accu-Chek™); an injector pen (such as the GENPEN™); a needleless device (e.g., MEDDECTOR™ and BIOJECTOR™); an autoinjector, a subcutaneous patch delivery system, and the like.

In certain embodiments, the subcutaneous administration device is a prefilled syringe comprising a glass barrel, a plunger rod comprising a plunger stopper and a needle. In certain embodiments, the subcutaneous administration device further comprises a needle shield and optionally a needle shield device. In certain embodiments, the volume of formulation contained in the prefilled syringe is 0.3 mL, 1 mL, 1.5 mL, or 2.0 mL, in certain embodiments, the needle is a staked-in needle comprising a 3-bevel tip or a 5-bevel tip. In one embodiment, the subcutaneous administration device comprises a prefilled 1.0 mL low tungsten borosilicate glass (type I) syringe and a stainless steel 5-bevel 27 G ½ inch long thin-wall staked-in needle. In certain embodiments, the plunger rod comprises a rubber plunger stopper. In certain embodiments, the rubber plunger stopper comprises 4023/50 rubber and FLUROTEC® ethylene-tetrafluoroethylene (ETFE) coating. In some embodiments, the width (diameter, in particular outer diameter) of a needle for subcutaneous administration is typically between 25 gauge (G) and 31 G and is between ½ inch, long and % inch long. In some particular examples, the diameter, in particular the outer diameter, of a needle for subcutaneous administration is at least 28 G. Even more preferably, the diameter, in particular the outer diameter, of a needle or subcutaneous administration (e.g., injection) is at least 29 G, for example 29 G, 29½ G, 30 G, 30 5/16 G, or 31 G. In some further particular examples, the diameter, in particular the outer diameter, of a needle for subcutaneous administration is at least 30 G. The use of such needles having very small outer diameters is assumed to further modify the cytokine release, possibly by causing smaller lesions and/or by causing a slower administration (less volume released over the same time). Needle injection typically requires injection by positioning the needle at an angle within the range of 40° to 50°. In certain embodiments, the subcutaneous administration device comprises a rigid needle shield. In certain embodiments, the rigid needle shield comprises a rubber formulation having low zinc content. In one embodiment, the needle shield is rigid and comprises an elastomeric component, FM27/0, and rigid polypropylene shield. In certain embodiments the subcutaneous administration device comprises a needle safety device. Exemplary needle safety devices include, but are not limited to, Ultrasafe Passive® Needle Guard X100L (Safety Syringes, Inc.) and Rexam Safe n Sound™ (Rexam).

In some embodiments, administration with the bispecific anti-FcRH5/anti-CD3 antibody is used with, for example, a self-inject device, autoinjector device, or other device designed for self-administration. In certain embodiments, the bispecific anti-FcRH5/anti-CD3 antibody is administered using a subcutaneous administration device. Various self-inject devices and subcutaneous administration devices, including autoinjector devices, are known in the art and are commercially available. Exemplary devices include, but are not limited to, prefilled syringes (such as BD HYPAK SCF®, READYFILL™, and STERIFILL SCF™ from Becton Dickinson; CLEARSHOT™ copolymer prefilled syringes from Baxter; and Daikyo Seiko CRYSTAL ZENITH® prefilled syringes available from West Pharmaceutical Services); disposable pen injection devices such as BD Pen from Becton Dickinson; ultra-sharp and microneedle devices (such as INJECT-EASE™ and microinfuser devices from Becton Dickinson; and H-PATCH™ available from Valeritas) as well as needle-free injection devices (such as BIOJECTOR® and IJECT® available from Bioject; and SOF-SERTER® and patch devices available from Medtronic). Certain embodiments of subcutaneous administration devices are described further herein. Co-formulations or co-administrations with such self-inject devices or subcutaneous administration devices of the bispecific anti-FcRH5/anti-CD3 antibody with at least a second therapeutic compound are envisioned.

In some embodiments, administration with the bispecific anti-FcRH5/anti-CD3 antibody is in combination with soluble hyaluronidase glycoproteins (sHASEGPs), which has been shown to facilitate the subcutaneous injection of therapeutic antibodies; see WO2006/091871. It has been shown that the addition of such soluble hyaluronidase glycoproteins (either as a combined formulation or by co-administration) may facilitate the administration of therapeutic drug into the hypodermis. By rapidly depolymerizing hyaluronan HA in the extracellular space, sHASEGP can reduce the viscosity of the interstitium, thereby increasing hydraulic conductance and allowing for larger volumes to be administered safely and comfortably into the subcutaneous tissue. The increased hydraulic conductance induced by sHASEGP through reduced interstitial viscosity can allow for greater dispersion, potentially increasing the systemic bioavailability of SC administered therapeutic drug. In some embodiments, a hyaluronidase, such as rHuPH20, is included in the formulation, for example, in an amount from about 1,400 U/mL to about 1,600 U/mL (e.g., about 1,500 U/mL). Optionally, the device delivers 0.9 mL, 1.8 mL, or 3.6 mL of the formulation to a subject.

Hyaluronidase products of animal origin have been used clinically for over 60 years, primarily to increase the dispersion and absorption of other co-administered drugs and for hypodermoclysis (SC injection/infusion of fluid in large volume) (Frost G. I., “Recombinant human hyaluronidase (rHuPH20): an enabling platform for subcutaneous drug and fluid administration”, Expert Opinion on Drug Delivery, 2007; 4: 427-440). The details on the mechanism of action of hyaluronidases have been described in detail in the following publications: Duran-Reynolds F., “A spreading factor in certain snake venoms and its relation to their mode of action”, CR Soc Biol Paris, 1938; 69-81; Chain E., “A mucolytic enzyme in testes extracts”, Nature 1939; 977-978; Weissmann B., “The transglycosylative action of testicular hyaluronidase”, J. Biol. Chem., 1955; 216: 783-94; Tammi, R., Saamanen, A. M., Maibach, H. I., Tammi M., “Degradation of newly synthesized high molecular mass hyaluronan in the epidermal and dermal compartments of human skin in organ culture”, J. Invest. Dermatol. 1991: 97:126-130; Laurent, U. B. G., Dahl, L. B., Reed, R. K., “Catabolism of hyaluronan in rabbit skin takes place locally, in lymph nodes and liver”. Exp. Physiol. 1991; 76: 695-703; Laurent, T. C. and Fraser, J. R. E., “Degradation of Bioactive Substances: Physiology and Pathophysiology”, Henriksen. J. H. (Ed) CRC Press, Boca Raton, Fla.; 1991. pp. 249-265; Hams, E. N., et al., “Endocytic function, glycosaminoglycan specificity, and antibody sensitivity of the recombinant human 190-kDa hyaluronan receptor for endocytosis (HARE)”, J. Biol. Chem. 2004; 279:36201-36209; Frost, G. I., “Recombinant human hyaluronidase (rHuPH20): an enabling platform for subcutaneous drug and fluid administration”. Expert Opinion on Drug Delivery, 2007; 4: 427-440.

The bispecific anti-FcRH5/anti-CD3 antibody may be administered to patients subcutaneously into the subcutaneous tissue of the abdomen. The abdomen can be divided into 4 quadrants, and injection sites can be rotated as shown. Other sites for administering the cevostamab subcutaneously may include, but are not limited to, the outer area of the upper arm, the thoracic region, in particular the lower thoracic region, the abdominal wall, above or below the waist, the upper area of the buttock, just behind the hip bone and the thigh, in particular the front of the thigh. Preferred sites for administering the antibody subcutaneously include the abdominal wall, and the lower thoracic region. Within a treatment cycle, each single dose may be administered to essentially the same body site, e.g., the thigh or abdomen. Alternatively, each single dose within a treatment cycle may be administered to different body sites. The target area of administration can be the fat layer located between the dermis and underlying fascia.

### I. Anti-FcRH5/Anti-CD3 Bispecific Antibodies

The methods described herein include administering to a subject having a cancer (e.g., a multiple myeloma, e.g., an R/R multiple myeloma) a bispecific antibody that binds to FcRH5 and CD3 (i.e., a bispecific anti-FcRH5/anti-CD3 antibody).

In some instances, any of the methods described herein may include administering a bispecific antibody that includes an anti-FcRH5 arm having a first binding domain comprising at least one, two, three, four, five, or six hypervariable regions (HVRs) selected from (a) an HVR-H1 comprising the amino acid sequence of RFGVH (SEQ ID NO: 1); (b) an HVR-H2 comprising the amino acid sequence of VIWRGGSTDYNAAFVS (SEQ ID NO: 2); (c) an HVR-H3 comprising the amino acid sequence of HYYGSSDYALDN (SEQ ID NO:3); (d) an HVR-L1 comprising the amino acid sequence of KASQDVRNLVV (SEQ ID NO: 4); (e) an HVR-L2 comprising the amino acid sequence of SGSYRYS (SEQ ID NO: 5); and (f) an HVR-L3 comprising the amino acid sequence of QQHYSPPYT (SEQ ID NO: 6). In some instances, the bispecific anti-FcRH5/anti-CD3 antibody comprises at least one (e.g., 1, 2, 3, or 4) of the heavy chain framework regions FR-H1, FR-H2, FR-H3, and FR-H4 comprising the sequences of SEQ ID NOs: 17-20, respectively, and/or at least one (e.g., 1, 2, 3, or 4) of the light chain framework regions FR-L1, FR-L2, FR-L3, and FR-L4 comprising the sequences of SEQ ID NOs: 21-24, respectively.

In some instances, any of the methods described herein may include administering a bispecific antibody that includes an anti-FcRH5 arm having a first binding domain comprising the following six HVRs: (a) an HVR-H1 comprising the amino acid sequence of RFGVH (SEQ ID NO: 1); (b) an HVR-H2 comprising the amino acid sequence of VIWRGGSTDYNAAFVS (SEQ ID NO: 2); (c) an HVR-H3 comprising the amino acid sequence of HYYGSSDYALDN (SEQ ID NO:3); (d) an HVR-L1 comprising the amino acid sequence of KASQDVRNLVV (SEQ ID NO: 4); (e) an HVR-L2 comprising the amino acid sequence of SGSYRYS (SEQ ID NO: 5); and (f) an HVR-L3 comprising the amino acid sequence of QQHYSPPYT (SEQ ID NO: 6). In some instances, the bispecific anti-FcRH5/anti-CD3 antibody comprises at least one (e.g., 1, 2, 3, or 4) of the heavy chain framework regions FR-H1, FR-H2, FR-H3, and FR-H4 comprising the sequences of SEQ ID NOs: 17-20, respectively, and/or at least one (e.g., 1, 2, 3, or 4) of the light chain framework regions FR-1, FR-L2, FR-L3, and FR-L4 comprising the sequences of SEQ ID NOs: 21-24, respectively.

In some instances, the bispecific antibody comprises an anti-FcRH5 arm comprising a first binding domain comprising (a) a heavy chain variable (VH) domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 7; (b) a light chain variable (VL) domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 8; or (c) a VH domain as in (a) and a VL domain as in (b). Accordingly, in some instances, the first binding domain comprises a VH domain comprising an amino acid sequence of SEQ ID NO: 7 and a VL domain comprising an amino acid sequence of SEQ ID NO:8.

In some instances, any of the methods described herein may include administering a bispecific anti-FcRH5/anti-CD3 antibody that includes an anti-CD3 arm having a second binding domain comprising at least one, two, three, four, five, or six HVRs selected from (a) an HVR-H1 comprising the amino acid sequence of SYYIH (SEQ ID NO: 9); (b) an HVR-H2 comprising the amino acid sequence of WIYPENDNTKYNEKFKD (SEQ ID NO: 10); (c) an HVR-H3 comprising the amino acid sequence of DGYSRYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WTSTRKS (SEQ ID NO: 13); and (f) an HVR-L3 comprising the amino acid sequence of KQSFILRT (SEQ ID NO: 14). In some instances, the anti-FcRH5/anti-CD3 bispecific antibody comprises at least one (e.g., 1, 2, 3, or 4) of heavy chain framework regions FR-H1, FR-H2, FR-H3, and FR-H4 comprising the sequences of SEQ ID NOs: 25-28, respectively, and/or at least one (e.g., 1, 2, 3, or 4) of the light chain framework regions FR-L1, FR-L2, FR-L3, and FR-L4 comprising the sequences of SEQ ID NOs: 29-32, respectively.

In some instances, any of the methods described herein may include administering a bispecific anti-FcRH5/anti-CD3 antibody that includes an anti-CD3 arm having a second binding domain comprising the following six HVRs: (a) an HVR-H1 comprising the amino acid sequence of SYYIH (SEQ ID NO: 9); (b) an HVR-H2 comprising the amino acid sequence of WIYPENDNTKYNEKFKD (SEQ ID NO: 10); (c) an HVR-H3 comprising the amino acid sequence of DGYSRYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WTSTRKS (SEQ ID NO: 13); and (f) an HVR-L3 comprising the amino acid sequence of KQSFILRT (SEQ ID NO: 14). In some instances, the anti-FcRH5/anti-CD3 bispecific antibody comprises at least one (e.g., 1, 2, 3, or 4) of heavy chain framework regions FR-H1, FR-H2, FR-H3, and FR-H4 comprising the sequences of SEQ ID NOs: 25-28, respectively, and/or at least one (e.g., 1, 2, 3, or 4) of the light chain framework regions FR-L1, FR-L2, FR-L3, and FR-L4 comprising the sequences of SEQ ID NOs: 29-32, respectively.

In some instances, the bispecific antibody comprises an anti-CD3 arm comprising a second binding domain comprising (a) a VH domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 15; (b) a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 16; or (c) a VH domain as in (a) and a VL domain as in (b). Accordingly, in some instances, the second binding domain comprises a VH domain comprising an amino acid sequence of SEQ ID NO: 15 and a VL domain comprising an amino acid sequence of SEQ ID NO: 16.

In some instances, any of the methods described herein may include administering a bispecific antibody that includes (1) an anti-FcRH5 arm having a first binding domain comprising at least one, two, three, four, five, or six HVRs selected from (a) an HVR-H1 comprising the amino acid sequence of RFGVH (SEQ ID NO: 1); (b) an HVR-H2 comprising the amino acid sequence of VIWRGGSTDYNAAFVS (SEQ ID NO: 2); (c) an HVR-H3 comprising the amino acid sequence of HYYGSSDYALDN (SEQ ID NO:3); (d) an HVR-L1 comprising the amino acid sequence of KASQDVRNLVV (SEQ ID NO: 4); (e) an HVR-L2 comprising the amino acid sequence of SGSYRYS (SEQ ID NO: 5); and (f) an HVR-L3 comprising the amino acid sequence of QQHYSPPYT (SEQ ID NO: 6) and (2) an anti-CD3 arm having a second binding domain comprising at least one, two, three, four, five, or six HVRs selected from (a) an HVR-H1 comprising the amino acid sequence of SYYIH (SEQ ID NO: 9); (b) an HVR-H2 comprising the amino acid sequence of WIYPENDNTKYNEKFKD (SEQ ID NO: 10); (c) an HVR-H3 comprising the amino acid sequence of DGYSRYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WTSTRKS (SEQ ID NO: 13); and (f) an HVR-L3 comprising the amino acid sequence of KQSFILRT (SEQ ID NO: 14).

In some instances, any of the methods described herein may include administering a bispecific antibody that includes (1) an anti-FcRH5 arm having a first binding domain comprising the following six HVRs: (a) an HVR-H1 comprising the amino acid sequence of RFGVH (SEQ ID NO: 1); (b) an HVR-H2 comprising the amino acid sequence of VIWRGGSTDYNAAFVS (SEQ ID NO: 2); (c) an HVR-H3 comprising the amino acid sequence of HYYGSSDYALDN (SEQ ID NO:3); (d) an HVR-L1 comprising the amino acid sequence of KASQDVRNLVV (SEQ ID NO: 4); (e) an HVR-L2 comprising the amino acid sequence of SGSYRYS (SEQ ID NO: 5); and (f) an HVR-L3 comprising the amino acid sequence of QQHYSPPYT (SEQ ID NO: 6) and (2) an anti-CD3 arm having a second binding domain comprising the following six HVRs: (a) an HVR-H1 comprising the amino acid sequence of SYYIH (SEQ ID NO: 9); (b) an HVR-H2 comprising the amino acid sequence of WIYPENDNTKYNEKFKD (SEQ ID NO: 10); (c) an HVR-H3 comprising the amino acid sequence of DGYSRYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WTSTRKS (SEQ ID NO: 13); and (f) an HVR-L3 comprising the amino acid sequence of KQSFILRT (SEQ ID NO: 14).

In some instances, the anti-FcRH5/anti-CD3 bispecific antibody comprises (1) at least one (e.g., 1, 2, 3, or 4) of heavy chain framework regions FR-H1, FR-H2, FR-H3, and FR-H4 comprising the sequences of SEQ ID NOs: 17-20, respectively, and/or at least one (e.g., 1, 2, 3, or 4) of the light chain framework regions FR-L1, FR-L2, FR-L3, and FR-L4 comprising the sequences of SEQ ID NOs: 21-24, respectively, and (2) at least one (e.g., 1, 2, 3, or 4) of heavy chain framework regions FR-H1, FR-H2, FR-H3, and FR-H4 comprising the sequences of SEQ ID NOs: 25-28, respectively, and/or at least one (e.g., 1, 2, 3, or 4) of the light chain framework regions FR-L1, FR-L2, FR-L3, and FR-L4 comprising the sequences of SEQ ID NOs: 29-32, respectively. In some instances, the anti-FcRH5/anti-CD3 bispecific antibody comprises (1) all four of heavy chain framework regions FR-H1, FR-H2, FR-H3, and FR-H4 comprising the sequences of SEQ ID NOs: 17-20, respectively, and/or all four of the light chain framework regions FR-L1, FR-L2, FR-L3, and FR-L4 comprising the sequences of SEQ ID NOs: 21-24, respectively, and (2) all four of heavy chain framework regions FR-H1, FR-H2, FR-H3, and FR-H4 comprising the sequences of SEQ ID NOs: 25-28, respectively, and/or all four (e.g., 1, 2, 3, or 4) of the light chain framework regions FR-L1, FR-L2, FR-L3, and FR-L4 comprising the sequences of SEQ ID NOs: 29-32, respectively.

In some instances, the anti-FcRH5/anti-CD3 bispecific antibody comprises (1) an anti-FcRH5 arm comprising a first binding domain comprising (a) a VH domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 7; (b) a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 8; or (c) a VH domain as in (a) and a VL domain as in (b), and (2) an anti-CD3 arm comprising a second binding domain comprising (a) a VH domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 15; (b) a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 16; or (c) a VH domain as in (a) and a VL domain as in (b). In some instances, the anti-FcRH5/anti-CD3 bispecific antibody comprises (1) a first binding domain comprising a VH domain comprising an amino acid sequence of SEQ ID NO: 7 and a VL domain comprising an amino acid sequence of SEQ ID NO: 8 and (2) a second binding domain comprising a VH domain comprising an amino acid sequence of SEQ ID NO: 15 and a VL domain comprising an amino acid sequence of SEQ ID NO: 16.

In some instances, the anti-FcRH5/anti-CD3 bispecific antibody comprises an anti-FcRH5 arm comprising a heavy chain polypeptide (H1) and a light chain polypeptide (L1), wherein (a) H1 comprises an amino acid sequence having at least 90% sequence identity (e.g., at least 91%, 92%, 93%, 94%, 95%, 96%, 97%, 98%, or 99% sequence identity) to, or the sequence of, SEQ ID NO: 35 and/or (b) L1 comprises an amino acid sequence having at least 90% sequence identity (e.g., at least 91%, 92%, 93%, 94%, 95%, 96%, 97%, 98%, or 99% sequence identity) to, or the sequence of, SEQ ID NO: 36.

In some instances, the anti-FcRH5/anti-CD3 bispecific antibody comprises an anti-FcRH5 arm comprising a heavy chain polypeptide (H1) and a light chain polypeptide (L1), wherein (a) H1 comprises the amino acid sequence of SEQ ID NO: 35 and/or (b) L1 comprises the amino acid sequence of SEQ ID NO: 36.

In some instances, the anti-FcRH5/anti-CD3 bispecific antibody comprises an anti-CD3 arm comprising a heavy chain polypeptide (H2) and a light chain polypeptide (L2), wherein (a) H2 comprises an amino acid sequence having at least 90% sequence identity (e.g., at least 91%, 92%, 93%, 94%, 95%, 96%, 97%, 98%, or 99% sequence identity) to, or the sequence of, SEQ ID NO: 37 and/or (b) L2 comprises an amino acid sequence having at least 90% sequence identity (e.g., at least 91%, 92%, 93%, 94%, 95%, 96%, 97%, 98%, or 99% sequence identity) to, or the sequence of, SEQ ID NO: 38.

In some instances, the anti-FcRH5/anti-CD3 bispecific antibody comprises an anti-CD3 arm comprising a heavy chain polypeptide (H2) and a light chain polypeptide (L2), wherein (a) H2 comprises the amino acid sequence of SEQ ID NO: 37; and (b) L2 comprises the amino acid sequence of SEQ ID NO: 38.

In some instances, the anti-FcRH5/anti-CD3 bispecific antibody comprises an anti-FcRH5 arm comprising a heavy chain polypeptide (H1) and a light chain polypeptide (L1) and an anti-CD3 arm comprising a heavy chain polypeptide (H2) and a light chain polypeptide (L2), and wherein (a) H1 comprises an amino acid sequence having at least 90% sequence identity (e.g., at least 91%, 92%, 93%, 94%, 95%, 96%, 97%, 98%, or 99% sequence identity) to, or the sequence of, SEQ ID NO: 35; (b) L1 comprises an amino acid sequence having at least 90% sequence identity (e.g., at least 91%, 92%, 93%, 94%, 95%, 96%, 97%, 98%, or 99% sequence identity) to, or the sequence of, SEQ ID NO: 36; (c) H2 comprises an amino acid sequence having at least 90% sequence identity (e.g., at least 91%, 92%, 93%, 94%, 95%, 96%, 97%, 98%, or 99% sequence identity) to, or the sequence of, SEQ ID NO: 37; and (d) L2 comprises an amino acid sequence having at least 90% sequence identity (e.g., at least 91%, 92%, 93%, 94%, 95%, 96%, 97%, 98%, or 99% sequence identity) to, or the sequence of, SEQ ID NO: 38.

In some instances, the anti-FcRH5/anti-CD3 bispecific antibody comprises an anti-FcRH5 arm comprising a heavy chain polypeptide (H1) and a light chain polypeptide (L1) and an anti-CD3 arm comprising a heavy chain polypeptide (H2) and a light chain polypeptide (L2), and wherein (a) H1 comprises the amino acid sequence of SEQ ID NO: 35; (b) L1 comprises the amino acid sequence of SEQ ID NO: 36; (c) H2 comprises the amino acid sequence of SEQ ID NO: 37; and (d) L2 comprises the amino acid sequence of SEQ ID NO: 38.

In some instances, the anti-FcRH5/anti-CD3 bispecific antibody is cevostamab.

In some instances, the anti-FcRH5/anti-CD3 bispecific antibody according to any of the above embodiments described above may incorporate any of the features, singly or in combination, as described in Sections 1-7 below.

### 1. Antibody Affinity

In certain embodiments, an antibody provided herein has a dissociation constant (K_{D}) of ≤1 μM, ≤250 nM, ≤100 nM, ≤15 nM, ≤10 nM, ≤6 nM, ≤4 nM, ≤2 nM, ≤1 nM, ≤0.1 nM, ≤0.01 nM, or ≤0.001 nM (e.g. 10⁻⁸ M or less, e.g., from 10⁻⁸ M to 10⁻¹³ M, e.g., from 10⁻⁹ M to 10⁻¹³ M).

In one embodiment, K_{D} is measured by a radiolabeled antigen binding assay (RIA). In one embodiment, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, K_{D} is measured using a BIACORE® surface plasmon resonance assay. For example, an assay using a BIACORE®-2000 or a BIACORE®-3000 (BIAcore, Inc., Piscataway, NJ) is performed at 37° C. with immobilized antigen CM5 chips at ˜10 response units (RU). In one embodiment, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 37° C. at a flow rate of approximately 25 μl/min. Association rates (kₒₙ, or kₐ) and dissociation rates (k_{off}, or k_{d}) are calculated using a simple one-to-one Langmuir binding model (BIACORE® Evaluation Software version 3.2) by simultaneously fitting the association and dissociation sensorgrams. The equilibrium dissociation constant (K_{D}) is calculated as the ratio k_{off}/kₒₙ. See, for example, Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 37° C. of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLM-AMINCO™ spectrophotometer (ThermoSpectronic) with a stirred cuvette.

### 2. Antibody Fragments

In certain embodiments, an antibody provided herein (e.g., an anti-FcRH5/anti-CD3 TDB) is an antibody fragment that binds FcRH5 and CD3. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al. Nat. Med. 9:129-134 (2003); and Hollinger et al. Proc. Natl. Acad. Sci. USA 90: 6444-6448 (1993). Triabodies and tetrabodies are also described in Hudson et al. Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, M A; see, e.g., U.S. Pat. No. 6,248,516 B1).

Antibody fragments can be made by various techniques, including but not limited to proteolytic digestion of an intact antibody as well as production by recombinant host cells (e.g., E. coli or phage), as described herein.

### 3. Chimeric and Humanized Antibodies

In certain embodiments, an antibody provided herein (e.g., an anti-FcRH5/anti-CD3 TDB) is a chimeric antibody. Certain chimeric antibodies are described, e.g., in U.S. Pat. No. 4,816,567; and Morrison et al. Proc. Nat. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs (or portions thereof), for example, are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 4. Human Antibodies

In certain embodiments, an antibody provided herein (e.g., an anti-FcRH5/anti-CD3 TDB) is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991)). Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

### 5. Multispecific Antibodies

In any one of the above aspects, an anti-FcRH5/anti-CD3 antibody provided herein is a multispecific antibody, for example, a bispecific antibody. Multispecific antibodies are antibodies (e.g., monoclonal antibodies) that have binding specificities for at least two different sites, e.g., antibodies having binding specificities for an immune effector cell and for a cell surface antigen (e.g., a tumor antigen, e.g., FcRH5) on a target cell other than an immune effector cell. In some aspects, one of the binding specificities is for FcRH5 and the other is for CD3.

In some aspects, the cell surface antigen may be expressed in low copy number on the target cell. For example, in some aspects, the cell surface antigen is expressed or present at less than 35,000 copies per target cell. In some embodiments, the low copy number cell surface antigen is present between 100 and 35,000 copies per target cell; between 100 and 30,000 copies per target cell; between 100 and 25,000 copies per target cell; between 100 and 20,000 copies per target cell; between 100 and 15,000 copies per target cell; between 100 and 10,000 copies per target cell; between 100 and 5,000 copies per target cell; between 100 and 2,000 copies per target cell; between 100 and 1,000 copies per target cell; or between 100 and 500 copies per target cell. Copy number of the cell surface antigen can be determined, for example, using a standard Scatchard plot.

In some embodiments, a bispecific antibody may be used to localize a cytotoxic agent to a cell that expresses a tumor antigen, e.g., FcRH5. Bispecific antibodies may be prepared as full-length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). “Knob-in-hole” engineering of multispecific antibodies may be utilized to generate a first arm containing a knob and a second arm containing the hole into which the knob of the first arm may bind. The knob of the multispecific antibodies of the invention may be an anti-CD3 arm in one embodiment. Alternatively, the knob of the multispecific antibodies of the invention may be an anti-target/antigen arm in one embodiment. The hole of the multispecific antibodies of the invention may be an anti-CD3 arm in one embodiment. Alternatively, the hole of the multispecific antibodies of the invention may be an anti-target/antigen arm in one embodiment.

Multispecific antibodies may also be engineered using immunoglobulin crossover (also known as Fab domain exchange or CrossMab format) technology (see, e.g., WO2009/080253; Schaefer et al., Proc. Natl. Acad. Sci. USA, 108:11187-11192 (2011)). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g., US 2006/0025576A1).

The antibodies, or antibody fragments thereof, may also include a “Dual Acting FAb” or “DAF” comprising an antigen binding site that binds to CD3 as well as another, different antigen (e.g., a second biological molecule) (see, e.g., US 2008/0069820).

### 6. Antibody Variants

In some aspects, amino acid sequence variants of the antibodies described herein, e.g., bispecific anti-FcRH5/anti-CD3 antibodies, are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, for example, antigen-binding.

a. Substitution, Insertion, and Deletion Variants

In certain embodiments, antibody variants having one or more amino acid substitutions are provided. Sites of interest for substitutional mutagenesis include the CDRs and FRs. Conservative substitutions are shown in Table 4 under the heading of “preferred substitutions.” More substantial changes are provided in Table 4 under the heading of “exemplary substitutions,” and as further described below in reference to amino acid side chain classes. Amino acid substitutions may be introduced into an antibody of interest and the products screened for a desired activity, for example, retained/improved antigen binding, decreased immunogenicity, or improved ADCC or CDC.

TABLE 4
Exemplary and Preferred Amino Acid Substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | lle; Leu; Met; Phe; Ala; Norleucine | Leu |

Amino acids may be grouped according to common side-chain properties:
(1) hydrophobic: Norleucine, Met, Ala, Val, Leu, lie;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more CDR residues are mutated and the variant antibodies displayed on phage and screened for a particular biological activity (e.g. binding affinity).

Alterations (e.g., substitutions) may be made in CDRs, e.g., to improve antibody affinity. Such alterations may be made in CDR “hotspots,” i.e., residues encoded by codons that undergo mutation at high frequency during the somatic maturation process (see, e.g., Chowdhury, Methods Mol. Biol. 207:179-196 (2008)), and/or residues that contact an antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001)). In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. Such alterations may, for example, be outside of antigen contacting residues in the CDRs. In certain embodiments of the variant VH and VL sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

### B. Glycosylation Variants

In certain embodiments, antibodies disclosed herein, e.g., bispecific anti-FcRH5/anti-CD3 antibodies, can be altered to increase or decrease the extent to which the antibody is glycosylated. Addition or deletion of glycosylation sites to anti-FcRH5 antibody of the invention may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants, e.g., bispecific anti-FcRH5/anti-CD3 antibody variants, are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibody variants, e.g., bispecific anti-FcRH5/anti-CD3 antibody variants, are further provided with bisected oligosaccharides, for example, in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

c. Fc Region Variants

In certain embodiments, one or more amino acid modifications may be introduced into the Fc region of an antibody disclosed herein, e.g., a bispecific anti-FcRH5/anti-CD3 antibody, thereby generating an Fc region variant (see e.g., US 2012/0251531). The Fc region variant may comprise a human Fc region sequence (e.g., a human IgG1, IgG2, IgG3 or IgG4 Fc region) comprising an amino acid modification (e.g., a substitution) at one or more amino acid positions.

In certain embodiments, the invention contemplates an antibody variant, e.g., a bispecific anti-FcRH5/anti-CD3 antibody variant, that possesses some but not all effector functions, which make it a desirable candidate for applications in which the half-life of the antibody in vivo is important, yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express Fc(RIII only, whereas monocytes express Fc(RI, Fc(RII and Fc(RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al. J. Immunol. Methods 202:163 (1996); Cragg, M. S. et al. Blood. 101:1045-1052 (2003); and Cragg, M. S. and M. J. Glennie Blood. 103:2738-2743 (2004)). FcRn binding and in vivo clearance/half-life determinations can also be performed using methods known in the art (see, e.g., Petkova, S. B. et al. Int'l. Immunol. 18(12):1759-1769 (2006)).

Antibodies with reduced effector function include those with substitution of one or more of Fc region residues 238, 265, 269, 270, 297, 327 and 329 (U.S. Pat. Nos. 6,737,056 and 8,219,149). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. Nos. 7,332,581 and 8,219,149).

In certain embodiments, the proline at position 329 of a wild-type human Fc region in the antibody is substituted with glycine or arginine or an amino acid residue large enough to destroy the proline sandwich within the Fc/Fcγ receptor interface that is formed between the proline 329 of the Fc and tryptophan residues Trp 87 and Trp 110 of FcγRIII (Sondermann et al. Nature. 406, 267-273, 2000). In certain embodiments, the antibody comprises at least one further amino acid substitution. In one embodiment, the further amino acid substitution is S228P, E233P, L234A, L235A, L235E, N297A, N297D, or P331S, and still in another embodiment the at least one further amino acid substitution is L234A and L235A of the human IgG1 Fc region or S228P and L235E of the human IgG4 Fc region (see e.g., US 2012/0251531), and still in another embodiment the at least one further amino acid substitution is L234A and L235A and P329G of the human IgG1 Fc region.

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

In some aspects, the antibody, e.g., the anti-FcRH5 and/or anti-CD3 antibody (e.g., bispecific anti-FcRH5 antibody) comprises an Fc region comprising an N297G mutation (EU numbering). In some aspects, the anti-FcRH5 arm of the bispecific anti-FcRH5 antibody comprises a N297G mutation and/or the anti-CD3 arm of the bispecific anti-FcRH5 antibody comprises an Fc region comprising an N297G mutation.

In some embodiments, the anti-FcRH5 antibody comprising the N297G mutation comprises an anti-FcRH5 arm comprising a first binding domain comprising the following six HVRs (a) an HVR-H1 comprising the amino acid sequence of SEQ ID NO: 1; (b) an HVR-H2 comprising the amino acid sequence of SEQ ID NO: 2; (c) an HVR-H3 comprising the amino acid sequence of SEQ ID NO: 3; (d) an HVR-L1 comprising the amino acid sequence of SEQ ID NO: 4; (e) an HVR-L2 comprising the amino acid sequence of SEQ ID NO: 5; and (f) an HVR-L3 comprising the amino acid sequence of SEQ ID NO: 6; and an anti-CD3 arm comprising an N297G mutation. In some embodiments, the anti-CD3 arm comprising the N297G mutation comprises the following six HVRs: (a) an HVR-H1 comprising the amino acid sequence of SEQ ID NO: 9; (b) an HVR-H2 comprising the amino acid sequence of SEQ ID NO: 10; (c) an HVR-H3 comprising the amino acid sequence of SEQ ID NO: 11; (d) an HVR-L1 comprising the amino acid sequence of SEQ ID NO: 12; (e) an HVR-L2 comprising the amino acid sequence of SEQ ID NO: 13; and (f) an HVR-L3 comprising the amino acid sequence of SEQ ID NO: 14.

In some embodiments, the anti-FcRH5 antibody comprising the N297G mutation comprises an anti-FcRH5 arm comprising a first binding domain comprising (a) a VH domain comprising an amino acid sequence of SEQ ID NO: 7 and (b) a VL domain comprising an amino acid sequence of SEQ ID NO: 8, and an anti-CD3 arm comprising an N297G mutation. In some embodiments, the anti-CD3 arm comprising the N297G mutation comprises comprising (a) a VH domain comprising an amino acid sequence of SEQ ID NO: 15 and (b) a VL domain comprising an amino acid sequence of SEQ ID NO: 16.

In some embodiments, the anti-FcRH5 antibody comprising the N297G mutation comprises one or more heavy chain constant domains, wherein the one or more heavy chain constant domains are selected from a first CH1 (CH1₁) domain, a first CH2 (CH2₁) domain, a first CH3 (CH3₁) domain, a second CH1 (CH1₂) domain, second CH2 (CH2₂) domain, and a second CH3 (CH3₂) domain. In some aspects, at least one of the one or more heavy chain constant domains is paired with another heavy chain constant domain. In some aspects, the CH3₁ and CH3₂ domains each comprise a protuberance or cavity, and wherein the protuberance or cavity in the CH3₁ domain is positionable in the cavity or protuberance, respectively, in the CH3₂ domain. In some aspects, the CH3₁ and CH3₂ domains meet at an interface between said protuberance and cavity. In some aspects, the CH2₁ and CH2₂ domains each comprise a protuberance or cavity, and wherein the protuberance or cavity in the CH2₁ domain is positionable in the cavity or protuberance, respectively, in the CH2₂ domain. In other instances, the CH2₁ and CH2₂ domains meet at an interface between said protuberance and cavity. In some aspects, the anti-FcRH5 antibody is an IgG1 antibody.

In some embodiments, the anti-FcRH5 antibody comprising the N297G mutation comprises an anti-FcRH5 arm comprising a first binding domain comprising (a) a VH domain comprising the amino acid sequence of SEQ ID NO: 7 and (b) a VL domain comprising the amino acid sequence of SEQ ID NO: 8, and an anti-CD3 arm, wherein (a) the anti-FcRH5 arm comprises T366S, L368A, Y407V, and N297G amino acid substitution mutations (EU numbering) and (b) the anti-CD3 arm comprises T366W and N297G substitution mutations (EU numbering). In some embodiments, the anti-CD3 arm comprising the T366W and N297G mutations comprises comprising (a) a VH domain comprising an amino acid sequence of SEQ ID NO: 15 and (b) a VL domain comprising an amino acid sequence of SEQ ID NO: 16.

In other embodiments, the anti-FcRH5 antibody comprising the N297G mutation comprises an anti-FcRH5 arm comprising a first binding domain comprising (a) a VH domain comprising an amino acid sequence of SEQ ID NO: 7 and (b) a VL domain comprising an amino acid sequence of SEQ ID NO: 8, and an anti-CD3 arm, wherein (a) the anti-FcRH5 arm comprises T366W and N297G substitution mutations (EU numbering) and (b) the anti-CD3 arm comprises T366S, L368A, Y407V, and N297G mutations (EU numbering). In some embodiments, the anti-CD3 arm comprising the N297G mutation comprises comprising (a) a VH domain comprising an amino acid sequence of SEQ ID NO: 15 and (b) a VL domain comprising an amino acid sequence of SEQ ID NO: 16.

d. Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, for example, in U.S. Pat. No. 7,521,541.

e. Antibody Derivatives

In certain embodiments, an antibody provided herein, e.g., a bispecific anti-FcRH5/anti-CD3 antibody provided herein, may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

### 7. Charged Regions

In some aspects, the binding domain that binds FcRH5 or CD3 comprises a VH1 comprising a charged region (CR₁) and a VL1 comprising a charged region (CR₂), wherein the CR₁ in the VH1 forms a charge pair with the CR₂ in the VL1. In some aspects, the CR₁ comprises a basic amino acid residue and the CR₂ comprises an acidic amino acid residue. In some aspects, the CR₁ comprises a Q39K substitution mutation (Kabat numbering). In some aspects, the CR₁ consists of the Q39K substitution mutation. In some aspects, the CR₂ comprises a Q38E substitution mutation (Kabat numbering). In some aspects, the CR₂ consists of the Q38E substitution mutation. In some aspects, the second binding domain that binds CD3 comprises a VH2 comprising a charged region (CR₃) and a VL2 comprising a charged region (CR₄), wherein the CR₄ in the VL2 forms a charge pair with the CR₃ in the VH2. In some aspects, the CR₄ comprises a basic amino acid residue and the CR₃ comprises an acidic amino acid residue. In some aspects, the CR₄ comprises a Q38K substitution mutation (Kabat numbering). In some aspects, the CR₄ consists of the Q38K substitution mutation. In some aspects, the CR₃ comprises a Q39E substitution mutation (Kabat numbering). In some aspects, the CR₃ consists of the Q39E substitution mutation. In some aspects, the VL1 domain is linked to a light chain constant domain (CL1) domain and the VH1 is linked to a first heavy chain constant domain (CH1), wherein the CL1 comprises a charged region (CR₅) and the CH1 comprises a charged region (CR₆), and wherein the CR₅ in the CL1 forms a charge pair with the CR₆ in the CH1₁. In some aspects, the CR₅ comprises a basic amino acid residue and the CR₆ comprises an acidic residue. In some aspects, the CR₅ comprises a V133K substitution mutation (EU numbering). In some aspects, the CR₅ consists of the V133K substitution mutation. In some aspects, the CR₆ comprises a S183E substitution mutation (EU numbering). In some aspects, the CR₆ consists of the S183E substitution mutation.

In other aspects, the VL2 domain is linked to a CL domain (CL2) and the VH2 is linked to a CH1 domain (CH1₂), wherein the CL2 comprises a charged region (CR₇) and the CH1₂ comprises a charged region (CR₈), and wherein the CR₈ in the CH1₂ forms a charge pair with the CR₇ in the CL2. In some aspects, the CR₈ comprises a basic amino acid residue and the CR₇ comprises an acidic amino acid residue. In some aspects, the CR₈ comprises a S183K substitution mutation (EU numbering). In some aspects, the CR₈ consists of the S183K substitution mutation. In some aspects, the CR₇ comprises a V133E substitution mutation (EU numbering). In some aspects, the CR₇ consists of the V133E substitution mutation.

In other aspects, the VL2 domain is linked to a CL domain (CL2) and the VH2 is linked to a CH1 domain (CH1₂), wherein (a) the CL2 comprises one or more mutations at amino acid residues F116, L135, S174, S176, and/or T178 (EU numbering) and (b) the CH1₂ comprises one or more mutations at amino acid residues A141, F170, S181, S183, and/or V185 (EU numbering). In some aspects, the CL2 comprises one or more of the following substitution mutations: F116A, L135V, S174A, S176F, and/or T178V. In some aspects, the CL2 comprises the following substitution mutations: F116A, L135V, S174A, S176F, and T178V. In some aspects, the CH1₂ comprises one or more of the following substitution mutations: A141I, F170S, S181M, S183A, and/or V185A. In some aspects, the CH1₂ comprises the following substitution mutations: A141I, F170S, S181M, S183A, and V185A.

In other aspects, the binding domain that binds FcRH5 or CD3 comprises a VH domain (VH1) comprising a charged region (CR₁) and a VL domain (VL1) comprising a charged region (CR₂), wherein the CR₂ in the VL₁ forms a charge pair with the CR₁ in the VH1. In some aspects, the CR₂ comprises a basic amino acid residue and the CR₁ comprises an acidic amino acid residue. In some aspects, the CR₂ comprises a Q38K substitution mutation (Kabat numbering). In some aspects, the CR₂ consists of the Q38K substitution mutation. In some aspects, the CR₁ comprises a Q39E substitution mutation (Kabat numbering). In some aspects, the CR₁ consists of the Q39E substitution mutation. In some aspects, the second binding domain that binds CD3 comprises a VH domain (VH2) comprising a charged region (CR₃) and a VL domain (VL2) comprising a charged region (CR₄), wherein the CR₃ in the VH2 forms a charge pair with the CR₄ in the VL2. In some aspects, the CR₃ comprises a basic amino acid residue and the CR₄ comprises an acidic amino acid residue. In some aspects, the CR₃ comprises a Q39K substitution mutation (Kabat numbering). In some aspects, the CR₃ consists of the Q39K substitution mutation. In some aspects, the CR₄ comprises a Q38E substitution mutation (Kabat numbering). In some aspects, the CR₄ consists of the Q38E substitution mutation. In some aspects, the VL1 domain is linked to a light chain constant domain (CL1) and the VH1 is linked to a first heavy chain constant domain (CH1₁), wherein the CL1 comprises a charged region (CR₅) and the CH1 comprises a charged region (CR₆), and wherein the CR₆ in the CH1₁ forms a charge pair with the CR₅ in the CL1. In some aspects, the CR₆ comprises a basic amino acid residue and the CR₅ comprises an acidic amino acid residue. In some aspects, the CR₆ comprises a S183K substitution mutation (EU numbering). In some aspects, the CR₆ consists of the S183K substitution mutation. In some aspects, the CR₅ comprises a V133E substitution mutation (EU numbering). In some aspects, the CR₅ consists of the V133E substitution mutation.

In other aspects, the VL2 domain is linked to a CL domain (CL2) and the VH2 is linked to a CH1 domain (CH1₂), wherein the CL2 comprises a charged region (CR₇) and the CH1₂ comprises a charged region (CR₈), and wherein the CR₇ in the CL2 forms a charged pair with the CR₈ in the CH1₂. In some aspects, the CR₇ comprises a basic amino acid residue and the CR₈ comprises an acidic residue. In some aspects, the CR₇ comprises a V133K substitution mutation (EU numbering). In some aspects, the CR₇ consists of the V133K substitution mutation. In some aspects, the CR₈ comprises a S183E substitution mutation (EU numbering). In some aspects, the CR₈ consists of the S183E substitution mutation.

In other aspects, the VL2 domain is linked to a CL domain (CL2) and the VH2 is linked to a CH1 domain (CH1₂), wherein (a) the CL2 comprises one or more mutations at amino acid residues F116, L135, S174, S176, and/or T178 (EU numbering) and (b) the CH1₂ comprises one or more mutations at amino acid residues A141, F170, S181, S183, and/or V185 (EU numbering). In some aspects, the CL2 comprises one or more of the following substitution mutations: F116A, L135V, S174A, S176F, and/or T178V. In some aspects, the CL2 comprises the following substitution mutations: F116A, L135V, S174A, S176F, and T178V. In some aspects, the CH1₂ comprises one or more of the following substitution mutations: A141I, F170S, S181M, S183A, and/or V185A. In some aspects, the CH1₂ comprises the following substitution mutations: A141I, F170S, S181M, S183A, and V185A. In some aspects, the anti-FcRH5 antibody comprises one or more heavy chain constant domains, wherein the one or more heavy chain constant domains are selected from a first CH2 domain (CH2₁), a first CH3 domain (CH3₁), a second CH2 domain (CH2₂), and a second CH3 domain (CH3₂). In some aspects, at least one of the one or more heavy chain constant domains is paired with another heavy chain constant domain. In some aspects, the CH3₁ and the CH3₂ each comprise a protuberance (P₁) or a cavity (C₁), and wherein the P₁ or the C₁ in the CH3₁ is positionable in the C₁ or the P₁, respectively, in the CH3₂. In some aspects, the CH3₁ and the CH3₂ meet at an interface between the P₁ and the C₁. In some aspects, the CH2₁ and the CH2₂ each comprise (P₂) or a cavity (C₂), and wherein the P₂ or the C₂ in the CH2₁ is positionable in the C₂ or the P₂, respectively, in the CH2₂. In some aspects, the CH2₁ and the CH2₂ meet at an interface between the P₂ and the C₂.

### J. Recombinant Methods and Compositions

Antibodies disclosed herein, e.g., bispecific anti-FcRH5/anti-CD3 antibodies as disclosed herein, may be produced using recombinant methods and compositions, for example, as described in U.S. Pat. No. 4,816,567. In one embodiment, an isolated nucleic acid encoding an antibody, e.g., anti-FcRH5 antibody (e.g., a bispecific anti-FcRH5/anti-CD3 antibody) described herein is provided. Such nucleic acid may encode an amino acid sequence comprising the VL and/or an amino acid sequence comprising the VH of the antibody (e.g., the light and/or heavy chains of the antibody). In another embodiment, an isolated nucleic acid encoding an anti-CD3 antibody described herein is provided. Such a nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such a nucleic acid are provided. In a further embodiment, a host cell comprising such a nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g., a Chinese Hamster Ovary (CHO) cell or lymphoid cell (e.g., Y0, NS0, Sp20 cell). In one embodiment, a method of making an antibody, e.g., an bispecific anti-FcRH5/anti-CD3 antibody, is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody, e.g., a bispecific anti-FcRH5/anti-CD3 antibody, a nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

### 1. Two-Cell Methods for Manufacturing Bispecific Antibodies

In some aspects, an antibody as disclosed herein (e.g., a bispecific anti-FcRH5/anti-CD3 antibody) is manufactured using a method comprising two host cell lines. In some aspects, a first arm of the antibody (e.g., a first arm comprising a hole region) is produced in a first host cell line, and a second arm of the antibody (e.g., a second arm comprising a knob region) is produced in a second host cell line. The arms of the antibody are purified from the host cell lines and assembled in vitro.

### 2. One-Cell Methods for Manufacturing Bispecific Antibodies

In some aspects, an antibody as disclosed herein (e.g., a bispecific anti-FcRH5/anti-CD3 antibody) is manufactured using a method comprising a single host cell line. In some aspects, a first arm of the antibody (e.g., a first arm comprising a hole region) and a second arm of the antibody (e.g., a second arm comprising a knob region) are produced in and purified from a single host cell line. Preferably, the first arm and the second arm are expressed at comparable levels in the host cell, e.g., are both expressed at a high level in the host cell. Similar levels of expression increase the likelihood of efficient TDB production and decrease the likelihood of light chain (LC) mispairing of TDB components. The first arm and second arm of the antibody may each further comprise amino acid substitution mutations introducing charge pairs, as described in Section II(I)(7) herein. The charge pairs promote the pairing of heavy and light chain cognate pairs of each arm of the bispecific antibody, thereby minimizing mispairing.

### 3. Host Cells

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, NJ, 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, NJ), pp. 255-268 (2003).

### K. Immunoconjugates

The invention also provides immunoconjugates comprising an antibody as disclosed herein, e.g., a bispecific anti-FcRH5/anti-CD3 antibody disclosed herein, conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one embodiment, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another embodiment, an immunoconjugate comprises an antibody as disclosed herein, e.g., a bispecific anti-FcRH5/anti-CD3 antibody as described herein, conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica Charantia inhibitor, curcin, crotin, sapaonaria Officinalis inhibitor, gelonin, mitogellin, restrictocin, phenomycin, enomycin, and the tricothecenes.

In another embodiment, an immunoconjugate comprises an antibody as disclosed herein, e.g., a bispecific anti-FcRH5/anti-CD3 antibody described herein, conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, MRI), such as iodine-123 again, iodine-131, indium-111, fluorine-19, carbon-13, nitrogen-15, oxygen-17, gadolinium, manganese, or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A).

### L. Pharmaceutical Compositions and Formulations

Pharmaceutical compositions and formulations of the therapeutic agents described herein (e.g., anti-FcRH5/anti-CD3 bispecific antibodies and corticosteroids (e.g., dexamethasone or methylprednisolone)) can be prepared by mixing such therapeutic agents having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as L-Histidine/glacial acetic acid (e.g., at pH 5.8), phosphate, citrate, and other organic acids; tonicity agents, such as sucrose; stabilizers, such as L-methionine; antioxidants including N-acetyl-DL-tryptophan, ascorbic acid, and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polysorbate 20 or polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredient as necessary for the particular indication being treated, preferably those with complementary activities that do not adversely affect each other. For example, it may be desirable to further provide an additional therapeutic agent (e.g., a chemotherapeutic agent, a cytotoxic agent, a growth inhibitory agent, and/or an anti-hormonal agent, such as those recited herein above). Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles, and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, for example, films, or microcapsules.

The formulations to be used for in vivo administration are generally sterile. Sterility may be readily accomplished, e.g., by filtration through sterile filtration membranes.

### III. Articles of Manufacture

In another aspect of the invention, an article of manufacture containing materials useful for the treatment, prevention, and/or diagnosis of the disorders described above is provided. For example, an article of manufacture for use in any of the methods disclosed herein is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition may be an anti-FcRH5/anti-CD3 bispecific antibody described herein. In some examples, at least one active agent in the composition may be an anti-CD38 antibody (e.g., daratumumab), an IMiD (e.g., pomalidomide, a corticosteroid (e.g., dexamethasone or methylprednisolone), or a combination thereof.

In some aspects, the article of manufacture comprises at least two containers (e.g., vials), a first container holding an amount of the composition suitable for a C1D1 (cycle 1, dose 1) and a second container holding an amount of the composition suitable for a C1D2 (cycle 1, dose 2). In some aspects, the article of manufacture comprises at least three containers (e.g., vials), a first container holding an amount of the composition suitable for a C1D1, a second container holding an amount of the composition suitable for a C1D2, and a third container holding an amount of the composition suitable for a C1D3. In some aspects, the containers (e.g., vials) may be different sizes, e.g., may have sizes proportional to the amount of the composition they contain. Articles of manufacture comprising containers (e.g., vials) proportional to the intended doses may, e.g., increase convenience, minimize waste, and/or increase cost-effectiveness. The label or package insert indicates that the composition is used for treating the condition of choice (e.g., a multiple myeloma (MM), e.g., relapsed or refractory MM, e.g., 4L+ treatment for R/R MM) and further includes information related to at least one of the dosing regimens described herein. Moreover, the article of manufacture may comprise (a) a first container with a composition contained therein, wherein the composition comprises an anti-FcRH5/anti-CD3 bispecific antibody described herein; and (b) a second container with a composition contained therein, wherein the composition comprises a further cytotoxic or otherwise therapeutic agent. Alternatively, or additionally, the article of manufacture may further comprise a second (or third) container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In some aspects, the article of manufacture is for carrying out the methods of the invention, for example, a kit containing a bispecific antibody that binds to FcRH5 and CD3 for use in treating a subject having a R/R MM (e.g., a subject having a triple-class refractory MM). In some examples, a single target dose (e.g., 40 mg, 90 mg, 120 mg, 132 mg, 160 mg, 198 mg, or 252 mg) of the bispecific antibody is provided. In some examples, a first step-up dose (e.g., 3.3 mg or 3.6 mg) and a target dose (e.g., 40 mg, 90 mg, 120 mg, 132 mg, 160 mg, 198 mg, or 252 mg) of the bispecific antibody is provided. In some examples, a first step-up dose (e.g., 0.3 mg), a second step-up dose (e.g., 3.3 mg or 3.6 mg), and a target dose (e.g., 40 mg, 90 mg, 120 mg, 132 mg, 160 mg, 198 mg, or 252 mg) of the bispecific antibody is provided. In some examples, the bispecific antibody may be cevostamab. In some embodiments, the kit may further contain one or more reagents (e.g., a buffer, preservative, and/or diluent) suitable for storage and/or administration of the bispecific antibody. The bispecific antibody (e.g., cevostamab) and/or the one or more reagents may be in the form of a liquid or a lyophilized powder and stored within one or more containers. Kits may also include instructions for use.

For example, provided herein is a kit for treating a subject having an R/R MM, wherein the subject has a triple-class refractory MM and has previously received a BCMA-targeting therapeutic agent, the kit comprising a bispecific antibody that binds to FcRH5 and CD3, and instructions to administer the bispecific antibody to the subject in a dosing regimen comprising: (i) a first phase comprising administering the bispecific antibody to the subject in at least a first 21-day dosing cycle (C1), wherein the first phase comprises administration of the bispecific antibody to the subject on Day 1 and Day 2 of the C1; and (ii) a second phase comprising one or more 21-day dosing cycles, wherein the second phase comprises administering the bispecific antibody to the subject Q3W.

In another example, provided herein is a kit for treating a subject having an R/R MM, wherein the kit comprises a bispecific antibody that binds to FcRH5 and CD3 and instructions to administer the bispecific antibody to the subject in a dosing regimen comprising at least a first 21-day dosing cycle, wherein the first 21-day dosing cycle comprises a first dose (C1D1), a second dose (C1D2), and a third dose (C1D3) of the bispecific antibody, wherein the C1D1 is between about 0.2 mg to about 0.4 mg and is administered to the subject on Day 1 of the first dosing cycle, the C1D2 is about 3.1 mg to about 3.4 mg and is administered to the subject on Day 2 of the first dosing cycle, and the C1D3 is greater than the C1D2.

In another example, provided herein is a kit for treating a subject having an R/R MM, wherein the subject has a triple-class refractory MM and has previously received a BCMA-targeting TDB antibody, the kit comprising cevostamab and instructions to administer the cevostamab as a monotherapy to the subject in a dosing regimen comprising: (i) a first phase comprising administering the cevostamab to the subject in a first dosing cycle (C1); and (ii) a second phase comprising administering the cevostamab to the subject Q3W (e.g., until the subject experiences disease progression, unacceptable toxicity, or death), wherein each dosing cycle of the first and second phase is a 21-day dosing cycle, and the cevostamab is administered to the subject: (i) at a first step-up dose of 0.3 mg during the first phase on Day 1 of the C1 and as a second step-up dose of 3.3 mg during the first phase on Day 2 of the C1; (ii) at a target dose of 160 mg during the first phase on Day 8 of the C1; and (iii) at the target dose of 160 mg during the second phase on Day 1 of each dosing cycle.

In another example, provided herein is a kit for treating a subject having an R/R MM, wherein the subject has a triple-class refractory MM and has previously received a BCMA-targeting TDB antibody, the kit comprising cevostamab and instructions to administer the cevostamab as a monotherapy to the subject in a dosing regimen comprising: (i) a first phase comprising administering the cevostamab to the subject in a first dosing cycle (C1); and (ii) a second phase comprising administering the cevostamab to the subject Q3W (e.g., until the subject experiences disease progression, unacceptable toxicity, or death), wherein each dosing cycle of the first and second phase is a 21-day dosing cycle, and the cevostamab is administered to the subject: (i) at a first step-up dose of 0.3 mg during the first phase on Day 1 of the C1 and as a second step-up dose of 3.3 mg during the first phase on Day 2 of the C1; (ii) at a target dose of 160 mg during the first phase on Day 9 of the C1; and (iii) at the target dose of 160 mg during the second phase on Day 1 of each dosing cycle.

In another example, provided herein is a kit for treating a subject having an R/R MM, wherein the subject has a triple-class refractory MM and has previously received a BCMA-targeting TDB antibody, the kit comprising cevostamab and instructions to administer the cevostamab as a monotherapy to the subject in a dosing regimen comprising: (i) a first phase comprising administering the cevostamab to the subject in a first dosing cycle (C1); and (ii) a second phase comprising administering the cevostamab to the subject Q3W (e.g., until the subject experiences disease progression, unacceptable toxicity, or death), wherein each dosing cycle of the first and second phase is a 21-day dosing cycle, and cevostamab is administered to the subject: (i) at a first step-up dose of 0.3 mg during the first phase on Day 1 of the C1 and as a second step-up dose of 3.6 mg during the first phase on Day 2 of the C1; (ii) at a target dose of 160 mg during the first phase on Day 8 of the C1; and (iii) at the target dose of 160 mg during the second phase on Day 1 of each dosing cycle.

In another example, provided herein is a kit for treating a subject having an R/R MM, wherein the subject has a triple-class refractory MM and has previously received a BCMA-targeting TDB antibody, the kit comprising cevostamab and instructions to administer the cevostamab as a monotherapy to the subject in a dosing regimen comprising: (i) a first phase comprising administering the cevostamab to the subject in a first dosing cycle (C1); and (ii) a second phase comprising administering the cevostamab to the subject Q3W (e.g., until the subject experiences disease progression, unacceptable toxicity, or death), wherein each dosing cycle of the first and second phase is a 21-day dosing cycle, and cevostamab is administered to the subject: (i) at a first step-up dose of 0.3 mg during the first phase on Day 1 of the C1 and as a second step-up dose of 3.6 mg during the first phase on Day 2 of the C1; (ii) at a target dose of 160 mg during the first phase on Day 9 of the C1; and (iii) at the target dose of 160 mg during the second phase on Day 1 of each dosing cycle.

In another example, provided herein is a kit for treating a subject having an R/R MM, wherein the subject has a triple-class refractory MM and has previously received a BCMA-targeting CAR-T, the kit comprising cevostamab and instructions to administer the cevostamab as a monotherapy to the subject in a dosing regimen comprising: (i) a first phase comprising administering the cevostamab to the subject in a first dosing cycle (C1); and (ii) a second phase comprising administering the cevostamab to the subject Q3W (e.g., until the subject experiences disease progression, unacceptable toxicity, or death), wherein each dosing cycle of the first and second phase is a 21-day dosing cycle, and the cevostamab is administered to the subject: (i) at a first step-up dose of 0.3 mg during the first phase on Day 1 of the C1 and as a second step-up dose of 3.3 mg during the first phase on Day 2 of the C1; (ii) at a target dose of 160 mg during the first phase on Day 8 of the C1; and (iii) at the target dose of 160 mg during the second phase on Day 1 of each dosing cycle.

In another example, provided herein is a kit for treating a subject having an R/R MM, wherein the subject has a triple-class refractory MM and has previously received a BCMA-targeting CAR-T, the kit comprising cevostamab and instructions to administer the cevostamab as a monotherapy to the subject in a dosing regimen comprising: (i) a first phase comprising administering the cevostamab to the subject in a first dosing cycle (C1); and (ii) a second phase comprising administering the cevostamab to the subject Q3W (e.g., until the subject experiences disease progression, unacceptable toxicity, or death), wherein each dosing cycle of the first and second phase is a 21-day dosing cycle, and the cevostamab is administered to the subject: (i) at a first step-up dose of 0.3 mg during the first phase on Day 1 of the C1 and as a second step-up dose of 3.3 mg during the first phase on Day 2 of the C1; (ii) at a target dose of 160 mg during the first phase on Day 9 of the C1; and (iii) at the target dose of 160 mg during the second phase on Day 1 of each dosing cycle.

In another example, provided herein is a kit for treating a subject having an R/R MM, wherein the subject has a triple-class refractory MM and has previously received a BCMA-targeting ADC, the kit comprising cevostamab and instructions to administer the cevostamab as a monotherapy to the subject in a dosing regimen comprising: (i) a first phase comprising administering the cevostamab to the subject in a first dosing cycle (C1); and (ii) a second phase comprising administering the cevostamab to the subject Q3W (e.g., until the subject experiences disease progression, unacceptable toxicity, or death), wherein each dosing cycle of the first and second phase is a 21-day dosing cycle, and the cevostamab is administered to the subject: (i) at a first step-up dose of 0.3 mg during the first phase on Day 1 of the C1 and as a second step-up dose of 3.3 mg during the first phase on Day 2 of the C1; (ii) at a target dose of 160 mg during the first phase on Day 8 of the C1; and (iii) at the target dose of 160 mg during the second phase on Day 1 of each dosing cycle.

In another example, provided herein is a kit for treating a subject having an R/R MM, wherein the subject has a triple-class refractory MM and has previously received a BCMA-targeting ADC, the kit comprising cevostamab and instructions to administer the cevostamab as a monotherapy to the subject in a dosing regimen comprising: (i) a first phase comprising administering the cevostamab to the subject in a first dosing cycle (C1); and (ii) a second phase comprising administering the cevostamab to the subject Q3W (e.g., until the subject experiences disease progression, unacceptable toxicity, or death), wherein each dosing cycle of the first and second phase is a 21-day dosing cycle, and the cevostamab is administered to the subject: (i) at a first step-up dose of 0.3 mg during the first phase on Day 1 of the C1 and as a second step-up dose of 3.3 mg during the first phase on Day 2 of the C1; (ii) at a target dose of 160 mg during the first phase on Day 9 of the C1; and (iii) at the target dose of 160 mg during the second phase on Day 1 of each dosing cycle.

In another example, provided herein is a kit containing a bispecific antibody that binds to FcRH5 and CD3 for use in treatment of a subject having a R/R MM, wherein the subject has a triple class refractory MM and has previously received a BCMA-targeting therapeutic agent, the treatment comprising administration of the bispecific antibody to the subject in a dosing regimen comprising: (i) a first phase comprising a first 21-day dosing cycle (C1), wherein the first phase comprises administering the bispecific antibody to the subject on Day 1, Day 2, and Day 8 of the C1; and (ii) a second phase comprising one or more 21-day dosing cycles, wherein the second phase comprises administering the bispecific antibody to the subject Q3W.

In another example, provided herein is a kit containing a bispecific antibody that binds to FcRH5 and CD3 for use in treatment of a subject having a R/R MM, wherein the subject has a triple class refractory MM and has previously received a BCMA-targeting therapeutic agent, the treatment comprising administration of the bispecific antibody to the subject in a dosing regimen comprising: (i) a first phase comprising a first 21-day dosing cycle (C1), wherein the first phase comprises administering the bispecific antibody to the subject on Day 1, Day 2, and Day 9 of the C1; and (ii) a second phase comprising one or more 21-day dosing cycles, wherein the second phase comprises administering the bispecific antibody to the subject Q3W.

In another example, provided herein is a kit containing a bispecific antibody that binds to FcRH5 and CD3 for use in treatment of a subject having an R/R MM, the treatment comprising administering to the subject a bispecific antibody that binds to FcRH5 and CD3 in a dosing regimen comprising at least a first 21-day dosing cycle, wherein the first dosing cycle comprises a first dose (C1D1), a second dose (C1D2), and a third dose (C1D3) of the bispecific antibody, wherein the C1D1 is between about 0.2 mg to about 0.4 mg and is administered to the subject on Day 1 of the first dosing cycle, the C1D2 is about 3.1 mg to about 3.4 mg and is administered to the subject on Day 2 of the first dosing cycle, and the C1D3 is greater than the C1D2.

### IV. Example

The following is an example of the methods of the invention. It is understood that various other embodiments may be practiced, given the general description provided above, and the example is not intended to limit the scope of the claims.

### Example 1. A Phase I/II, Open-Label, Multicohort Study to Evaluate the Efficacy and Safety of Cevostamab in Prior B Cell Maturation Antigen (BCMA)-Exposed Patients with Relapsed or Refractory (R/R) Multiple Myeloma (MM)

This example describes protocol number CO43476 (referred herein as “CAMMA 2”), a multicenter, multi-cohort, non-randomized, open-label, Phase I/II trial investigating the efficacy, safety, pharmacokinetics, pharmacodynamics, and immunogenicity of cevostamab in patients with triple class refractory multiple myeloma (MM) and prior exposure to a B cell maturation factor (BCMA)-targeted agent. Approximately 120-140 participants will be enrolled in this study.

Multiple myeloma remains an incurable malignancy, and most patients eventually become refractory to currently available treatments. Combination regimens with two or more drugs, including proteasome inhibitors (Pis), immunomodulatory drugs (IMiDs), and/or monoclonal antibodies (mAbs) targeting cell surface proteins such as cluster of differentiation 38 (CD38), are commonly used in all lines of treatment for MM but have decreasing response rates and shorter durations of response (DOR) after re-exposure to previously received classes of therapy. The most recent salvage therapies approved by the U.S. Food and Drug Administration (FDA) and European Medicines Agency (EMA) are B cell maturation antigen (BCMA)-targeted agents: BLENREP® (belantamab mafodotin), an antibody-drug conjugate (ADC), and ABECMA® (idecabtagene-vicleucel), an autologous chimeric antigen receptor T (CAR-T). Beyond these therapies, patients are left with limited options. Therefore, there is an unmet need for novel treatments for patients with multiple relapses or refractory (R/R) disease after exposure to a PI, IMiD, anti-CD38 mAb, and a BCMA-targeted agent.

### A. Objectives and Endpoints

This study will evaluate the efficacy, safety, and pharmacokinetics of cevostamab in participants with R/R MM. Sections A(i) and A(ii) below present the primary and secondary efficacy objectives for the study expressed using the estimand framework in accordance with the International Conference for Harmonization (ICH) E9 (R1) statistical principles for clinical trials (FDA 2021 b). Specific objectives and corresponding endpoints for the study are outlined in Table 5.

i. Primary Efficacy Objective
Population: participants with R/R MM who are refractory to at least a PI, an IMiD, and an anti-CD38 agent with prior exposure to a BCMA-targeted ADC or CAR-T (Cohorts A1 and B1) or a Bispecific antibody (Cohorts A2 and B2), as defined through the inclusion and exclusion criteria, who received at least 1 dose of study treatment. The primary efficacy analysis will be performed on participants with prior exposure to ADC or CAR-T treatment from Cohort B1.
Variable: Objective response rate (ORR), i.e., the proportion of participants achieving stringent complete response (sCR), complete response (CR), very good partial response (VGPR) or partial response (PR) as assessed by the investigator according to the 2016 International Myeloma Working Group (IMWG) criteria (see Table 6A and Table 6B).
Summary measure (estimate): the estimated ORR and its 95% confidence interval (CI) using the Clopper-Pearson methodology.
   i. Secondary Efficacy Objectives

The estimand components for the secondary efficacy endpoints ORR assessed by Independent Review Committee (IRC) and tumor responses CR or better, and VGPR or better by investigator and by IRC are defined as above for the primary efficacy estimand. The estimand components for the secondary efficacy endpoint of DOR is defined as follows:
Population: participants as defined for the primary analysis estimand who achieved an objective response (sCR, CR, VGPR or PR)
Variable: the time interval from the date of the first occurrence of an objective response until the first date that progressive disease (PD) or death from any cause is documented, whichever occurs first.
Summary measure: the survival event rates associated with DOR at particular landmarks (i.e., every 3 months and median if reached).
The estimand components for the secondary efficacy endpoints of progression-free survival (PFS), OS, time to best response (TBR) and time to first response (TTR) are defined as follows:
Population: participants from the primary estimand.
Variable: time from first administration of the study treatment to the first occurrence of the respective event of interest.
Summary measure: survival event rates associated with the respective endpoints at particular landmarks (i.e., every 3 months and median if reached).

The estimand components for the secondary efficacy endpoint minimal residual disease (MRD) negativity is defined as follows:
Population: participants from the primary estimand who achieve a CR or sCR per investigator.
T Variable: proportion of participants who are MRD negative (<10⁻⁵) by next-generation sequencing (NGS).
Summary measure: the estimated proportion of participants who are MRD-negative and its 95% Cl using the Clopper Pearson methodology.

Specific objectives and corresponding endpoints for the study are outlined in Table 5.

TABLE 5
Objectives and Endpoints

| | Corresponding Endpoints |
|---|---|
| Primary Objectives | |
| To evaluate the efficacy of cevostamab. | ORR, defined as the proportion of participants |
| | with an objective response (sCR, CR, VGPR, or |
| | PR), based on investigator-assessed response, |
| | according to IMWG criteria (Kumar et al., Lancet |
| | Oncol,, 17: e328-46, 2016). |
| To evaluate the safety and tolerability of | Incidence and severity of adverse events, with |
| cevostamab. | severity determined according to NCI CTCAE |
| | v5.0 and American Society of Transplantation |
| | and cellular Therapy (ASTCT) for cytokine |
| | release syndrome (CRS) and of immune effector |
| | cell-associated neurotoxicity syndrome (ICANS) |
| | grading (Lee et al., Biol, Blood, 25(4): 625-638, |
| | 2019). |
| Secondary Objective | |
| To evaluate the efficacy of cevostamab. | Note: IMWG Criteria will be used for the following |
| | endpoints: |
| | ORR, defined as the proportion of participants |
| | with an objective response based on IRC- |
| | assessed response. |
| | DOR, defined as the time from the first |
| | occurrence of an objective response until the |
| | date of disease progression or death from any |
| | cause (whichever occurs first), as determined |
| | separately by the IRC and the investigator. |
| | Rate of CR or better, defined as the proportion of |
| | participants who achieve a response of sCR or |
| | CR, as assessed separately by IRC and the |
| | investigator. |
| | Rate of VGPR or better, defined as the |
| | proportion of participants who achieve a |
| | response of VGPR or better, as assessed |
| | separately by IRC and the investigator. |
| | OS, defined as the time from initiation of study |
| | treatment to death from any cause. |
| To evaluate the efficacy of cevostamab. | PFS, defined as the time from initiation of study |
| | treatment to the first occurrence of disease |
| | progression, relapse, or death from any cause |
| | (whichever occurs first), as assessed separately |
| | by IRC and the investigator. |
| | Time to first response (for participants who |
| | achieve an objective response), defined as time |
| | from initiation of study treatment to first achieving |
| | an objective response (separate analyses by IRC |
| | and investigator). |
| | Time to best response (for participants who |
| | achieve an objective response), defined as time |
| | from initiation of study treatment to achieving the |
| | deepest response (separate analyses by IRC |
| | and investigator). |
| | MRD-negative rate, defined as proportion of |
| | participants achieving MRD negativity (<10⁻⁵), as |
| | defined by NGS in bone marrow aspirate per |
| | IMWG criteria (Kumar et al., Lancet Oncol, |
| | 17: e328-46, 2016) among participants with a |
| | response of CR or better (separate analyses for |
| | CR by IRC and investigator). MRD at VGPR if |
| | CR not achieved within 3 months of ongoing |
| | VGPR response. Analysis for this subpopulation |
| | will be exploratory. |
| | Proportion of participants experiencing a clinically |
| | meaningful improvement in the fatigue domain of |
| | the European Organisation for Research and |
| | Treatment of Cancer (EORTC) Quality of Life |
| | Core-30 Questionnaire (QLQ-C30) and/or |
| | disease symptoms domain of the EORTC QLQ- |
| | MY20. |
| | Time to deterioration in the fatigue domain of the |
| | EORTC QLQ-C30 and/or disease symptoms |
| | domain of the EORTC QLQ-MY20. |
| Pharmacokinetic Objectives | |
| To characterize the pharmacokinetic (PK) | Serum concentration of cevostamab at specified |
| of cevostamab in participants with MM. | timepoints. |
| | PK parameters of cevostamab, as data allow. |
| To evaluate the immune response to | Prevalence of anti-drug antibodies (ADAs) |
| cevostamab. | against cevostamab at baseline and incidence of |
| | ADAs against cevostamab during the study. |
| Exploratory Objectives | |
| To evaluate potential relationships between | Relationship between selected covariates and |
| selected covariates and exposure to | serum concentration or PK parameters for |
| cevostamab. | cevostamab. |
| To evaluate potential effects of ADAs | Relationship between ADA status and efficacy, |
| against cevostamab. | safety, or PK endpoints. |
| To evaluate the health-related quality of life | Proportion of participants experiencing a clinical |
| of participants treated with cevostamab | change from baseline in fatigue as measured by |
| (expansion cohorts only). | the EORTC QLQ-C30. |
| | Change from baseline in disease symptoms as |
| | measured by the EORTC QLQ-MY20. |
| To evaluate the health status of | Change from baseline in EQ-5D-5L index-based |
| participants treated with cevostamab | and Visual Analog Scale (VAS) scores. |
| (expansion cohorts only). | |

### B. Study Design

This is a prospective, multicenter, multi-cohort, non-randomized, open-label, Phase I/II trial investigating the efficacy and safety of cevostamab in patients with triple class refractory MM and prior exposure to a BCMA-targeted agent (see Table 5 for objectives and endpoints). Approximately 120-140 participants will be enrolled in this study.

Potential participants with R/R MM who meet the eligibility criteria will be enrolled in one of two parallel cohorts, as defined below:
1. Prior BCMA ADC or CAR-T Cohort: will enroll participants who have previously received a BCMA-targeting ADC or BCMA-targeting CAR-T-cell therapy and are triple-class refractory (at least a PI, an IMiD, and an anti-CD38 mAb). An initial exploratory Cohort (A1) comprised of 10-20 participants will be treated at the double step-up split dosing regimen of 0.3 mg (step-up split dose 1), 3.3 mg (step-up split dose 2), and 160 mg (target dose; TD), referred herein as “0.3/3.3/160 mg.” An expansion Cohort (B1) will enroll approximately 80 participants at the recommended Phase 2 dose (RP2D) for monotherapy identified on collective data from ongoing Phase I dose escalation studies, including the dose escalation Study GO39775 (ClinicalTrials.gov Identifier: NCT03275103). The split of participants in Cohorts A1 and B1 will be approximately 1:1 of participants previously treated with an ADC and participants previously treated with a CAR-T.
2. Prior BCMA Bispecific Cohort: will enroll participants who have previously received a BCMA-targeting T cell dependent bispecific (TDB) antibody therapy and are triple-class refractory (at least a PI, an IMiD, and an anti-CD38 mAb). An initial exploratory Cohort A2 (dosing regimen of 0.3/3.3/160 mg) will enroll approximately 10-20 participants. Based on the results of the initial 10-20 participants, an expansion Cohort B2 at the same dose as per Cohort B1 (RP2D) will be opened.

Participants with prior exposure to a BCMA-targeting TDB antibody will only be enrolled in the Prior BCMA Bispecific cohort. Participants with prior exposure to ADC and CAR-T and a bispecific will also be enrolled in the Prior BCMA Bispecific Cohort.

Participants in Cohorts A1 and B1 will receive cevostamab as a single agent by IV infusion in 21-day cycles. To mitigate the risk of CRS, cevostamab will be administered as a step-up split dosing regimen of 0.3 mg on Day 1 and 3.3 mg on Day 2 of Cycle 1 if no CRS event occurs after the initial dose on Day 1. A minimum of 20 hours from the end of infusion of the previous cevostamab dose to initiation of the next dose will be respected for doses that are administered 1 day apart. If the participant experiences CRS following the step-up dose of 0.3 mg, the next dose (3.3 mg) will be delivered on Day 2, 3, or 4, upon full resolution of CRS. Additional dose delay may be necessary depending on the clinical presentation of the CRS event. The TD of 160 mg is administered on Day 8 of Cycle 1. For the TD, additional dose delay may be necessary depending on the clinical presentation of the CRS event. For example, the TD of 160 mg may be administered on or after Day 9 of Cycle 1. The Cycle 2, Day 1 (C2D1) dose must be given a minimum of 7 days after the TD is given in Cycle 1. Target doses can then be administered on Day 1 of subsequent cycles until disease progression, unacceptable toxicity, withdrawal of consent, or death, whichever occurs first. All enrolled participants will be required to be hospitalized after the completion of each cevostamab infusion during Cycle 1 (step-up split dose 1 and 2, and first TD for at least 48 hours after completion of the infusion, and until there is no evidence of ongoing CRS or neurological toxicity, vital signs and oxygen saturation levels have returned to baseline, and all abnormal laboratory values and adverse events have resolved. Once the participant has completed a cycle of cevostamab at the TD infusion without an IRR or CRS, subsequent infusions may be delivered without hospitalization. ACTEMRA®/ROACTEMRA® (tocilizumab) will be administered per protocol guidance when necessary to participants who experience treatment emergent CRS.

Cohorts B1 and B2 will be open at the RP2D for monotherapy based on data from collective Phase I studies performed across the program as well as the preliminary efficacy observed in split step-up dose Cohorts A1 and A2. Each cohort can be opened independently.

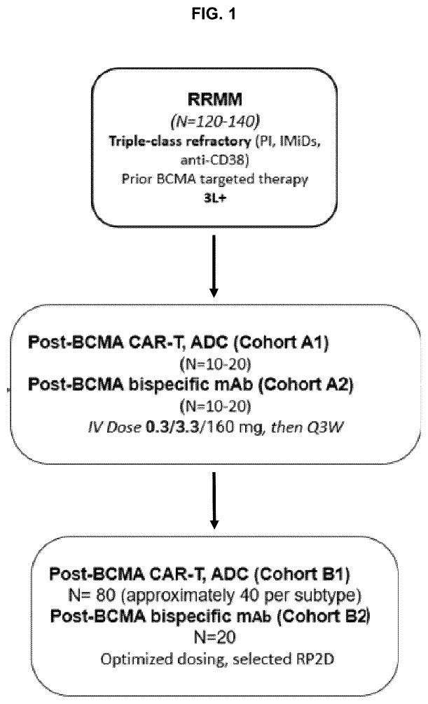

The first 3 participants in Cohorts A will be recruited in a staggered way, with only 1 participant at a time during the Cycle 1 step-up dosing timeframe Days 1-5. The next 3 participants will not be recruited simultaneously but again, staggered with at least a 24-hour interval. A study schema is provided in FIG. 1.

Disease assessments on Day 1 of each cycle will be performed according to the IMWG response criteria (e.g., Table 6A and Table 6B) by the investigator and an IRC. The IRC will perform a blinded independent central review of serum protein electrophoresis (SPEP), serum free light chain assays (sFLC), urinary protein electrophoresis (UPEP), bone marrow biopsies/aspirate, images, and other clinical data as needed.

The following stopping criteria for safety will apply across all cohorts of the study:
Any Grade 5 adverse event that is not attributable to another clearly identifiable cause.
Any Grade ≥4 CRS event
Any Grade ≥4 neurotoxicity and Grade ≥3 seizure that are not considered by the investigator to be attributable to another clearly identifiable cause.
One patient in exploratory Cohort A or 10% of patients overall (of a minimum of 12 patients enrolled in the study) experiences any Grade ≥4 confirmed HLH/MAS.
   ii. Rationale for Study Design

Despite advances in therapy, MM remains an incurable malignancy, and most patients eventually become refractory to currently available treatments. There is no single standard of care for R/R MM, and treatment is affected by several factors, including age, performance status, comorbidities, and the type, efficacy, and tolerance of the previous treatments. In general, it is recommended to avoid re-treatment with the same agent and/or class of agent in the subsequent line of treatment (Laubach et al., Leukemia, 30:1005-17, 2016; Moreau et al., Lancet Oncol, 22:e105-18, 2021). With Pis, IMiDs, and anti-CD38 mAbs included in early lines of treatment as a standard of care, therapeutic options for patients in second or subsequent relapses become more limited and challenging (Dimopoulos et al., Leukemia, 35; 1722-31, 2021).

BCMA-targeting therapies have recently been adopted as a new option in the treatment of patients with R/R MM. Belantamab mafodotin, an ADC, is the first BCMA-targeted therapy that was granted accelerated approval in August 2020 by the FDA for the treatment of adults with R/R MM who have received at least 4 prior therapies, including a PI, IMiD, and an anti-CD38 mAb. Conditional approval by the EMA, also granted in August 2020, is for the treatment of adult patients with MM who have received at least 4 previous treatment lines and whose disease is refractory to treatment with at least a PI, an IMiD, and an anti-CD38 mAb, and who have had disease progression on their last therapy. Patients treated with the approved belantamab mafodotin dose of 2.5 mg/kg IV every 3 weeks in the pivotal DREAMM-2 study showed an ORR of 31%, a median DOR of 11 months (range: 4.2 to not evaluable [NE]), and a median PFS of 2.8 months. An updated analysis reported an OS of 13.7 months (Lonial et al., Lancet Oncol, 21:207-21, 2020; Lonial et al., Cancer, 127:4198-212, 2021). Idecabtagene vicleucel, a BCMA-targeted CAR T-cell therapy, was approved by the FDA in March 2021, based on the KarMMa trial, for the treatment of adults with R/R MM after 4 or more prior therapies, including a PI, an IMiD, and an anti-CD38 mAb. Patients treated with ide-cel showed an ORR of 73%, a median PFS of 8.8 months, and a median OS of 19.4 months with a median follow-up of 13.3 months (Munshi et al., N Engl J Med, 384:705-16, 2021). Other BCMA-targeted therapies are currently in development, in particular CAR T-cell therapies, including ciltacabtagene autoleucel (cilta-cel), and other bispecific antibody therapies. Hence, the proportion of patients receiving these therapies are expected to increase in the coming years and the post-BCMA participant segment will become a new unmet medical need.

There are currently no approved therapies for patients who relapse following BCMA-targeted therapy, and data on patients who progress or relapse after BCMA-targeting agents are limited. While resistance mechanisms to BCMA-targeted therapies are not fully understood, emerging data suggest loss of BCMA as one mechanism, with subclonal BCMA gene deletion and dominant outgrowth of preexisting BCMA-negative or low-expressing subclones after selective pressure generated by anti-BCMA therapies as possible other causes (Samur et al., Nat. Commun, 12:868, 2021; Truger et al., Blood Adv., 5:3794-8, 2021). A retrospective single-center chart review analyzed the outcomes of 47 patients who received BCMA-targeted therapy: with a median follow-up time of 6 months, 22 patients (46.8%) had disease progression and 18 patients received subsequent therapy. Among the 18 patients who received subsequent therapy following BCMA-targeted treatment (e.g., with infusion chemotherapy, elotuzumab- and selinexor-based regimens), the estimated 12-month OS rate was 51.5% (Paulet al. Efficacy of subsequent therapies in multiple myeloma patients after progression on a BCMA targeting therapy: a single-center experience. 2020. Available from: ash.confex.com/ash/2020/webprogram/Paper141637.html.). Updated reports with a focus on 28 patients post-CAR-T treatment, showed the best response to initial treatment at 46% ORR (7 CR, 5 VGPR, 1 PR, 7 stable disease (SD), 8 PD). Median time to progression was 105 days (95% CI: 78 to 204) for the initial treatment after CAR-T (Van Oekelen et al., Blood, 138 (Suppl 1):2704, 2021). The majority of these patients received ≥2 lines of subsequent therapies, suggesting that durable responses are difficult to attain with regimens available at this stage of treatment, and due to the absence of a standard of care, the choice of therapy may be dependent on multiple factors, including toxicities, patients' comorbidities, and institutional/physician preference. Early data suggest that CAR-T re-treatment is associated with limited benefit. Of 28 patients who were retreated with ide-cel in the KarMMa study, only 6 patients (21%) achieved a second response, with DOR ranging from 1.9-6.8 months (Munshi et al., N. Engl. J. Med., 384:705-16, 2021).

Therefore, the development of more effective therapeutic interventions with novel mechanisms of action and new target for patients who have failed to respond to or relapsed after treatment with prior BCMA-targeted therapy is a key priority.

This study (CO43476) will enroll participants who have previously received BCMA-targeted therapy into two cohorts based on the class of drug, a Prior BCMA ADC or CAR-T cohort and a Prior BCMA Bispecific cohort, as detailed in FIG. 1. Participants who received a BCMA-targeted TDB antibody are included in a separate exploratory Prior BCMA Bispecific cohort because BCMA-targeted T-cell bispecifics are still in early development and little is known about TDB antibody sequencing.

iii. Rationale for Cevostamab Treatment in Participants with Relapsed or Refractory Multiple Myeloma after Prior BCMA-Targeted Therapy

Cevostamab is a humanized, full-length IgG1 TDB antibody that binds FcRH5 with 1 arm and binds CD3 on the surface of T cells with the other arm. Nonclinical studies have found FcRH5 to be selectively expressed by B cells, plasma cells and MM cells, with no known expression in other tissues. Cevostamab is active against FcRH5-expressing cells, and low levels of FcRH5 expression on target cells is sufficient for cell killing. The clinical activity of cevostamab in R/R MM has been demonstrated in the ongoing Phase I, multicenter, open-label, dose-escalation study (Study GO39775) evaluating the safety and pharmacokinetics of cevostamab in patients with R/R MM for whom no established therapy for MM is appropriate and available, or who are intolerant to those established therapies.

In summary, patients with R/R MM, particularly those who are triple-class refractory and have received a BCMA-targeting agent, have few treatment options and represent an area of high unmet need. Cevostamab may provide a meaningful benefit over existing therapies and the favorable benefit-risk profile observed with cevostamab treatment supports further evaluation of cevostamab in these selected populations of R/R MM.

iv. Rationale for Primary Endpoint

The primary efficacy endpoint is investigator-assessed ORR, defined as the proportion of participants with an objective response (sCR, CR, VGPR, or PR) based on the IMWG criteria (e.g., Table 6A and Table 6B).

TABLE 6A
International Myeloma Working Group Uniform Response Criteria (2016)
Adapted from Durie et al. Leukemia 2015; 29: 2416-7 and Kumar et al. Lancet Oncol. 2016; 17: e328-46.

| | |
|---|---|
| Response | |
| Subcategory | Response Criteria |

All response categories require two consecutive assessments
made any time before starting any new therapy.

| | |
|---|---|
| sCR | CR as defined below, plus: |
| | Normal FLC ratio and absence of clonal cells in BM by immunohistochemistry |
| | (kappa/lambda ratio ≤4:1 or ≥1:2 for kappa and lambda patients, respectively after |
| | counting ≥100 plasma cells) ^{a}. |
| CR | No evidence of initial monoclonal protein isotype(s) on immunofixation of the serum |
| | and urine, ^{b} disappearance of any soft tissue plasmacytomas, and ≤5% plasma cells |
| | in BM |
| VGPR | Serum and urine M-protein detectable by immunofixation but not on electrophoresis; |
| | or ≥90% reduction in serum M-protein plus urine M-protein level <100 mg/24 hr ^{c} |
| PR | ≥50% reduction of serum M-protein and reduction in 24-hour urine M-protein |
| | by ≥90% or to <200 mg/24 hr |
| | If the serum and urine M-protein are unmeasurable, a ≥50% decrease in the |
| | difference between involved and uninvolved FLC levels is required in place of the M- |
| | protein criteria. |
| | If serum and urine M-protein are unmeasurable and serum FLC assay is also |
| | unmeasurable, ≥50% reduction in plasma cells is required in place of M-protein, |
| | provided baseline BM plasma cell percentage was ≥30%. |
| | In addition to the above listed criteria, if present at baseline, a ≥50% reduction in the |
| | size (SPD) ^{d} of soft tissue plasmacytomas is also required. |
| MR | ≥25% but ≤49% reductions of serum M-protein and reduction in 24-hour urine M-protein by |
| | 50%-89% |
| | In addition to the above criteria, if present at baseline, 25%-49% reduction in the |
| | size (SPD) ^{d} of soft tissue plasmacytomas is also required. |
| SD | Not meeting criteria for MR, CR, VGPR, PR, or PD. |

TABLE 6B
International Myeloma Working Group Uniform Response Criteria (2016)
Adapted from Durie et al. Leukemia 2015; 29: 2416-7 and Kumar et al. Lancet Oncol. 2016; 17: e328-46

| Relapse | |
|---|---|
| Subcategory | Relapse Criteria |
| PD ^{e, f} | Any one or more of the following criteria: |
| | Increase of ≥25% from lowest response value in one or more of the following: |
| | Serum M-protein (absolute increase must be ≥0.5 g/dL). |
| | Serum M-protein increase ≥1 g/dL, if the lowest M component was ≥5 g/dL. |
| | Urine M-protein (absolute increase must be ≥200 mg/24 hr). |
| | In patients without measurable serum and urine M-protein levels: the |
| | difference between involved and uninvolved FLC levels (absolute increase |
| | must be >10 mg/dL). |
| | In patients without measurable serum and urine M-protein levels and without |
| | measurable disease by FLC: BM plasma cell percentage irrespective of |
| | baseline status (absolute % must be ≥10%) ^{b}. |
| | Appearance of new lesion(s), ≥50% increase from nadir in SPD of >1 lesion, |
| | or ≥50% increase in the longest diameter of a previous lesion >1 cm in short |
| | axis. |
| | ≥50% increase in circulating plasma cells (minimum 200 cells per microliter) if this |
| | is the only measure of disease. |
| Clinical relapse | Requires one or more of the following: |
| | Direct indications of increasing disease and/or end organ dysfunction (CRAB |
| | features) ^{g} related to the underlying clonal plasma cell proliferative disorder. It |
| | is not used in calculation of time to progression or PFS but is listed here as |
| | something that can be reported optionally or for use in clinical practice. |
| | Development of new soft tissue plasmacytomas or bone lesions (osteoporotic |
| | fractures do not constitute progression). |
| | Definite increase in the size of existing plasmacytomas or bone lesions. A definite |
| | increase is defined as a 50% (and ≥1 cm) increase as measured serially by the |
| | sum of the products of the cross-diameters of the measurable lesion. |
| | Hypercalcemia >11 mg/dL (2.65 mmol/L). |
| | Decrease in hemoglobin of ≥2 g/dL (1.25 mmol/L) not related to therapy or other |
| | non-myeloma related conditions. |
| | Rise in serum creatinine by 2 mg/dL or more (177 μmol/L or more) from the start |
| | of therapy and attributable to myeloma. |
| | Hyperviscosity related to serum paraprotein. |
| Relapse from CR (to | Any one or more of the following: |
| be used only if the | Reappearance of serum or urine M-protein by immunofixation or electrophoresis |
| endpoint studied is | Development of ≥5% plasma cells in the BM. |
| PFS)^{d} | Appearance of any other sign of progression (i.e., new plasmacytoma, lytic |
| | bone lesion, or hypercalcemia). |

BM = bone marrow; CR = complete response; CT = computed tomography; FLC = free light chain; M-protein = monoclonal protein; MR = minimal response; MRI = magnetic resonance imaging; PD = progressive disease; PET = positron emission tomography; PFS = progression-free survival; PR = partial response; sCR = stringent complete response; SD = stable disease; SPD = sum of the products of diameters; VGPR = very good partial response.
Note:
Patients should be categorized as having stable disease until they meet criteria for any response category or have progressive disease. Patients will continue in the last confirmed response category until there is confirmation of progression or improvement to a higher response status; patients cannot move to a lower response category.
^{a} Special attention should be given to the emergence of a different M-protein following treatment, especially in the setting of patients having achieved a conventional CR, often related to oligoclonal reconstitution of the immune system. These bands typically disappear over time, and in some studies, have been associated with a better outcome. Also, appearance of IgGk in patients receiving monoclonal antibodies should be differentiated from the therapeutic antibody.
^{b} In some cases it is possible that the original M-protein light-chain isotype is still detected on immunofixation, but the accompanying heavy-chain component has disappeared; this would not be considered a CR even though the heavy-chain component is not detectable, since it is possible that the clone evolved to one that secreted only light chains. Thus, if a patient has IgA lambda myeloma, then to qualify as a CR there should beno IgA detectable on serum or urine immunofixation; if free lambda is detected without IgA, then it must be accompanied by a different heavy-chain isotype (IgG, IgM, etc.). Modified from Durie et al. Leukemia; 20: 1467-73 2006. Requires two consecutive assessments to be carried out at any time before the institution of any new therapy (Durie et al. Leukemia 2015; 29: 2416-7).
^{c} For patients achieving very good partial response by other criteria, a soft tissue plasmacytoma must decrease by more than 90% in the sum of the maximal perpendicular diameter (SPD) compared with baseline.
^{d}Plasmacytoma measurements should be taken from the CT portion of the PET/CT or MRI scans, or dedicated CT scans where applicable. For patients with only skin involvement, the skin lesions should be measured with a ruler. Measurement of tumor size will be determined by the SPD. Any soft tissue plasmacytoma documented at baseline must undergo serial monitoring; otherwise, the patient is classified as not evaluable.
^{e} Positive immunofixation alone in a patient previously classified as achieving a CR will not be considered progression. Criteria for relapse from aCR should be used only when calculating disease-free survival.
^{f} In the case where a value is felt to be a spurious result per investigator discretion (e.g., a possible laboratory error), that value will not beconsidered when determining the lowest value.
^{g} CRAB features = calcium elevation, renal failure, anemia, lytic bone lesions.

Objective response rate is recognized as an acceptable primary endpoint for early phase clinical trials and single-arm studies.

v. Rationale for Treatment of Cytokine Release Syndrome Using Tocilizumab

Cytokine release syndrome is a potentially life-threatening symptom complex, caused by the excessive release of cytokines by immune effector or target cells during an exaggerated and sustained immune response. Cytokine release syndrome can be triggered by a variety of factors, including infection with virulent pathogens, or by medications that activate or enhance the immune response, resulting in a pronounced and sustained immune response.

Regardless of the inciting agent, severe or life-threatening CRS is a medical emergency. If unsuccessfully managed, it can result in significant disability or is fatal.

Cytokine release syndrome is associated with elevations in a wide array of cytokines, including marked elevations in interferon (IFN)-γ, interleukin (IL)-6, and tumor necrosis factor (TNF)-α levels. Emerging evidence implicates IL-6 as a central mediator in CRS. Interleukin 6 is a proinflammatory multi-functional cytokine produced by a variety of cell types, which has been shown to be involved in a diverse array of physiological processes, including T-cell activation. Regardless of the inciting agent, CRS is associated with high IL-6 levels (Panelli et al., J Transl Med., 2:17, 2004; Lee et al., Blood, 124: 188-195, 2014; Doessegger and Banholzer, Clin Transl Immunology, 4:e39, 2015), and IL-6 correlates with the severity of CRS. Participants who experience severe or life-threatening CRS (NCI CTCAE Grades 4 or 5) have much higher IL-6 levels compared with those who experience milder or no CRS reactions (NCI CTCAE Grades 0-3; Chen et al., J Immunol Methods, 434:1-8, 2016).

Tocilizumab is a recombinant, humanized, anti-human mAb directed against soluble and membrane-bound IL-6 receptor (IL-6R), which inhibits IL-6-mediated signaling. Blocking the inflammatory action of IL-6 using tocilizumab can be used for the treatment of TRS.

In Study GO39775, 128 patients treated across several dose ranges and different schedules (80%) experienced CRS. At the time of CCOD, all but 2 CRS events had resolved, with most events resolving within 48 hours (63.0% events resolved within 24 hours, 83.4% resolved within 48 hours, and 15.6% resolved after 48 hours). Events of CRS were reversible with either standard supportive care, tocilizumab and/or steroids. Of the 128 patients with CRS events, 56 (43.8%) received tocilizumab only, 33 (25.8%) received steroids only, and 23 (18.0%) received both tocilizumab and steroids. A single patient discontinued study treatment due to a Grade 1 CRS event and encephalopathy symptoms resolving after each infusion with tocilizumab but recurring with confusion at each subsequent cycle. On 30 Aug. 2017, the FDA approved tocilizumab for the treatment of severe or life-threatening CAR T-cell-induced CRS in adults and in pediatric patients 2 years of age and older. However, recent literature supports the use of tocilizumab in all grades of CRS. Emerging evidence from the use of tocilizumab suggests patients who develop CRS may benefit from tocilizumab therapy. Specific guidance on CRS management while using tocilizumab is provided in Table 7.

TABLE 7
Tocilizumab treatment of cytokine release syndrome (CRS)

| | Pre-TCZ Tx | | Post-TCZ Treatment ^{b, c} | | | | |
|---|---|---|---|---|---|---|---|
| | (at CRS | TCZ | 6 | 1 | 2 | 3 | 8 |
| Assessment/Procedure ^{a} | onset) | Admin | hours | day | days | days | days |

| | |
|---|---|
| TCZ administration ^{d} | x |

| | | |
|---|---|---|
| Vital signs ^{e} | x ^{f} | Measure at least every 6 hours until resolution to |
| | | baseline, then every 12 hours until end of |
| | | hospitalization ^{f} |
| Pressor documentation g | x ^{f} | Record at least every 6 hours until pressors are |
| | | discontinued ^{f} |
| FiO2 | x ^{f} | Record at least every 6 hours until patient on |
| | | room air ^{f} |
| Pulse oximetry, resting | x ^{f} | Measure at least every 6 hours until resolution to |
| | | baseline, then every 12 hours until end of |
| | | hospitalization ^{f} |

Local Laboratory Assessments

| | | | | | | |
|---|---|---|---|---|---|---|
| Hematology | x | x | x | x | x | x |
| Liver function tests | x | x | x | x | x | x |
| (AST, ALT, total | | | | | | |
| bilirubin) | | | | | | |
| Serum chemistry and | x | x | x | x | x | x |
| creatinine ^{h} | | | | | | |
| CRP, LDH, and serum | x | x | x | x | x | x |
| ferritin | | | | | | |
| Coagulation (aPTT, | x | x | x | x | x | x |
| PT/INR, fibrinogen) | | | | | | |
| Infection workup ⁱ | x | | | | | |

Admin. = administration; aPTT = activated partial thromboplastin time; CRP = C-reactive protein; CRS = cytokine release syndrome; eCRF = electronic Case Report Form; FiO2 = fraction of inhaled oxygen; INR = international normalized ratio; IL-6 = interleukin 6; LDH = lactate dehydrogenase; PT = prothrombin time; TCZ = tocilizumab; Tx = treatment.
^{a} a Any assessments/procedures in Table 7 may be waived by the if the patient is hospitalized at a facility that does not have the capacity to perform such study assessments. Hospitalization should not be prolonged to perform study assessments.
^{b} If the TCZ dose is repeated, follow Table 7 following the second TCZ dose.
^{c} For post-TCZ treatment timepoints, the windows are as follows: 6 hours (±30 minutes), 1 day (24 ± 4 hours), 2 days (48 ± 4 hours), 3 days (72 ± 4 hours), and 8 days (192 ± 48 hours) after completion of TCZ infusion, respectively.
^{d} TCZ dosing: 8 mg/kg IV for patients at or above 30 kg weight; 12 mg/kg IV for patients less than 30 kg weight; repeat every 8 hours as necessary (up to a maximum of 4 doses). Doses exceeding 800 mg per infusion are not recommended.
^{e} Includes respiratory rate, heart rate, and systolic and diastolic blood pressure while the patient is in a seated or supine position, and temperature.
^{f} The maximum and minimum values for any 24-hour period should be recorded.
g Document vasopressor type and dose in the concomitant medication eCRF.
^{h} Includes sodium, potassium, chloride, bicarbonate, glucose, and blood urea nitrogen (BUN).
ⁱ Includes assessment for bacterial, fungal, and viral infections, cultures.

vi. Justification for Dose and Schedule

Ongoing Study GO39775 is investigating the escalation of the cevostamab step-up doses and TDs and both single and double step-up dosing regimens to mitigate the risk of CRS. Clinical safety and efficacy, PK and pharmacodynamic data, and PK-pharmacodynamic/exposure-response (E-R) analyses were generated on the basis of 160 patients:
For single step-up dosing, step-up doses ranging from 0.05-3.6 mg and TDs ranging from 0.15-198 mg were tested in 99 patients and 85 patients were treated with a single step-up dosing regimen 3.6 mg/TD.
The double step-up dosing regimen was evaluated in 61 patients with initial step-up doses ranging from 0.3-1.2 mg, a second dose that was kept constant at 3.6 mg, and TDs ranging from 60-160 mg and 44 patients with a double step-up dosing regimen 0.3 mg/3.6 mg/TD.
   vii. Selection of Step-Up Dose
Both single and double step-up doses effectively mitigate CRS at TDs. This mitigation of CRS risk was independent of the TD across the tested ranges (10.8-198 mg).
The 3.6 mg dose was selected as the single step-up dose and the 0.3/3.6 mg doses were selected as double step-up doses as the most effective step-up doses for mitigation of CRS risk at TD. The 0.3-mg dose is considered to be the optimal Cycle 1, Day 1 dose in the double step-up dosing regimen based on its ability to mitigate the CRS rate at the subsequent doses while also limiting the overall Cycle 1, Day 1 CRS rate and severity. In both single and double step-up dosing regimens, the 3.6-mg step-up dose was effective at limiting the frequency of CRS and Grade ≥2 CRS at the TD. Although significant E-R relationships with Grade ≥1 and Grade ≥2 CRS were observed across the step-up doses tested (0.05-3.6 mg), the 3.6 mg or 0.3/3.6 mg step-up doses were shown to adequately cap the overall acute safety risk (CRS) and maximize the safety margin for TD.
There was a trend for a lower CRS risk in the double step-up dosing regimen (0.3/3.6 mg/TD) over the single step-up dosing regimen (3.6 mg/TD) as well as a lower rate of Grade 1 CRS with symptoms in addition to fever and less Grade 2 events

In order to further optimize the dosing regimen, a split of the 3.6 mg step-up dose into 2 doses (0.3 mg on Day 1 and 3.3 mg on Day 2, 3, or 4 of Cycle 1) is being utilized in the CAMMA 2 study described herein to further mitigate CRS risk at the TD but also allow earlier therapeutic dose delivery in rapidly progressing late line participants in this study.

Quantitative Systems Pharmacology (QSP) modeling indicates that the split dosing schedule of 0.3 and 3.3 mg over consecutive days should result in a reduction in the overall Cycle 1 CRS risk compared with the 3.6 mg step-up dose in the single step-up dosing regimen. This is consistent with the dose/E-R characterization from the ongoing Study GO39775 where the Cycle 1, Day 1 dose of 0.3 mg led to a substantial reduction in the CRS risk compared with the 3.6 mg Cycle 1, Day 1 dose in the single step-up dosing regimen. This regimen also provides increased steroid premedication within 24 hours for the 3.3 mg dosing to further mitigate CRS risk.

viii. Selection of Target Dose

A TD of 160 mg has been selected for Cohorts A1 and A2 based on ongoing Study GO39775. In this study, there were no apparent E-R relationships with Grade ≥1 and Grade ≥2 CRS and Grade ≥1 immune effector cell-associated neurotoxicity syndrome (ICANS) across the tested range of TDs (0.15-198 mg). Moreover, no significant difference in the E-R relationships were observed for the other key adverse events (i.e., Grades ≥3 cytopenias, Grade ≥2 IRRs, Grade ≥2 infections, any pooled Grade ≥3 adverse events) for the single step-up and double step-up dosing regimens. A clinical dose response was observed over the range of TDs evaluated. Based on the E-R analyses, ORR and ≥VGPR rates significantly increased with an increase in exposures, with TDs≥160 mg approaching a plateau. Further evaluation of higher TDs (252 mg currently) is underway in Study GO39775 and other studies to confirm the most efficacious dose. The TD of 160 mg has been tested across more than 44 patients without exceeding the safety threshold specified in Study GO39775.

Participants enrolled in Cohorts A1 and A2 of this study will be treated with cevostamab IV in a split step-up dosing regimen 0.3/3.3 mg administered Days 1 and Days 2-4 (i.e., Day 2, Day 3, or Day 4) of Cycle 1, respectively (e.g., a split of the 3.6 mg single step-up priming regimen into 0.3 mg administered on Day 1 of Cycle 1 and on Day 2, 3, or 4 of Cycle 1), followed by the TD of 160 mg administered on Day 8 of Cycle 1 and on Day 1 of subsequent cycles every 3 weeks (Q3W). The second step-up dose (3.3 mg) will be delivered on Day 2 unless the participant experiences a CRS after the first step-up dose (0.3 mg). In this case, the second step-up dose (3.3 mg) may be delivered on Day 2, 3, or 4, upon resolution of the CRS events.

ix. Inclusion Criteria

Potential participants are eligible to be included in the study only if all of the following criteria apply:
Documented diagnosis of MM based on standard IMWG criteria (e.g., Table 6A and Table 6B).
Evidence of progressive disease based on investigator's determination of response by IMWG criteria on or after their last dosing regimen.
Prior BCMA ADC or CAR-T Cohort: participants who have received a BCMA-targeted CAR-T or ADC therapy and are triple-class refractory (i.e., refractory to at least 1 PI, 1 IMiD, and 1 anti-CD38 mAb).
Prior BCMA Bispecific Cohort: participants who have received a BCMA-targeting TDB antibody and are triple-class refractory (i.e., refractory to at least a PI, an IMiD, and an anti-CD38 mAb).
Eastern Cooperative Oncology Group (ECOG) Performance Status of 0 or 1.
Life expectancy is at least 12 weeks.
Ability to comply with the study protocol.
Agreement to protocol-specified assessments, including bone marrow biopsy and aspirate samples as detailed in the protocol.
Resolution of adverse events from prior anti-cancer therapy to Grade 51, with the following exceptions:
   Any grade alopecia is allowed
   Peripheral sensory or motor neuropathy must have resolved to Grade ≤2
Measurable disease defined as at least one of the following:
   Serum M-protein ≥0.5 g/dL (≥5 g/L)
   Urine M-protein ≥200 mg/24 hours
   sFLC assay: involved sFLCs≥10 mg/dL (≥100 mg/L) and an abnormal sFLC ratio (<0.26 or >1.65).
Laboratory values as follows:
   Hepatic function:
   AST and ALT≤2.5× upper limit of normal (ULN).
   Total bilirubin ≤1.5×ULN; participants with a documented history of Gilbert syndrome and in whom the total bilirubin elevation is ≤2.5×ULN and accompanied by elevated indirect bilirubin are eligible.
   Hematologic function (requirement within 24 hours prior to the first dose of cevostamab): Platelet count ≥75,000/mm³ without transfusion within 7 days prior to first dose (≥50,000/mm³ for participants with ≥50% bone marrow plasma cells)
   ANC≥1000/mm³.
   Total hemoglobin 28 g/dL
   Note: Participants may receive red blood cell transfusion, recombinant human erythropoietin and granulocyte colony-stimulating factor (G-CSF) to meet hematologic function eligibility criteria.
   Creatinine ≥2.0 mg/dL and creatinine clearance (CrCl) ≥30 mL/minute (either calculated using modified Cockcroft-Gault equation or per 24-hour urine collection).
   Serum calcium (corrected for albumin) level ≥11.5 mg/dL (treatment of hypercalcemia is allowed and potential participant may enroll if hypercalcemia returns to Grade ≥1 with standard treatment).
      x. Exclusion Criteria

Potential participants are excluded from the study if any of the following criteria apply:
Inability to comply with protocol-mandated hospitalization.
Prior treatment with cevostamab or another agent with the same target.
Prior BCMA ADC or CAR-T Cohort: prior treatment with any TDB antibody, including non-BCMA-targeting TDB antibody.
Prior BCMA Bispecific Cohort: treatment with TDB antibody within 12 weeks prior to enrollment in the study.
Prior use of any mAb, radioimmunoconjugate, or ADC as anti-cancer therapy within 4 weeks (12 weeks for T-cell-engaging bispecific antibodies in the Prior BCMA Bispecific Cohort or for immunotherapeutic antibodies listed below) before first study treatment, except for the use of non-myeloma therapy (e.g., denosumab for hypercalcemia).
Prior treatment with systemic immunotherapeutic agents, including but not limited to, cytokine therapy and anti-CTLA-4, anti-PD-1, and anti-PD-L1 therapeutic antibodies within 12 weeks or 5 half-lives of the drug, whichever is shorter, before first study treatment.
Prior treatment with CAR-T cell therapy within 12 weeks before first cevostamab infusion.
Known treatment-related, immune-mediated adverse events associated with prior checkpoint inhibitors as follows:
   Prior PD-L1/PD-1 or CTLA-4 inhibitor: Grade ≥3 adverse events with the exception of Grade 3 endocrinopathy managed with replacement therapy.
   Grade 1-2 adverse events that did not resolve to baseline after treatment discontinuation.
Treatment with radiotherapy, any chemotherapeutic agent, or treatment with any other anti-cancer agent (investigational or otherwise) within 4 weeks or 5 half-lives of the drug, whichever is shorter, prior to first study treatment.
Autologous stem cell transplantation (SCT) within 100 days prior to first study treatment.
Prior allogeneic SCT.
Circulating plasma cell count exceeding 500/μL or 5% of the peripheral blood white cells.
Prior solid organ transplantation.
History of autoimmune disease, including but not limited to, myasthenia gravis, myositis, autoimmune hepatitis, systemic lupus erythematosus, rheumatoid arthritis, inflammatory bowel disease, vascular thrombosis associated with antiphospholipid syndrome, Wegener's granulomatosis, Sjögren's syndrome, Guillain-Barré syndrome, multiple sclerosis, vasculitis, or glomerulonephritis Participants with a history of autoimmune-related hypothyroidism on a stable dose of thyroid replacement hormone may be eligible for this study.
History of confirmed progressive multifocal leukoencephalopathy.
History of severe allergic or anaphylactic reactions to mAb therapy (or recombinant antibody-related fusion proteins).
Known history of amyloidosis (e.g., positive Congo Red stain or equivalent in tissue biopsy).
Lesions in proximity of vital organs that may develop sudden decompensation/deterioration in the setting of a tumor flare.
History of other malignancy within 2 years prior to screening, except those with negligible risk of metastasis or death (e.g., 5-year OS>90%), such as ductal carcinoma in situ not requiring chemotherapy, appropriately treated carcinoma in situ of the cervix, non-melanoma skin carcinoma, low-grade, localized prostate cancer (Gleason score ≤7) not requiring treatment or appropriately treated Stage I uterine cancer.
Current or past history of CNS disease, such as stroke, epilepsy, CNS vasculitis, neurodegenerative disease, or CNS involvement by MM.
   Participants with a history of stroke who have not experienced a stroke or transient ischemic attack in the past 2 years and have no residual neurologic deficits, as judged by the investigator, are allowed.
   Participants with a history of epilepsy who have had no seizures in the past 2 years while not receiving any anti-epileptic medications are allowed.
Significant cardiovascular disease (such as, but not limited to, New York Heart Association Class III or IV cardiac disease, myocardial infarction within the last 6 months, uncontrolled arrhythmias, or unstable angina) that may limit a potential participant's ability to adequately respond to a CRS event.
Symptomatic active pulmonary disease or requiring supplemental oxygen.
Known active bacterial, viral, fungal, mycobacterial, parasitic, or other infection (excluding fungal infections of nail beds) at study enrollment, or any major episode of infection requiring treatment with IV antimicrobials where the last dose of an IV antimicrobial was given within 14 days prior to first study treatment.
Active symptomatic COVID-19 infection at study enrollment or requiring treatment with IV antiviral where the last dose of IV antiviral treatment was given within 14 days prior to first study treatment. Patients with active COVID-19 infection must have clinical recovery and two negative antigen tests at least 24 hours apart prior to first study treatment.
   Primary prophylaxis for COVID-19 is not considered treatment for COVID-19 infection.
Positive and quantifiable Epstein-Barr virus (EBV) PCR or cytomegalovirus (CMV) PCR prior to first study treatment.
Known or suspected chronic active EBV infection.
Known history of Grade ≥CRS or immune effector cell-associated neurotoxicity syndrome (ICANS) with prior bispecific therapies.
Known history of HLH or MAS.
Recent major surgery within 4 weeks prior to first study treatment.
   Protocol-mandated procedures (e.g., bone marrow biopsies) are permitted.
Positive serologic or polymerase chain reaction (PCR) test results for acute or chronic hepatitis B virus (HBV) infection.
   Participants whose HBV infection status cannot be determined by serologic test results (www.cdc.gov/hepatitis/hbv/pdfs/serologicchartv8.pdf) must be negative for HBV by PCR to be eligible for study participation.
Acute or chronic hepatitis C virus (HCV) infection.
   Participants who are positive for HCV antibody must be negative for HCV by PCR to be eligible for study participation.
Known history of HIV seropositivity.
Administration of a live, attenuated vaccine within 4 weeks before first study treatment or anticipation that such a live attenuated vaccine will be required during the study.
   Influenza vaccination may be given during influenza season (approximately October to May in the Northern Hemisphere; approximately May to October in the Southern Hemisphere). Participants must not receive live, attenuated influenza vaccines (e.g., FLUMIST®) at any time during the study treatment period.
   Severe acute respiratory syndrome coronavirus 2 (SARS-CoV-2) vaccines, may be given in accordance with the approved/authorized vaccine label and official/local immunization guidance. SARS-CoV-2 vaccines must not be administered within 1 week before first study treatment or during Cycle 1.
   Investigators should review the vaccination status of potential study participants being considered for this study and follow the local guidelines, e.g., U.S. Centers for Disease Control and Prevention guidelines, for adult vaccination with any other non-live vaccines intended to prevent infectious diseases prior to study.
Treatment with systemic immunosuppressive medications (including, but not limited to, cyclophosphamide, azathioprine, methotrexate, thalidomide, and anti-TNF agents), with the exception of corticosteroid treatment ≤10 mg/day prednisone or equivalent, within 2 weeks prior to first study treatment.
   The use of inhaled corticosteroids is permitted.
   The use of mineralocorticoids for management of orthostatic hypotension is permitted.
   The use of physiologic doses of corticosteroids for management of adrenal insufficiency is permitted.
History of illicit drug or alcohol abuse within 12 months prior to screening, in the investigator's judgment.
Any medical condition or abnormality in clinical laboratory tests that, in the investigator's judgment, precludes the participant's safe participation in and completion of the study, or which could affect compliance with the protocol or interpretation of results.
   xi. End of Study Definition and Duration of Participation

The end of this study is defined as the date of the last visit of the last participant in the study the date at which the last data point required for statistical analysis or safety follow-up is received from the last participant, whichever occurs later.

Treatment will continue until disease progression per IMWG criteria, unacceptable toxicity, withdrawal of consent or death, whichever occurs first. The total duration of study is expected to be 2 years after the LPI or all participants have completed treatment (or discontinued or withdrew from the study before completing treatment).

xii. Premedication

Corticosteroid premedication (dexamethasone preferred, alternative corticosteroid equivalent such as methylprednisolone 80 mg IV is also acceptable) may be administered prior to the administration of each cevostamab dose as follows:
Cycle 1
   Dose 1 (0.3 mg): give dexamethasone 20 mg IV 1 hour (±15 minutes) prior to cevostamab infusion.
   Dose 2 (3.3 mg): give dexamethasone 20 mg IV 1 hour (±15 minutes) prior to cevostamab infusion.
   TD: give dexamethasone 20 mg oral approximately 24 hours prior and dexamethasone 20 mg IV 1 hour (±15 minutes) prior to cevostamab infusion.
Cycle 2
   TD: give dexamethasone 20 mg IV 1 hour (±15 minutes) prior to cevostamab infusion.
In Cycles 3 and beyond: only if the participant experienced CRS with the prior dose.

In addition, premedication with oral acetaminophen or paracetamol (e.g., 500-1000 mg) and 25-50 mg diphenhydramine must be administered prior to administration of all cevostamab doses, unless contraindicated. For sites that do not have access to diphenhydramine, an equivalent medication may be substituted per local practice.

Participants with elevated uric acid levels prior to cevostamab treatment or who are considered to be at high risk for tumor lysis syndrome (TLS) will receive prophylaxis for TLS prior to each cevostamab infusion during Cycle 1. Prophylaxis guidelines include the following:
Hydration, consisting of a fluid intake of approximately 2-3 L/day starting 24-48 hours prior to the first dose of cevostamab; followed by IV hydration at a rate of 125-200 mL/hour beginning at the conclusion of Cycle 1 infusions of cevostamab and continued for at least 24 hours thereafter. Modification of fluid rate should be considered for participants with specific medical needs.
Administration of an agent to reduce uric acid.
   ELITEK® (rasburicase) 0.2 mg/kg IV over 30 minutes prior to the first dose cevostamab and daily for up to 5 days thereafter should be administered, unless contraindicated (Rasburicase USPI).
   Treatment with rasburicase should continue as specified above, or if laboratory evidence of TLS is observed until normalization of serum uric acid or other laboratory parameters.

### SEQUENCE LISTING

Table 8 shows sequences that are used throughout the application.

TABLE 8
Sequence Listing

| SEQ ID NO: | SEQUENCE |
|---|---|
| SEQ ID NO: 1 | RFGVH |
| SEQ ID NO: 2 | VIWRGGSTDYNAAFVS |
| SEQ ID NO: 3 | HYYGSSDYALDN |
| SEQ ID NO: 4 | KASQDVRNLVV |
| SEQ ID NO: 5 | SGSYRYS |
| SEQ ID NO: 6 | QQHYSPPYT |
| SEQ ID NO: 7 | EVQLVESGPGLVKPSETLSLTCTVSGFSLTRFGVHWVRQPPGKGLEWLGVIWRGGS |
| | TDYNAAFVSRLTISKDNSKNQVSLKLSSVTAADTAVYYCSNHYYGSSDYALDNWGQG |
| | TLVTVSS |
| SEQ ID NO: 8 | DIQMTQSPSSLSASVGDRVTITCKASQDVRNLVVWFQQKPGKAPKLLIYSGSYRYSG |
| | VPSRFSGSGSGTDFTLTISSLOPEDFATYYCQQHYSPPYTFGQGTKVEIK |
| SEQ ID NO: 9 | SYYIH |
| SEQ ID NO: 10 | WIYPENDNTKYNEKFKD |
| SEQ ID NO: 11 | DGYSRYYFDY |
| SEQ ID NO: 12 | KSSQSLLNSRTRKNYLA |
| SEQ ID NO: 13 | WTSTRKS |
| SEQ ID NO: 14 | KQSFILRT |
| SEQ ID NO: 15 | EVQLVQSGAEVKKPGASVKVSCKASGFTFTSYYIHWVRQAPGQGLEWIGWIYPEND |
| | NTKYNEKFKDRVTITADTSTSTAYLELSSLRSEDTAVYYCARDGYSRYYFDYWGQGT |
| | LVTVSS |
| SEQ ID NO: 16 | DIVMTQSPDSLAVSLGERATINCKSSQSLLNSRTRKNYLAWYQQKPGQSPKLLIYWTS |
| | TRKSGVPDRFSGSGSGTDFTLTISSLQAEDVAVYYCKQSFILRTFGQGTKVEIK |
| SEQ ID NO: 17 | EVQLVESGPGLVKPSETLSLTCTVSGFSLT |
| SEQ ID NO: 18 | WVRQPPGKGLEWLG |
| SEQ ID NO: 19 | RLTISKDNSKNQVSLKLSSVTAADTAVYYCSN |
| SEQ ID NO: 20 | WGQGTLVTVSS |
| SEQ ID NO: 21 | DIQMTQSPSSLSASVGDRVTITC |
| SEQ ID NO: 22 | WFQQKPGKAPKLLIY |
| SEQ ID NO: 23 | GVPSRFSGSGSGTDFTLTISSLQPEDFATYYC |
| SEQ ID NO: 24 | FGQGTKVEIK |
| SEQ ID NO: 25 | EVQLVQSGAEVKKPGASVKVSCKASGFTFT |
| SEQ ID NO: 26 | WVRQAPGQGLEWIG |
| SEQ ID NO: 27 | RVTITADTSTSTAYLELSSLRSEDTAVYYCAR |
| SEQ ID NO: 28 | WGQGTLVTVSS |
| SEQ ID NO: 29 | DIVMTQSPDSLAVSLGERATINC |
| SEQ ID NO: 30 | WYQQKPGQSPKLLIY |
| SEQ ID NO: 31 | GVPDRFSGSGSGTDFTLTISSLQAEDVAVYYC |
| SEQ ID NO: 32 | FGQGTKVEIK |
| SEQ ID NO: 33 | gcagtttcagaacccagccagcctctctcttgctgcctagcctcctgccggcctcatcttcgcccagccaaccccgcctgg |
| | agccctatggccaactgcgagttcagcccggtgtccggggacaaaccctgctgccggctctctaggagagcccaactct |
| | gtcttggcgtcagtatcctggtcctgatcctcgtcgtggtgctcgcggtggtcgtcccgaggtggcgccagcagtggagcg |
| | gtccgggcaccaccaagcgctttcccgagaccgtcctggcgcgatgcgtcaagtacactgaaattcatcctgagatgag |
| | acatgtagactgccaaagtgtatgggatgctttcaagggtgcatttatttcaaaacatccttgcaacattactgaagaagac |
| | tatcagccactaatgaagttgggaactcagaccgtaccttgcaacaagattcttctttggagcagaataaaagatctggcc |
| | catcagttcacacaggtccagcgggacatgttcaccctggaggacacgctgctaggctaccttgctgatgacctcacatg |
| | gtgtggtgaattcaacacttccaaaataaactatcaatcttgcccagactggagaaaggactgcagcaacaaccctgttt |
| | cagtattctggaaaacggtttcccgcaggtttgcagaagctgcctgtgatgtggtccatgtgatgctcaatggatcccgcag |
| | taaaatctttgacaaaaacagcacttttgggagtgtggaagtccataatttgcaaccagagaaggttcagacactagagg |
| | cctgggtgatacatggtggaagagaagattccagagacttatgccaggatcccaccataaaagagctggaatcgattat |
| | aagcaaaaggaatattcaattttcctgcaagaatatctacagacctgacaagtttcttcagtgtgtgaaaaatcctgaggat |
| | tcatcttgcacatctgagatctgagccagtcgctgtggttgttttagctccttgactccttgtggtttatgtcatcatacatgactca |
| | gcatacctgctggtgcagagctgaagattttggagggtcctccacaataaggtcaatgccagagacggaagcctttttcc |
| | ccaaagtcttaaaataacttatatcatcagcatacctttattgtgatctatcaatagtcaagaaaaattattgtataagattaga |
| | atgaaaattgtatgttaagttacttcactttaattctcatgtgatccttttatgttatttatatattggtaacatcctttctattgaaaaat |
| | caccacaccaaacctctcttattagaacaggcaagtgaagaaaagtgaatgctcaagtttttcagaaagcattacatttcc |
| | aaatgaatgaccttgttgcatgatgtatttttgtacccttcctacagatagtcaaaccataaacttcatggtcatgggtcatgttg |
| | gtgaaaattattctgtaggatataagctacccacgtacttggtgctttaccccaacccttccaacagtgctgtgaggttggtat |
| | tatttcattttttagatgagaaaatgggagctcagagaggttatatatttaagttggtgcaaaagtaattgcaagttttgccacc |
| | gaaaggaatggcaaaaccacaattatttttgaaccaacctaataatttaccgtaagtcctacatttagtatcaagctagag |
| | actgaatttgaactcaactctgtccaactccaaaattcatgtgctttttccttctaggcctttcataccaaactaatagtagtttat |
| | attctcttccaacaaatgcatattggattaaattgactagaatggaatctggaatatagttcttctggatggctccaaaacac |
| | atgtttttcttcccccgtcttcctcctcctcttcatgctcagtgttttatatatgtagtatacagttaaaatatacttgttgctggtactg |
| | gcagcttatattttctctcttttttcatggattaaccttgcttgagggctttaacaattgtattactttttcaaagaactaagctttagct |
| | tcattgatttttttctatttaattgggttttgctcttctctttagcattggaaacatagaaatgctttctgatttctttgggtagatttacgt |
| | attcagcttcttgagatggaagtttagatcactgatccttcagcttgttttcttttttgtatacatagattttaggacgatatattttcc |
| | cttgagttctgctttagctgcagctcttatgttttgatatgcctctctttattatccttcagttaaaaatatctttcaattcattgttatata |
| | aaaatatgtgcctagtttttaacatctggagattttctagttttgaaaaaaacataagccaggcatggtggctcacacctgtat |
| | ccccagcactttgggaggccgagacgggaggatcgcctgagctcaggagtttttacaccagcctgggaataacagtga |
| | gacattatctccaaaaaaattacctgggtatggtgttgtgcacctgtagtcccagctactctggagactgaggtgggaggat |
| | tgtttgagcttgggaggttgaggctgcagggagctgtgatcacaccactgcactctggcctgagtgacagattgagaccct |
| | gtctcaataaaagcaaaaataaagaaaataaaccatatgtgttgaacaaaggattaataaattaatttgagactccttca |
| | gggaatgaccacaatttattgaaaatagcctaaatgttggagtcaggcatttctggattcatattttgacatcatgctgtcatct |
| | tgaacaaaatgcctaacctttctgaacttcaacttccttgccactcaaataaggattacaaaacttaaaatgtggtaagtact |
| | aaagacgacagcaaaaattgagtccagcacagagcttcctaaataagcaagcactcaacagagttggttcctttcttcct |
| | cccctgcttgacaatccagtttcccacaggagcctttgtagctgtagccaccatggtcagtccagggattcttcactagccc |
| | cttctcccctggcagacatccttgtgggagtttagtcttggctcgacatgaggatgggggtttgggaccagttctgagtgaga |
| | atcagacttgccccaagttgccattagctccccctgcagaatgtcttcagaatcggggcccggtcagtctcctgggtgacct |
| | gctgttttcctcttaagatcctttccactttggttgctgctttcgggactcatcgagtccttgctcaacaggataccccttgaagtg |
| | gctgcctgggccacatccccttccaaacaagaaatcaaaatattagaaatcaatttttgaaatttcccctaggaagactcat |
| | ttgagtgttcaagttcagagccagtggagaccttaggggagggtggtcacaaggattttgcacagtgctttagagggtccc |
| | agggagccacagaggtggtgaggggctgggtgctcttttctccgtgcatgaccttgtgtgtctatcttcattaccacaatgcc |
| | tcatctctacctcctttccccctgtagttccaacgtgggtatctttgccatctctggcccgaaggactttctgacctacatgtata |
| | aataccccctcacaatatatattacttttcctataagtgacttctctactggattactggttgctcatacacctcatattttactcgt |
| | aaatctactactccctgtctgcctactccattctcatttgctgtagaaaattctcttaccatcccaactttcacccaccatcatgct |
| | tacccaaaggctgtgggaatgacctgggccctaatgccccttttctaaattcctaaggctcaccattttcctattgtaatggttc |
| | ttgaccttataatgtttgaggcaccttttcaaatatagtcctttgatttcagactgaatacttgaaaggacacacacacacata |
| | cgtaagtgcatatgactgcatacacccacacacacacacgtgcctgtatacagtcatatgatacatacacaaacacacg |
| | cacacaagcctgcatacatcatatgccaacagtggggatatgttctgagaaatgcatcattagatgattttgtcattgtgtga |
| | acatcatagagtgtacttacactaacctagatggtctaacctactacacacccaggctacatggtatcacctattcctccta |
| | ggctacaagcctgtacagcgtgtgtctgtactaaatgctgtgggcaattttaacctgatggtaaatgtttgtgtatctaaacat |
| | atctaaacatagaaaaggtacagtaaacatgcagtattataatcttatgagaccgtcatcatatatgtggtccactgtttggg |
| | ccatcattggctgaaaagtggttatgcgacacatgactgtatatatactttcctgttacaacaacagtgtctctcaatccaca |
| | gtaattgcagcatccagtaggtcttactttagccctgagtcaccatttgtgtcaacgtgtttagtgccatgtccacgtctctcatg |
| | taactggcagagctatcaaatattttggcaaaacacattgtttctttggctttgccttggtaactttctgtgccttttgtagctcttgtt |
| | tggaagaagctcaacccatgtctgcacactgtgatacaagggggacagcatcgacatcgacttacttcttggtgccttattc |
| | ctccttagaacaattcctaaatctgtaacttaagtttctcaggaagattccatactgcacagaaaactgcttttgtgggtttttaa |
| | aaggcaagttgttatatgtgctggatagtttttaagtatgacataaaaattgtataaagtaaaatattaaaatacacctagaat |
| | actgtataactttaagtcattttatcaacacattgctaatccagatattttcccgcagttttttttgaataacagagcaattaattt |
| | acttttactatgaagagtcatcattttagtatgtattttaagcaatccaccaagaactcagtaggcagctgagaggtgctgcc |
| | cagagaagtggtgattagcttggccttagctcacccacacaaagcacaacaggctttgaactattccctaacggggcattt |
| | attcttttttttttttttttttgggagacggagtctcgctgtcgcccaggctagagtgcagtggcgcgatctcggctcactgcagg |
| | ctccaccccctggggttcacgccattctcctgcctcagcctcccaagtagctgggactgcaggcgcccgccatctcgccc |
| | ggctaattttttgtatttttagtagagacggggtttcaccgtgttagccaggatagggcatttattcttgaacttgattcagagag |
| | gcacacattaccattctctaatcagaatgcaagtagcgcaaggcggtggaaactatggaattcggaggcaggtgatgca |
| | ttgggcgagtttattaacatctgtgactctctagtttgaaatttatttgtaacagacaaaaatgaattaaacaaacaataaaa |
| | gtataataaagaa |
| SEQ ID NO: 34 | MANCEFSPVSGDKPCCRLSRRAQLCLGVSILVLILVVVLAVVVPRWRQQWSGPGTTK |
| | RFPETVLARCVKYTEIHPEMRHVDCQSVWDAFKGAFISKHPCNITEEDYQPLMKLGT |
| | QTVPCNKILLWSRIKDLAHQFTQVQRDMFTLEDTLLGYLADDLTWCGEFNTSKINYQS |
| | CPDWRKDCSNNPVSVFWKTVSRRFAEAACDVVHVMLNGSRSKIFDKNSTFGSVEVH |
| | NLQPEKVQTLEAWVIHGGREDSRDLCQDPTIKELESIISKRNIQFSCKNIYRPDKFLQC |
| | VKNPEDSSCTSEI |
| SEQ ID NO: 35 | EVQLVESGPGLVKPSETLSLTCTVSGFSLTRFGVHWVRQPPGKGLEWLGVIWRGGS |
| | TDYNAAFVSRLTISKDNSKNQVSLKLSSVTAADTAVYYCSNHYYGSSDYALDNWGQG |
| | TLVTVSSASTKGPSVFPLAPSSKSTSGGTAALGCLVKDYFPEPVTVSWNSGALTSGV |
| | HTFPAVLQSSGLYSLSSVVTVPSSSLGTQTYICNVNHKPSNTKVDKKVEPKSCDKTHT |
| | CPPCPAPELLGGPSVFLFPPKPKDTLMISRTPEVTCVVVDVSHEDPEVKFNWYVDGV |
| | EVHNAKTKPREEQYGSTYRVVSVLTVLHQDWLNGKEYKCKVSNKALPAPIEKTISKAK |
| | GQPREPQVYTLPPSREEMTKNQVSLWCLVKGFYPSDIAVEWESNGQPENNYKTTPP |
| | VLDSDGSFFLYSKLTVDKSRWQQGNVFSCSVMHEALHNHYTQKSLSLSPGK |
| SEQ ID NO: 36 | DIQMTQSPSSLSASVGDRVTITCKASQDVRNLVVWFQQKPGKAPKLLIYSGSYRYSG |
| | VPSRFSGSGSGTDFTLTISSLQPEDFATYYCQQHYSPPYTFGQGTKVEIKRTVAAPSV |
| | FIFPPSDEQLKSGTASVVCLLNNFYPREAKVQWKVDNALQSGNSQESVTEQDSKDST |
| | YSLSSTLTLSKADYEKHKVYACEVTHQGLSSPVTKSFNRGEC |
| SEQ ID NO: 37 | EVQLVQSGAEVKKPGASVKVSCKASGFTFTSYYIHWVRQAPGQGLEWIGWIYPEND |
| | NTKYNEKFKDRVTITADTSTSTAYLELSSLRSEDTAVYYCARDGYSRYYFDYWGQGT |
| | LVTVSSASTKGPSVFPLAPSSKSTSGGTAALGCLVKDYFPEPVTVSWNSGALTSGVH |
| | TFPAVLQSSGLYSLSSVVTVPSSSLGTQTYICNVNHKPSNTKVDKKVEPKSCDKTHTC |
| | PPCPAPELLGGPSVFLFPPKPKDTLMISRTPEVTCVVVDVSHEDPEVKFNWYVDGVE |
| | VHNAKTKPREEQYGSTYRVVSVLTVLHQDWLNGKEYKCKVSNKALPAPIEKTISKAKG |
| | QPREPQVYTLPPSREEMTKNQVSLSCAVKGFYPSDIAVEWESNGQPENNYKTTPPVL |
| | DSDGSFFLVSKLTVDKSRWQQGNVFSCSVMHEALHNHYTQKSLSLSPGK |
| SEQ ID NO: 38 | DIVMTQSPDSLAVSLGERATINCKSSQSLLNSRTRKNYLAWYQQKPGQSPKLLIYWTS |
| | TRKSGVPDRFSGSGSGTDFTLTISSLQAEDVAVYYCKQSFILRTFGQGTKVEIKRTVA |
| | APSVFIFPPSDEQLKSGTASVVCLLNNFYPREAKVQWKVDNALQSGNSQESVTEQDS |
| | KDSTYSLSSTLTLSKADYEKHKVYACEVTHQGLSSPVTKSFNRGEC |

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1-101. (canceled)

102. A method of treating a subject having a relapsed or refractory (R/R) multiple myeloma (MM), the method comprising administering to the subject a bispecific antibody that binds to Fc receptor-homolog 5 (FcRH5) and cluster of differentiation 3 (CD3) in a dosing regimen comprising at least a first 21-day dosing cycle (C1), wherein the first 21-day dosing cycle comprises a first dose (C1D1), a second dose (C1D2), and a third dose (C1D3) of the bispecific antibody, wherein the C1D1 is between about 0.2 mg to about 0.4 mg and is administered to the subject on Day 1 of the first dosing cycle, the C1D2 is about 3.1 mg to about 3.4 mg and is administered to the subject on Day 2, Day 3, or Day 4 of the first dosing cycle, and the C1D3 is greater than the C1D2, wherein the bispecific antibody that binds to FcRH5 and CD3 comprises an anti-FcRH5 arm comprising a first binding domain comprising the following six hypervariable regions (HVRs):
(i) an HVR-H1 comprising the amino acid sequence of RFGVH (SEQ ID NO: 1);
(ii) an HVR-H2 comprising the amino acid sequence of VIWRGGSTDYNAAFVS (SEQ ID NO: 2);
(iii) an HVR-H3 comprising the amino acid sequence of HYYGSSDYALDN (SEQ ID NO:3);
(iv) an HVR-L1 comprising the amino acid sequence of KASQDVRNLVV (SEQ ID NO: 4);
(v) an HVR-L2 comprising the amino acid sequence of SGSYRYS (SEQ ID NO: 5); and
(vi) an HVR-L3 comprising the amino acid sequence of QQHYSPPYT (SEQ ID NO: 6), and an anti-CD3 arm comprising a second binding domain comprising the following six HVRs:
(i) an HVR-H1 comprising the amino acid sequence of SYYIH (SEQ ID NO: 9);
(ii) an HVR-H2 comprising the amino acid sequence of WIYPENDNTKYNEKFKD (SEQ ID NO: 10);
(iii) an HVR-H3 comprising the amino acid sequence of DGYSRYYFDY (SEQ ID NO: 11);
(iv) an HVR-L1 comprising the amino acid sequence of KSSQSLLNSRTRKNYLA (SEQ ID NO: 12);
(v) an HVR-L2 comprising the amino acid sequence of WTSTRKS (SEQ ID NO: 13); and
(vi) an HVR-L3 comprising the amino acid sequence of KQSFILRT (SEQ ID NO: 14).

103. The method of claim 102, wherein the C1D3 is between about 20 mg to about 600 mg and is administered to the subject on Day 8 of the C1, on Day 9 of the C1, or after Day 9 of the C1.

104. The method of claim 103, wherein the C1D3 is about 160 mg.

105. The method of claim 102, wherein the method further comprises one or more additional 21-day dosing cycles, wherein the bispecific antibody that binds to FcRH5 and CD3 is administered on or about Day 1 of the one or more additional 21-day dosing cycles until the subject experiences disease progression, unacceptable toxicity, or death.

106. The method of claim 105, wherein the bispecific antibody that binds to FcRH5 and CD3 is administered at a dose of about 160 mg on or about Day 1 of the one or more additional 21-day dosing cycles until the subject experiences disease progression, unacceptable toxicity, or death.

107. The method of claim 102, wherein:
(a) the first binding domain of the anti-FcRH5 arm comprises:
(i) a heavy chain variable (VH) domain comprising an amino acid sequence having at least 95% sequence identity to the amino acid sequence of SEQ ID NO: 7;
(ii) a light chain variable (VL) domain comprising an amino acid sequence having at least 95% sequence identity to the amino acid sequence of SEQ ID NO: 8; or
(iii) a VH domain as in (a) and a VL domain as in (b); and/or
(b) the second binding domain of the anti-CD3 arm comprises:
(i) a VH domain comprising an amino acid sequence having at least 95% sequence identity to the amino acid sequence of SEQ ID NO: 15;
(ii) a VL domain comprising an amino acid sequence having at least 95% sequence identity to the amino acid sequence of SEQ ID NO: 16; or
(iii) a VH domain as in (a) and a VL domain as in (b).

108. The method of claim 107, wherein:
(a) the first binding domain comprises a VH domain comprising an amino acid sequence of SEQ ID NO: 7 and a VL domain comprising an amino acid sequence of SEQ ID NO: 8;
(b) the second binding domain comprises a VH domain comprising an amino acid sequence of SEQ ID NO: 15 and a VL domain comprising an amino acid sequence of SEQ ID NO: 16;
(c) the anti-FcRH5 arm comprises a heavy chain polypeptide (H1) and a light chain polypeptide (L1) and the anti-CD3 arm comprises a heavy chain polypeptide (H2) and a light chain polypeptide (L2), wherein:
(i) H1 comprises the amino acid sequence of SEQ ID NO: 35,
(ii) L1 comprises the amino acid sequence of SEQ ID NO: 36,
(iii) H2 comprises the amino acid sequence of SEQ ID NO: 37, and
(iv) L2 comprises the amino acid sequence of SEQ ID NO: 38; and/or
(d) the bispecific antibody that binds to FcRH5 and CD3 is cevostamab.

109. The method of claim 102, wherein:
(a) the bispecific antibody that binds to FcRH5 and CD3 comprises an aglycosylation site mutation; and/or
(b) the bispecific antibody is a monoclonal antibody, a humanized antibody, a chimeric antibody, or an antibody fragment that binds FcRH5 and CD3.

110. The method of claim 109, wherein:
(a) the aglycosylation site mutation reduces effector function of the bispecific antibody;
(b) the aglycosylation site mutation is a substitution mutation; and/or
(c) the antibody fragment is selected from the group consisting of Fab, Fab′-SH, Fv, scFv, and (Fab′)₂ fragments.

111. The method of claim 102, wherein:
(a) the bispecific antibody that binds to FcRH5 and CD3 is a full-length antibody;
(b) the bispecific antibody is an IgG antibody; and/or
(c) the bispecific antibody comprises one or more heavy chain constant domains, wherein the one or more heavy chain constant domains are selected from a first CH1 (CH1₁) domain, a first CH2 (CH2₁) domain, a first CH3 (CH3₁) domain, a second CH1 (CH1₂) domain, second CH2 (CH2₂) domain, and a second CH3 (CH3₂) domain.

112. The method of claim 111, wherein:
(a) the IgG antibody is an IgG1 antibody;
(b) at least one of the one or more heavy chain constant domains is paired with another heavy chain constant domain;
(c) the CH3₁ and CH3₂ domains each comprise a protuberance or cavity, and wherein the protuberance or cavity in the CH3₁ domain is positionable in the cavity or protuberance, respectively, in the CH3₂ domain; and/or
(d) the CH2₁ and CH2₂ domains each comprise a protuberance or cavity, and wherein the protuberance or cavity in the CH2₁ domain is positionable in the cavity or protuberance, respectively, in the CH2₂ domain.

113. The method of claim 112, wherein:
(a) the CH3₁ and CH3₂ domains meet at an interface between the protuberance and cavity; and/or
(b) the CH2₁ and CH2₂ domains meet at an interface between said protuberance and cavity, wherein the anti-FcRH5 arm comprises the protuberance and the anti-CD3 arm comprises the cavity.

114. The method of claim 113, wherein a CH3 domain of the anti-FcRH5 arm comprises a protuberance comprising a T366W amino acid substitution mutation (EU numbering) and a CH3 domain of the anti-CD3 arm comprises a cavity comprising T366S, L368A, and Y407V amino acid substitution mutations (EU numbering).

115. The method of claim 102, wherein:
(a) the bispecific antibody that binds to FcRH5 and CD3 is administered to the subject concurrently with one or more additional therapeutic agents;
(b) the bispecific antibody that binds to FcRH5 and CD3 is administered to the subject prior to administration of one or more additional therapeutic agents; or
(c) the bispecific antibody that binds to FcRH5 and CD3 is administered to the subject subsequent to administration of one or more additional therapeutic agents.

116. The method of claim 115, wherein the one or more additional therapeutic agents comprise:
(a) an effective amount of tocilizumab;
(b) an effective amount of a BCMA-directed therapeutic agent;
(c) an effective amount of acetaminophen or paracetamol; and/or
(d) an effective amount of diphenhydramine.

117. The method of claim 116, wherein:
(a) tocilizumab is administered to the subject by intravenous infusion;
(b) the subject weighs:
(i) ≥30 kg, and tocilizumab is administered to the subject at a dose of 8 mg/kg; or
(ii) <30 kg, and tocilizumab is administered to the subject at a dose of 12 mg/kg,
wherein the tocilizumab is administered to the subject at a dose that does not exceed 800 mg; and/or
(c) tocilizumab is administered to the subject 2 hours before administration of the bispecific antibody.

118. The method of claim 102, wherein the subject has a CRS event, and the method further comprises treating the symptoms of the CRS event while suspending treatment with the bispecific antibody that binds to FcRH5 and CD3, wherein treating the symptoms of the CRS event comprises intravenously administering to the subject a single dose of about 8 mg/kg.

119. The method of claim 118, wherein the CRS event does not resolve or worsens within 24 hours of treating the symptoms of the CRS event, and the method further comprises administering to the subject one or more additional doses of tocilizumab to manage the CRS event, wherein the one or more additional doses of tocilizumab are administered intravenously to the subject at a dose of about 8 mg/kg.

120. The method of claim 116, wherein:
(a) the acetaminophen or paracetamol is administered to the subject orally at a dose of between about 500 mg to about 1000 mg; and/or
(b) the diphenhydramine is administered to the subject orally at a dose of between about 25 mg to about 50 mg.

121. The method of claim 102, wherein:
(a) the method comprises pre-medication with the following agents prior to administration of the bispecific antibody to the subject:
(i) a corticosteroid;
(ii) acetaminophen or paracetamol, and/or
(iii) diphenhydramine;
(b) the subject has received at least four prior lines of treatment for the MM; and/or
(c) the subject has been exposed to a prior treatment comprising a proteasome inhibitor (PI), an IMiD, an anti-CD38 therapeutic agent, and/or an autologous stem cell transplant (ASCT).

122. The method of claim 121, wherein:
(a) the corticosteroid is administered to the subject:
(i) 1 hour (±15 minutes) prior to any administration of the bispecific antibody during the first phase, or
(ii) 24 hours prior to any administration of the bispecific antibody during the first phase;
(b) the subject has experienced CRS with a prior administration of the bispecific antibody and the corticosteroid is administered to the subject 1 hour (±15 minutes) prior to any administration of the bispecific antibody during the second phase;
(c) the corticosteroid is dexamethasone or methylprednisolone;
(d) the corticosteroid is administered to the subject intravenously;
(e) the acetaminophen or paracetamol is administered to the subject orally at a dose of between 500 mg to 1000 mg;
(f) the diphenhydramine is administered to the subject orally at a dose of between 25 mg to 50 mg;
(g) the PI is bortezomib, carfilzomib, or ixazomib;
(h) the IMiD is thalidomide, lenalidomide, or pomalidomide; and/or
(i) the anti-CD38 therapeutic agent is an anti-CD38 antibody.

123. The method of claim 122, wherein:
(a) the dexamethasone is administered to the subject at a dose of about 20 mg or the methylprednisolone is administered to the subject at a dose of about 80 mg; and/or
(b) the anti-CD38 antibody is daratumumab, MOR202, or isatuximab.

124. A method of treating a subject having a relapsed or refractory (R/R) multiple myeloma (MM), wherein the subject has previously received a B cell maturation factor (BCMA)-targeting therapeutic agent, the method comprising administering to the subject a bispecific antibody that binds to Fc receptor-homolog 5 (FcRH5) and cluster of differentiation 3 (CD3) in a dosing regimen comprising:
(a) a first phase comprising administering the bispecific antibody to the subject in at least a first 21-day dosing cycle (C1), wherein the first phase comprises administration of the bispecific antibody to the subject on (i) Day 1 of the C1; and (ii) Day 2, Day 3, or Day 4 of the C1; and
(b) a second phase comprising one or more 21-day dosing cycles, wherein the second phase comprises administering the bispecific antibody to the subject every three weeks (Q3W),
wherein the bispecific antibody that binds to FcRH5 and CD3 comprises an anti-FcRH5 arm comprising a first binding domain comprising the following six hypervariable regions (HVRs):
(a) an HVR-H1 comprising the amino acid sequence of RFGVH (SEQ ID NO: 1);
(b) an HVR-H2 comprising the amino acid sequence of VIWRGGSTDYNAAFVS (SEQ ID NO: 2);
(c) an HVR-H3 comprising the amino acid sequence of HYYGSSDYALDN (SEQ ID NO:3);
(d) an HVR-L1 comprising the amino acid sequence of KASQDVRNLVV (SEQ ID NO: 4);
(e) an HVR-L2 comprising the amino acid sequence of SGSYRYS (SEQ ID NO: 5); and
(f) an HVR-L3 comprising the amino acid sequence of QQHYSPPYT (SEQ ID NO: 6), and an anti-CD3 arm comprising a second binding domain comprising the following six HVRs:
(a) an HVR-H1 comprising the amino acid sequence of SYYIH (SEQ ID NO: 9);
(b) an HVR-H2 comprising the amino acid sequence of WIYPENDNTKYNEKFKD (SEQ ID NO: 10);
(c) an HVR-H3 comprising the amino acid sequence of DGYSRYYFDY (SEQ ID NO: 11);
(d) an HVR-L1 comprising the amino acid sequence of KSSQSLLNSRTRKNYLA (SEQ ID NO: 12);
(e) an HVR-L2 comprising the amino acid sequence of WTSTRKS (SEQ ID NO: 13); and
(f) an HVR-L3 comprising the amino acid sequence of KQSFILRT (SEQ ID NO: 14).


SEQUENCE LISTING (38 sequences)

SEQ ID NO: 1 - protein, 5 aa, synthetic construct
RFGVH

SEQ ID NO: 2 - protein, 16 aa, synthetic construct
VIWRGGSTDYNAAFVS

SEQ ID NO: 3 - protein, 12 aa, synthetic construct
HYYGSSDYALDN

SEQ ID NO: 4 - protein, 11 aa, synthetic construct
KASQDVRNLVV

SEQ ID NO: 5 - protein, 7 aa, synthetic construct
SGSYRYS

SEQ ID NO: 6 - protein, 9 aa, synthetic construct
QQHYSPPYT

SEQ ID NO: 7 - protein, 120 aa, synthetic construct
EVQLVESGPGLVKPSETLSLTCTVSGFSLTRFGVHWVRQPPGKGLEWLGVIWRGGSTDYNAAFVSRLTISKDNSKNQVSLKLSSVTAADTAVYYCSNHYYGSSDYALDNWGQGTLVTVSS

SEQ ID NO: 8 - protein, 107 aa, synthetic construct
DIQMTQSPSSLSASVGDRVTITCKASQDVRNLVVWFQQKPGKAPKLLIYSGSYRYSGVPSRFSGSGSGTDFTLTISSLQPEDFATYYCQQHYSPPYTFGQGTKVEIK

SEQ ID NO: 9 - protein, 5 aa, synthetic construct
SYYIH

SEQ ID NO: 10 - protein, 17 aa, synthetic construct
WIYPENDNTKYNEKFKD

SEQ ID NO: 11 - protein, 10 aa, synthetic construct
DGYSRYYFDY

SEQ ID NO: 12 - protein, 17 aa, synthetic construct
KSSQSLLNSRTRKNYLA

SEQ ID NO: 13 - protein, 7 aa, synthetic construct
WTSTRKS

SEQ ID NO: 14 - protein, 8 aa, synthetic construct
KQSFILRT

SEQ ID NO: 15 - protein, 119 aa, synthetic construct
EVQLVQSGAEVKKPGASVKVSCKASGFTFTSYYIHWVRQAPGQGLEWIGWIYPENDNTKYNEKFKDRVTITADTSTSTAYLELSSLRSEDTAVYYCARDGYSRYYFDYWGQGTLVTVSS

SEQ ID NO: 16 - protein, 112 aa, synthetic construct
DIVMTQSPDSLAVSLGERATINCKSSQSLLNSRTRKNYLAWYQQKPGQSPKLLIYWTSTRKSGVPDRFSGSGSGTDFTLTISSLQAEDVAVYYCKQSFILRTFGQGTKVEIK

SEQ ID NO: 17 - protein, 30 aa, synthetic construct
EVQLVESGPGLVKPSETLSLTCTVSGFSLT

SEQ ID NO: 18 - protein, 14 aa, synthetic construct
WVRQPPGKGLEWLG

SEQ ID NO: 19 - protein, 32 aa, synthetic construct
RLTISKDNSKNQVSLKLSSVTAADTAVYYCSN

SEQ ID NO: 20 - protein, 11 aa, synthetic construct
WGQGTLVTVSS

SEQ ID NO: 21 - protein, 23 aa, synthetic construct
DIQMTQSPSSLSASVGDRVTITC

SEQ ID NO: 22 - protein, 15 aa, synthetic construct
WFQQKPGKAPKLLIY

SEQ ID NO: 23 - protein, 32 aa, synthetic construct
GVPSRFSGSGSGTDFTLTISSLQPEDFATYYC

SEQ ID NO: 24 - protein, 10 aa, synthetic construct
FGQGTKVEIK

SEQ ID NO: 25 - protein, 30 aa, synthetic construct
EVQLVQSGAEVKKPGASVKVSCKASGFTFT

SEQ ID NO: 26 - protein, 14 aa, synthetic construct
WVRQAPGQGLEWIG

SEQ ID NO: 27 - protein, 32 aa, synthetic construct
RVTITADTSTSTAYLELSSLRSEDTAVYYCAR

SEQ ID NO: 28 - protein, 11 aa, synthetic construct
WGQGTLVTVSS

SEQ ID NO: 29 - protein, 23 aa, synthetic construct
DIVMTQSPDSLAVSLGERATINC

SEQ ID NO: 30 - protein, 15 aa, synthetic construct
WYQQKPGQSPKLLIY

SEQ ID NO: 31 - protein, 32 aa, synthetic construct
GVPDRFSGSGSGTDFTLTISSLQAEDVAVYYC

SEQ ID NO: 32 - protein, 10 aa, synthetic construct
FGQGTKVEIK

SEQ ID NO: 33 - DNA, 5620 bp, Homo sapiens
  source 1..5620: mol_type = genomic DNA
gcagtttcagaacccagccagcctctctcttgctgcctagcctcctgccggcctcatcttcgcccagccaaccccgcctggagccctatggccaactgcgagttcagcccggtgtccggggacaaaccctgctgccggctctctaggagagcccaactctgtcttggcgtcagtatcctggtcctgatcctcgtcgtggtgctcgcggtggtcgtcccgaggtggcgccagcagtggagcggtccgggcaccaccaagcgctttcccgagaccgtcctggcgcgatgcgtcaagtacactgaaattcatcctgagatgagacatgtagactgccaaagtgtatgggatgctttcaagggtgcatttatttcaaaacatccttgcaacattactgaagaagactatcagccactaatgaagttgggaactcagaccgtaccttgcaacaagattcttctttggagcagaataaaagatctggcccatcagttcacacaggtccagcgggacatgttcaccctggaggacacgctgctaggctaccttgctgatgacctcacatggtgtggtgaattcaacacttccaaaataaactatcaatcttgcccagactggagaaaggactgcagcaacaaccctgtttcagtattctggaaaacggtttcccgcaggtttgcagaagctgcctgtgatgtggtccatgtgatgctcaatggatcccgcagtaaaatctttgacaaaaacagcacttttgggagtgtggaagtccataatttgcaaccagagaaggttcagacactagaggcctgggtgatacatggtggaagagaagattccagagacttatgccaggatcccaccataaaagagctggaatcgattataagcaaaaggaatattcaattttcctgcaagaatatctacagacctgacaagtttcttcagtgtgtgaaaaatcctgaggattcatcttgcacatctgagatctgagccagtcgctgtggttgttttagctccttgactccttgtggtttatgtcatcatacatgactcagcatacctgctggtgcagagctgaagattttggagggtcctccacaataaggtcaatgccagagacggaagcctttttccccaaagtcttaaaataacttatatcatcagcatacctttattgtgatctatcaatagtcaagaaaaattattgtataagattagaatgaaaattgtatgttaagttacttcactttaattctcatgtgatccttttatgttatttatatattggtaacatcctttctattgaaaaatcaccacaccaaacctctcttattagaacaggcaagtgaagaaaagtgaatgctcaagtttttcagaaagcattacatttccaaatgaatgaccttgttgcatgatgtatttttgtacccttcctacagatagtcaaaccataaacttcatggtcatgggtcatgttggtgaaaattattctgtaggatataagctacccacgtacttggtgctttaccccaacccttccaacagtgctgtgaggttggtattatttcattttttagatgagaaaatgggagctcagagaggttatatatttaagttggtgcaaaagtaattgcaagttttgccaccgaaaggaatggcaaaaccacaattatttttgaaccaacctaataatttaccgtaagtcctacatttagtatcaagctagagactgaatttgaactcaactctgtccaactccaaaattcatgtgctttttccttctaggcctttcataccaaactaatagtagtttatattctcttccaacaaatgcatattggattaaattgactagaatggaatctggaatatagttcttctggatggctccaaaacacatgtttttcttcccccgtcttcctcctcctcttcatgctcagtgttttatatatgtagtatacagttaaaatatacttgttgctggtactggcagcttatattttctctcttttttcatggattaaccttgcttgagggctttaacaattgtattactttttcaaagaactaagctttagcttcattgatttttttctatttaattgggttttgctcttctctttagcattggaaacatagaaatgctttctgatttctttgggtagatttacgtattcagcttcttgagatggaagtttagatcactgatccttcagcttgttttcttttttgtatacatagattttaggacgatatattttcccttgagttctgctttagctgcagctcttatgttttgatatgcctctctttattatccttcagttaaaaatatctttcaattcattgttatataaaaatatgtgcctagtttttaacatctggagattttctagttttgaaaaaaacataagccaggcatggtggctcacacctgtatccccagcactttgggaggccgagacgggaggatcgcctgagctcaggagtttttacaccagcctgggaataacagtgagacattatctccaaaaaaattacctgggtatggtgttgtgcacctgtagtcccagctactctggagactgaggtgggaggattgtttgagcttgggaggttgaggctgcagggagctgtgatcacaccactgcactctggcctgagtgacagattgagaccctgtctcaataaaagcaaaaataaagaaaataaaccatatgtgttgaacaaaggattaataaattaatttgagactccttcagggaatgaccacaatttattgaaaatagcctaaatgttggagtcaggcatttctggattcatattttgacatcatgctgtcatcttgaacaaaatgcctaacctttctgaacttcaacttccttgccactcaaataaggattacaaaacttaaaatgtggtaagtactaaagacgacagcaaaaattgagtccagcacagagcttcctaaataagcaagcactcaacagagttggttcctttcttcctcccctgcttgacaatccagtttcccacaggagcctttgtagctgtagccaccatggtcagtccagggattcttcactagccccttctcccctggcagacatccttgtgggagtttagtcttggctcgacatgaggatgggggtttgggaccagttctgagtgagaatcagacttgccccaagttgccattagctccccctgcagaatgtcttcagaatcggggcccggtcagtctcctgggtgacctgctgttttcctcttaagatcctttccactttggttgctgctttcgggactcatcgagtccttgctcaacaggataccccttgaagtggctgcctgggccacatccccttccaaacaagaaatcaaaatattagaaatcaatttttgaaatttcccctaggaagactcatttgagtgttcaagttcagagccagtggagaccttaggggagggtggtcacaaggattttgcacagtgctttagagggtcccagggagccacagaggtggtgaggggctgggtgctcttttctccgtgcatgaccttgtgtgtctatcttcattaccacaatgcctcatctctacctcctttccccctgtagttccaacgtgggtatctttgccatctctggcccgaaggactttctgacctacatgtataaataccccctcacaatatatattacttttcctataagtgacttctctactggattactggttgctcatacacctcatattttactcgtaaatctactactccctgtctgcctactccattctcatttgctgtagaaaattctcttaccatcccaactttcacccaccatcatgcttacccaaaggctgtgggaatgacctgggccctaatgccccttttctaaattcctaaggctcaccattttcctattgtaatggttcttgaccttataatgtttgaggcaccttttcaaatatagtcctttgatttcagactgaatacttgaaaggacacacacacacatacgtaagtgcatatgactgcatacacccacacacacacacgtgcctgtatacagtcatatgatacatacacaaacacacgcacacaagcctgcatacatcatatgccaacagtggggatatgttctgagaaatgcatcattagatgattttgtcattgtgtgaacatcatagagtgtacttacactaacctagatggtctaacctactacacacccaggctacatggtatcacctattcctcctaggctacaagcctgtacagcgtgtgtctgtactaaatgctgtgggcaattttaacctgatggtaaatgtttgtgtatctaaacatatctaaacatagaaaaggtacagtaaacatgcagtattataatcttatgagaccgtcatcatatatgtggtccactgtttgggccatcattggctgaaaagtggttatgcgacacatgactgtatatatactttcctgttacaacaacagtgtctctcaatccacagtaattgcagcatccagtaggtcttactttagccctgagtcaccatttgtgtcaacgtgtttagtgccatgtccacgtctctcatgtaactggcagagctatcaaatattttggcaaaacacattgtttctttggctttgccttggtaactttctgtgccttttgtagctcttgtttggaagaagctcaacccatgtctgcacactgtgatacaagggggacagcatcgacatcgacttacttcttggtgccttattcctccttagaacaattcctaaatctgtaacttaagtttctcaggaagattccatactgcacagaaaactgcttttgtgggtttttaaaaggcaagttgttatatgtgctggatagtttttaagtatgacataaaaattgtataaagtaaaatattaaaatacacctagaatactgtataactttaagtcattttatcaacacattgctaatccagatattttcccgcagtttttctttgaataacagagcaattaatttacttttactatgaagagtcatcattttagtatgtattttaagcaatccaccaagaactcagtaggcagctgagaggtgctgcccagagaagtggtgattagcttggccttagctcacccacacaaagcacaacaggctttgaactattccctaacggggcatttattcttttttttttttttttttgggagacggagtctcgctgtcgcccaggctagagtgcagtggcgcgatctcggctcactgcaggctccaccccctggggttcacgccattctcctgcctcagcctcccaagtagctgggactgcaggcgcccgccatctcgcccggctaattttttgtatttttagtagagacggggtttcaccgtgttagccaggatagggcatttattcttgaacttgattcagagaggcacacattaccattctctaatcagaatgcaagtagcgcaaggcggtggaaactatggaattcggaggcaggtgatgcattgggcgagtttattaacatctgtgactctctagtttgaaatttatttgtaacagacaaaaatgaattaaacaaacaataaaagtataataaagaa

SEQ ID NO: 34 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 35 - protein, 450 aa, synthetic construct
EVQLVESGPGLVKPSETLSLTCTVSGFSLTRFGVHWVRQPPGKGLEWLGVIWRGGSTDYNAAFVSRLTISKDNSKNQVSLKLSSVTAADTAVYYCSNHYYGSSDYALDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 214 aa, synthetic construct
DIQMTQSPSSLSASVGDRVTITCKASQDVRNLVVWFQQKPGKAPKLLIYSGSYRYSGVPSRFSGSGSGTDFTLTISSLQPEDFATYYCQQHYSPPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 449 aa, synthetic construct
EVQLVQSGAEVKKPGASVKVSCKASGFTFTSYYIHWVRQAPGQGLEWIGWIYPENDNTKYNEKFKDRVTITADTSTSTAYLELSSLRSEDTAVYYCARDGYSRYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 38 - protein, 219 aa, synthetic construct
DIVMTQSPDSLAVSLGERATINCKSSQSLLNSRTRKNYLAWYQQKPGQSPKLLIYWTSTRKSGVPDRFSGSGSGTDFTLTISSLQAEDVAVYYCKQSFILRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC